(19) United States
(12) Patent Application Publication
(10) Pub. No.: US 2003/0143554 A1
(43) Pub. Date: Jul. 31, 2003

(54) Method of genotyping by determination of allele copy number

(76) Inventors: Mark E. Berres, Madison, WI (US); John A.W. Kirsch, Madison, WI (US); William R. Engels, Madison, WI (US)

Correspondence Address: David D. Stein, Boyle, Fredrickson, Newholm, Stein & Gratz, S.C., Suite 1030, 250 E. Wisconsin Avenue, Milwaukee, WI, 53202, US

(21) Appl. No.: 10/112,882

(22) Filed: Mar. 29, 2002

Related U.S. Application Data
(60) Provisional application No. 60/280,727, filed on Mar. 31, 2001.
(60) Provisional application No. 60/313,578, filed on Aug. 17, 2001.

(51) Int. Cl. C12Q 1/68; G06F 19/00; G01N 33/48; G01N 33/50

(52) U.S. Cl. 435/6; 702/20

(57) ABSTRACT

The majority of PCR-based fingerprinting technologies generate dominant genetic markers; homozygote present and heterozygote genotypes cannot be distinguished using conventional detection methods. In contrast, codominant genetic markers provide an unambiguous distinction among each genotype. A genotyping method is described that includes procedures implemented in software. This method quantifies allele copy number and enables recovery of codominant genotypes from markers expressing ostensibly dominant phenotypes. These procedures are designed and implemented to (1) greatly reduce variability attributable to sample assay and detector noise, (2) accurately estimate allele size and copy number, (3) provide normalization criteria for intra- and inter-marker comparisons, and (4) scale the resulting data to determine the genotype of individual markers.

### CROSS REFERENCE TO RELATED APPLICATIONS

This application claims priority under 35 U.S.C. §119(e) to U.S. Provisional Patent Application No. 60/280,727, filed Mar. 31, 2001, and U.S. Provisional Patent Application No. 60/313,578, filed Aug. 17, 2001, the entirety of both of which are incorporated by reference herein.

### REFERENCE TO GOVERNMENT GRANT

This invention was made with United States government support awarded by the National Institutes of Health, Grant #NIH GM30948 and the National Science Foundation, Grant #DEB-0073432. The United States has certain rights in this invention.

### FIELD OF THE INVENTION

The invention relates to a method that is used in genotyping as well as to methods of processing data, typically photometric data, which can be used in genotyping and related applications, and more particularly to a method for determining allele copy number using one or more such methods.

### DESCRIPTION OF THE RELATED ART

Genetic analysis requires tools for accurately and precisely defining the genetic composition of individuals. Many different genetic technologies have been developed for this purpose, but no single technique is universally applicable to all types of genetic analyses. For example, DNA sequencing technologies recover the precise nucleotide sequence of a targeted (and often very small) region of the entire genome. Numerous studies have shown that this approach readily detects variation among individuals at or above the generic level. However, as typically practiced, nucleotide sequencing generally does not detect variation below the generic level (e.g. populations of a species) due to increased relatedness among individuals. A new class of genetic markers, collectively known as DNA fingerprinting technologies, overcomes many of the current limitations of polymorphism detection inherent to small-scale nucleotide sequencing.

In its most general sense, DNA fingerprinting, (or DNA profiling), refers to any technology that characterizes an individual's DNA. Most often, this characterization is embodied as some manipulation of the host's chromosomal material followed by a detection procedure, which enables visualization of the test results. Any differences observed between individuals indicate a genetic difference (polymorphism) at a level of resolution dictated by the original characterization procedure, most often individual nucleotides. Generally, the test results of most characterization and detection procedures consist of DNA fragments separated and visualized in an electrophoretic gel. In one best-case scenario, observed DNA fragments will form a pattern unique to each individual sampled. However, depending on the type of genetic characterization employed, not all DNA fingerprints may uniquely identify every individual.

The ability of any genetic technology to discriminate among individuals depends on many important criteria including: (1) the accuracy and precision of the genetic fingerprinting technology employed, (2) the underlying basis of marker polymorphism, (3) the number of markers generated per reaction (multiplex content) and the number of alleles per marker (information content), and especially (4) the ability to discriminate between homozygote and heterozygote genotypes. Despite the large number of DNA fingerprinting technologies currently available, few, if any, exhibit even a majority of the aforementioned criteria of a genetic marker system. This fact is further exacerbated when considering costs of time and financial investment associated with a particular genetic marker technology.

For example, in RAPD (Randomly Amplified Polymorphic DNA) markers, random segments of DNA are generated with PCR amplification using short primers of arbitrary nucleotide sequence. Amplification products may be present or absent between individuals reflecting changes in the presence or absence of presumably homologous priming sites. The short lengths of the primers demand that the PCR amplification occur under conditions of low selectivity (stringency). However, low stringency greatly increases the chance of nonspecific priming (primer mismatches) thus generating artifactual DNA fragments ultimately leading to erroneous estimates of polymorphism. Moreover, a great deal of experimental evidence suggests that nonspecific priming events are extremely dependent on individual PCR reaction conditions making reproducibility very difficult. As with most PCR-based fingerprinting technologies, RAPD markers cannot discriminate between homozygote and heterozygote genotypes (cf. dominant markers). This inability significantly reduces the information content of RAPD markers although in certain cases, detailed pedigrees may allow identification of heterozygous individuals.

Simple Sequence Repeat (SSR) markers are another PCR-based technique where differences in the number of tandemly repeated nucleotide motifs are generated by PCR amplification and detected by gel electrophoresis. Polymorphism in the number of repeats is visualized as length variation across the region containing the repeated nucleotide motifs. Although SSR markers typically exhibit high variation in repeat number thus providing a genetic marker with high information content, SSR markers have a multiplex ratio of one, i.e. only a single SSR marker can be generated per reaction. SSR markers can also discriminate among homozygote and heterozygote genotypes (cf. codominant markers). Despite these advantages, development of SSR markers requires considerable amounts of time. SSR markers developed in one taxon can rarely be used in others. Therefore, SSR markers usually need to be developed de novo for each additional taxa. More importantly, the molecular basis of SSR polymorphism is not completely understood creating under-appreciated difficulties with the analytical and/or statistical treatment of SSR-based data.

Restriction Fragment-Length Polymorphism (RFLP) markers also detect genetic variation among individuals. RFLP is a DNA hybridization-based technique that reveals polymorphisms as differences in fragment lengths after treatment with a restriction enzyme and electrophoretic separation of the resulting fragments. Two sources of polymorphism contribute to RFLPs: (1) presence and/or absence of restriction sites that determine the number of RFLP fragments generated and (2) length variation caused by insertions or deletions between restriction sites. At first, RFLPs may appear as an attractive fingerprinting methodology especially as RFLP can discriminate among homozygote and heterozygote genotypes. However, RFLP analysis is a very time consuming process, requires relatively large quantities of target and probe DNA, and, as traditionally performed, uses hazardous radioactive labeling of probe DNA. These shortcomings, further magnified with a multiplex content of one and generally low information content, make RFLP analysis a poor DNA fingerprinting choice.

Alloenzymes, variants of a protein encoded by different alleles at the same locus, have also been widely used to study genetic variation in populations. Although not technically a DNA fingerprinting methodology, protein polymorphisms result from nucleotide base substitutions and thus provide a crude estimate of polymorphism at the DNA level. Nucleotide substitutions in protein-coding loci often lead to the incorporation of different amino acids into the protein. Because of electrical charge variation among individual amino acids, alloenzymes can be differentiated by their relative migration distance during gel electrophoresis. Although alloenzymes are time and cost efficient for research, many protein-coding loci appear invariant within populations (or even at higher taxonomic levels), and most polymorphic enzymes have only a few variants. Recent evidence suggests that this phenomenon may be, in fact, due to experimental artifacts produced by pH gradients affecting the electrical characteristics of the enzyme. In any case, these problems greatly limit the power of alloenzyme analysis to resolve genetic differences among individuals.

As detailed above, each of these fingerprinting techniques exhibit both strengths and weaknesses. In practice, the choice of a particular technique is often a compromise that depends on the research question pursued and the degree of genetic resolution required, as well as on financial constraints and the technical expertise available.

A relatively new technique that shows promise as being an “ideal” DNA fingerprinting technology is Amplified Fragment-Length Polymorphism (AFLP, Vos et al., 1995, Nucleic Acids Research, 23(21) pp. 4407-4414). Briefly, the AFLP technique involves (1) the restriction digestion of total genomic DNA, (2) ligation of adapters, with known sequence, to the digested fragments, and (3) PCR amplification of a subset of these fragments. Products (amplicons) resulting from the AFLP procedure are typically visualized on a denaturing polyacrylamide gel. The resulting amplicons represent a complex DNA fingerprint derived from cleavage sites distributed throughout the entire genome. These amplicons can be isolated and sequenced, but are more often used for establishing unique DNA fingerprints of individuals in a population, without the need for prior sequence information.

The AFLP procedure exhibits almost all of the desirable qualities in a genetic marker system including: (1) a random distribution of sample sites throughout the genome, (2) a high degree of abundance and polymorphism, (3) a mode of evolution that is primarily selectively neutral, (4) a high multiplex and high information content (due to the large number of loci generated), (4) a well understood basis of marker polymorphism, (5) a capacity for high-throughput amenable to automation, and (6) cost effectiveness and safety.

However, a serious limitation to AFLP is its inability to directly distinguish whether an observed amplicon was originally derived from one or two “doses” of template DNA. For example, in a diploid organism, when DNA is processed with the AFLP procedure and an amplicon is generated by PCR amplification, it may be comprised of DNA segments originally amplified from two identical templates, one present on each homologous chromosome pair. Alternatively, the amplicon may be comprised of DNA segments originally amplified from a single template present on only one homologue of the chromosome pair (on which homologue the template resides on is not distinguished). Thus, any detectable amplicon generated by the AFLP process may have been originally derived from either one or two doses of the (assumed) identical DNA template. A third situation also exists in that when neither template is present, no amplicon is produced and is therefore undetectable (null). The above situations are intended to provide examples for the most commonly observed results of the AFLP process and do not reflect all possible template configurations.

Genetically, the template configurations described above are equivalent to defining the DNA templates as alleles and the position they occupy on a chromosome as a locus. Defined in this manner, the aforementioned configurations simply reflect diploid expectations of the three possible genotypes at a single locus containing two alleles. Therefore, respectively, the three genotypic classes may be identified as having 2, 1 or 0 copies (doses) of the allele (DNA template) distributed at the identical locus (position) on both, one, or neither homologous chromosome pair. The three genotypes of amplicons are herein referred to as “homozygote present” (having been derived from 2 copies of the allele), “heterozygote” (having been derived from 1 copy of the allele), and “homozygote absent” (having neither copy of the allele, [null]).

While it is common for multiple alleles to be segregating at a single genetic locus, the AFLP technique cannot generally be used to distinguish among such alleles. This is a direct result of the AFLP technique relying only on template-length variation to discriminate among amplicons. For example, consider six identically-sized amplicons derived from the identical locus in six different individuals. It is possible that one or more of these six amplicons may be comprised of a nucleotide sequence unique to that amplicon. Genetically, each amplicon with a different nucleotide sequence would be considered a unique allele provided it was derived from the same locus. However, since there is no difference in amplicon length, and AFLP-generated amplicons are not routinely sequenced, all six amplicons would be considered derived from identical alleles. In other words, the definition of allele, as is used herein, refers to the original DNA template from which the PCR-generated amplicon was derived. Additionally, allele identification must be restricted to size-homology rather than identity of nucleotide sequence. It is further recognized that in most cases, the exact chromosomal position (i.e. locus) of a given DNA template that results in the production of an amplicon is unknown.

Therefore, all AFLP-generated amplicons present at a given locus, provided they have identical lengths (as indicated by their molecular weight or molecular mobility) are assumed to be identical-in-state (i.e. allelic). Furthermore, it is assumed that the absence of an amplicon, where others are known to exist, indicates a null allele. As a result, AFLP, as traditionally practiced, can only provide an indication of whether or not a particular amplicon is present or absent (cf. phenotype). Thus, even if the amplicon is present, AFLP, as traditionally practiced, still cannot provide any indication of genotype. For most genetic analyses, the ability to distinguish among each genotype is clearly superior to situations where this is not possible: it allows direct estimation of allele frequencies and for any given level of statistical power, requires smaller sample sizes.

A recent improvement in the AFLP technique has been the implementation of fluorescent labels and automated DNA sequencers or analyzers to aid in the visualization of AFLP-generated fingerprints. Fluorescent AFLP (fAFLP) is similar to AFLP but implements a modification to the original AFLP procedure where selective amplification is performed with a fluorophore-labeled primer. fAFLP methodologies have greatly increased the throughput for processing samples. However, the same inability to discriminate among homozygote and heterozygote genotypes in AFLP also exists with fAFLP. That is, current techniques available to analyze fAFLP data generated with fluorophore-labeled primers and visualized on automated DNA sequencers still cannot readily determine whether an amplicon was originally derived from one or two copies of an allele. Thus, despite this enhancement to the original AFLP technique, fAFLP still only provides a phenotypic indication of the presense or absence of any amplicon and still cannot provide any indication of genotype.

While fAFLP remains a powerful genetic technology, it would benefit greatly from a method and system of discriminating between homozygote and heterozygote genotypes, thereby realizing its full potential as a genetic marker system. This capability would dramatically increase the power of the AFLP technology with broad applications in pathotyping, population genetics, quantitative trade loci (QTL) mapping, forensic DNA analysis and many other uses. Such a method is described herein.

### SUMMARY OF THE INVENTION

The present invention is directed toward methods of processing data used in genotyping. A preferred implementation of a method of the invention utilizes one or more procedures that enable genotyping to be performed using an emission-based marker system.

One or more procedures of the method can be implemented to practice some or all of a method of genotyping and the procedures can be used in other biotechnology related data processing applications. Some procedures may not be necessary depending on various factors that include, for example, the type of marker or label system, the type of detection system, the need, convenience, or desire to display specific types of data, as well as other factors. Indeed, it is contemplated that one or more of the procedures of the method can be used or adapted for use in other genotyping methods.

In a preferred implementation, photometric data generated an apparatus that detects emissions from fluorophore-labeled DNA fragments or other types of labels attached thereto is processed. Preprocessing of recorded data can be performed, such as where photometric data must be reformatted or separated and extracted from other data.

The photometric data can be baselined to shift all spectral data to a common baseline of zero spectral amplitude. In one preferred baselining procedure, the data is baselined using a regression technique that preferably is multipoint (piecewise) linear regression. For example, the data can be arranged into subsets of datapoints. The datapoint exhibiting the smallest value in each subset is located and the slope between minimum values in adjacent subsets is computed. A baseline offset correction is interpolated for each datapoint using the computed slope. The data is then baselined by subtracting this value from each respective datapoint.

Where desired, spectral overlap among different fluorophores can also be removed. For example, in a multicomponent system (a system containing more than one detectable fluorophore), the components of fluorescence intensity contributed by fluorophore emissions at wavelengths outside of its principal wavelength or range of wavelengths are removed using a n-fluorophore multicomponenting procedure.

Noise reduction of the photometric data preferably is performed. In a preferred noise reduction procedure, noise is attenuated and artifacts, if present, removed, reduced or compensated. Noise reduction that preferably is spectral-based noise reduction is performed to reduce or eliminate noise spikes that can be of a random nature. Noise reduction can also be performed to reduce low amplitude background noise, such as what remains after baselining adjustments are performed

One preferred procedure for reducing spectral-based noise transforms the photometric data from the time domain into a frequency domain using a Fourier Transform that preferably is a Discrete Hartley Transform. To remove high-amplitude spectral noise (e.g. spectral spikes), one or more high-frequency components of the transformed photometric data are truncated at a truncation point that can be empirically determined. To remove low-amplitude spectral noise (e.g. spectral background), partial truncation of the amplitude of one or more low-frequency components can be performed.

After truncation is completed, the photometric data is transformed back into the time domain using an inverse Fourier Transform that preferably is an inverse Discrete Hartley Transform.

To compensate for, or attenuate artifacts that can be created by manipulating the frequency components, an apodization function can be applied to the frequency components after truncation but before transformation back into the time domain. In a preferred implementation, a Gaussian apodization function is multiplied to the frequency components of the photometric data that remain after truncation.

Where it is desired to display the photometric data, the data can be rescaled into a different format that preferably is a more standardized format than produced by commercially available detection systems. It may also be desirable to rescale the data for other purposes. In one preferred implementation, the photometric data is rescaled into an 8-bit hexadecimal data format that can be read by commercial graphic and image-processing programs. If needed, photometric data can be truncated or compressed in order to facilitate resealing and/or storage.

Where it is desired to display the photometric data where a number of different colors or shades of gray are needed, the data can be mapped accordingly, such as into a false color image. For example, where four different fluorophores are used, the intensities of the four fluorophores are mapped into a three-color space.

Where lane tracking is needed to define individual samples (e.g. in slab gel electrophoresis), it can be performed. Using graphically displayed photometric data, a tracking spline can be constructed that identifies each sample lane. Such a procedure can be manually or algorithmically performed in software. Where the photometric data is generated from a capillary electrophoresis procedure or the like, lane tracking may not be needed. Where present, lane-tracking information can be extracted from the original set of photometric data, such as from raw data derived from the detector. There may be no need to rescale or display data where lane tracking is not performed manually.

In a procedure designed to reduce error in determining the relative position of an amplicon within a sample, a function that preferably is an idealized response function, such as a Gaussian function, is fitted to each amplicon in the photometric data. The maximum spectral intensity (peak) of each amplicon is located and fitted with an idealized mathematical function that, if desired, preferably replaces the original data. In locating the spectral peak of an amplicon, a leading edge and/or trailing edge of the amplicon is detected and thereafter the peak apex is located. The apex position is designated when a location, typically a location designated by a scan number, is found where the spectral amplitude of a single amplicon is at a maximum intensity.

Three parameters are used in fitting the peak with an idealized peak-response function. Such parameters preferably include the position of the peak apex, the amplitude at the peak apex, and the variance (e.g., width) of the amplicon. Preferably, amplicon variance is defined as the width of the amplicon where the amplitude of the peak apex is one-half its maximum amplitude.

In a preferred implementation of a procedure for fitting the peak with an idealized peak function, estimates of the three peak parameters are inputted into a regression procedure. A preferred regression procedure uses orthogonal distance regression to produce an idealized peak function based on a Gaussian function. Upon convergence, the procedure produces more accurate estimates of peak apex location, peak apex amplitude, and amplicon variance. If desired, the fitted Gaussian function then replaces the data to which it was fit. Preferably, this procedure is repeated for each amplicon located in the photometric data and can be implemented in a manner that fits the Gaussian function simultaneously to all of the amplicons. In order to estimate the relative size of size-unknown sample amplicons, a size-standard is preferably electrophoresed concurrently with the size-unknown amplicons, preferably within each sample lane.

After fitting both sample amplicons and DNA size standards with the Gaussian function, a sizing curve or sizing function is generated using the fitted parameters for each DNA standard in the set of DNA size standards. The sizing curve or function is thereafter used to estimate the size (e.g. in units of molecular weight or molecular mobility) of each sample amplicon that is of unknown size.

In a preferred procedure for generating such a sizing curve or sizing function, a locally quadratic, weighted regression approach is used. In a preferred implementation, the estimated peak parameters for a number of standardized DNA fragments less than the total number of standards (a “neighborhood”) is used in determining sizing functions for each individual standard fragment. The number of DNA fragments used in determining a sizing function for a particular DNA standard is selected so as to minimize the residual error of a least-squares minimization fit to differently-sized subsets of standard fragments. Once the sizing curve is generated, the sizes of size-unknown sample amplicons may be predicted by using the estimated peak apex position of each size-unknown sample amplicon.

To transform the continuously-valued sizes into discrete representations of identically-sized amplicons, a binning operation preferably is performed using the estimated sizes of all sample amplicons. Any two amplicons are considered homologous (identical-in-state) if the absolute difference in their estimated size is less than or equal to the largest residual of size in all of the size estimates for a given sizing curve. In a preferred implementation, a matrix is created that has an element corresponding to each amplicon and holds a value (e.g., 1 or 0) depending upon whether, at a particular amplicon size-class (cf. locus), an amplicon is either present (1) or absent (0) in each sample analyzed. Each such locus holding uniform values of 1 across all samples is designated as being putatively monomorphic. Any locus holding at least one value of 0 is designated as being polymorphic.

Using the parameters obtained from the photometric data that correspond to each amplicon, the spectral intensity of the amplicon is estimated using a model that describes the expectations of ssDNA diffusion and displacement in a denaturing electrophoretic gel matrix. The spectral intensities of all amplicons at each putatively designated monomorphic locus are estimated to determine a locus that can be designated as a reference monomorphic locus. This reference locus is used to estimate normalizing coefficients that, when applied to the spectral intensities of other amplicons, will enable direct comparison of relative spectral intensities of amplicons at other loci. Once normalized, the spectral intensities of all other amplicons are able to be genotyped.

In a preferred implementation of a procedure for estimating the spectral intensity of an amplicon, the estimated parameters of peak apex amplitude and amplicon variance are used. One preferred implementation of a two-dimensional procedure produces an analytical result equal to the square root of four multiplied by π multiplied by the estimated peak variance associated with the amplicon multiplied by its peak apex amplitude. The estimated spectral intensity preferably is computed for each amplicon in the sample.

In another preferred implementation, the spectral intensity of each amplicon is estimated by fitting a thin-plate spline to the spectral data and then determining the area under the surface by numerical integration.

In selecting a reference monomorphic locus, the variability in spectral intensity of each amplicon of each putatively monomorphic locus is determined. The locus having the least amount of variability in spectral intensity, as determined by a coefficient of variation, amongst all amplicons at that locus, is designated as a reference monomorphic locus. It is assumed that the reference monomorphic locus is truly monomorphic.

In a preferred procedure for calculating normalizing coefficients for estimated spectral intensities, the spectral intensities of the amplicons at the reference monomorphic locus are used to determine a normalizing coefficient for all amplicons in each individual sample. The computed normalizing coefficients of each sample are applied to the estimated spectral intensity of each amplicon within that sample.

In one preferred implementation, the average spectral intensity of all of the amplicons at the reference monomorphic locus is determined. The average spectral intensity is then used to compute the normalizing coefficient for each sample. Preferably, the estimated spectral intensity of each amplicon at the reference monomorphic locus is divided by the average spectral intensity to obtain a normalizing coefficient for each individual sample. Thereafter, all of the spectral intensities of amplicons at other loci are normalized by multiplying the spectral intensity of each amplicon by the normalizing coefficient associated with that sample.

In a preferred implementation of a genotyping procedure, the normalized values of spectral intensity of each amplicon are scaled to unity. Thereafter, the scale values are used to assign a genotype to each amplicon at each locus. Preferably, this procedure clearly separates the scaled values of normalized spectral intensity such that they can be placed into three genotypic classes by visual inspection: homozygote present, heterozygote, and homozygote absent (null). Preferably, a genotype of homozygote absent is assigned to each amplicon exhibiting a normalized spectral intensity of approximately zero. Preferably, a genotype of homozygote present is assigned to each amplicon exhibiting a normalized spectral intensity of about one. A heterozygote genotype is assigned to each amplicon exhibiting an intermediate normalized spectral intensity that preferably is about one-half of the normalized spectral intensity of amplicons having a homozygote present genotype.

In one preferred implementation of a method of genotyping, each procedure is used with one or more of the other procedures implemented in software or firmware using a computer equipped with a processor, memory and other computer components, such as a keyboard, a mouse, a display, etc., as needed. Such a computer can be a stand-alone device or can be integrated with some other apparatus, such as a detector or some other apparatus. Where lane tracking information is not needed or can be obtained in another manner, it may not be necessary to rescale and/or graphically display the data. If desired, one or more of these procedures can be used in conjunction with other genotyping methods.

In a preferred implementation of a method of genotyping, one or more of these procedures are used to process photometric data obtained from fragments of DNA prepared with fAFLP. While such photometric data will contain spectral intensity information from detection of detectable wavelengths emitted by markers attached to the fragments, the method and procedures of the method are also well suited for use where the photometric data contains information or data obtained using other detection processes. For example, the method and procedures of the method can be used to process photometric data obtained from detection of light that need not be visible to the naked eye.

It is an object of the invention to provide an improved genotyping method that can be used to determine allele copy number.

It is another object of the inveniton to provide an improved genotyping method that can be implemented in software.

It is still another object of the invention to provide stand-alone procedures that can be implemented in software that can be used alone or in combination with one or more procedures that make up the above-described genotyping method or another genotyping method.

It is a further object of the invention to provide an improved genotyping method compared to traditionally-practiced AFLP and other methods of polymorphism screening and detection which do not directly allow discrimination between homozygote and heterozygote genotypes.

It is a still further object of the invention to provide a method of genotyping to that includes one or more procedures that are implemented in software. Depending on the specific polymorphism screening and detection procedures, one or more of the implemented procedures may not be necessary to fully realize the improved genotyping process.

It is an object of the invention to provide a method of genotyping that increases the certainty of genotyping when compared to other methods of polymorphism screening and detection, which do not directly allow discrimination between homozygote and heterozygote genotypes.

It is an object of the invention to provide a method of genotyping that baselines photometric data, removes spectral overlap, attenuates spectral noise, identifies spectral peaks in the photometric data as amplicons to be genotyped, estimates a plurality of peak parameters for each amplicon, provides amplicon sizing information, bins amplicons, assigns polymorphic and putatively monomorphic loci, estimates the spectral intensity of the amplicons, assigns a reference monomorphic locus, normalizes amplicon spectral intensity, and assigns genotypes to amplicons based on the relative intensity of the normalized spectral intensities.

It is another object of the invention to provide a procedure that can be used to more accurately estimate the size (e.g. in units molecular weight or molecular mobility) of a DNA fragment that can be a PCR-generated amplicon.

It is still another object of the invention to reduce marker assay error, loading volume error, electrophoresis error, and/or detector error using one or more of the aforementioned procedures.

It is a further object of the invention to estimate the spectral intensity of an amplicon to infer allele dosage (2, 1, or 0 doses), which in turn is a direct indicator of genotype (homozygote present, heterozygote, or homozygote absent [null]).

It is an advantage of the invention that processing a plurality of amplicons in a plurality of samples provides more accurate genotyping results.

It is another advantage of the invention that allele copy number determination using the method as well as one or more select procedures that make up the method or another like method can be performed in an automated manner using a computer equipped with a processor and memory.

It is another advantage of the invention that genotyping using the method as well as select procedures of the method can be performed in an automated manner using a computer equipped with a processor and memory.

It is still another advantage of the invention that allele copy number determination using the method as well as one or more procedures that make up the method can be performed in an automated manner using a processor that interfaces with a memory.

It is a still further advantage of the invention that one or more of the procedures used in the genotyping method can be used separately and independently.

It is still another advantage of the invention that estimation of the size (e.g., molecular weight or molecular mobility) of each amplicon is more precise.

It is still another advantage of the invention that genotyping error is reduced.

It is still another advantage of the invention in that it does not require prior knowledge of pedigree information to assign genotypes.

It is still another advantage of the invention in that it enables the recovery of codominant genotypes from dominant phenotypic data.

### BRIEF DESCRIPTION OF THE DRAWINGS

These and other objects, features and advantages of this invention will be apparent in view of the following detailed description of the best mode, appended claims, and accompanying drawings in which:

FIG. 1 is flowchart outlining the steps of a preferred fluorescent-based genotyping method in accordance with the invention;

FIG. 2 is a diagram depicting a generalized fAFLP technique;

FIG. 3 is a diagram depicting plateau-phase effects on DNA concentration in PCR-amplified amplicons;

FIG. 4 is a graph showing raw fluorophore emissions of a relatively large set of scans derived from one-dimensional source data;

FIG. 5 is an enlarged portion of a graph depicting raw fluorophore emissions prior to baseline adjustment;

FIG. 6 is a graph depicting the fluorophore emissions shown in FIG. 5 after baseline adjustment;

FIG. 7 is a graph showing fluorophore emissions for a relatively large set of scans after application of a baseline adjustment to the data shown in FIG. 4;

FIG. 8 is a graph depicting emission spectra of four different fluorophores illustrating the phenomenon of spectral overlap;

FIG. 9 is a graph depicting fluorophore emissions after performing baseline adjustment and after performing a four-fluorophore multicomponent procedure;

FIG. 10 is a graph depicting baselined and multicomponented fluorophore emissions of five molecular-weight standards prior to noise reduction;

FIG. 11 depicts a frequency-domain graph of a Discrete Hartley Transform (DHT) of the time-domain spectral emission data illustrated in FIG. 10;

FIG. 12 is a graph illustrating a Gaussian apodization function;

FIG. 13 is a graph depicting truncated and Gaussian apodized data derived from the DHT illustrated in FIG. 11;

FIG. 14 is a graph illustrating baselined and multicomponented fluorophore emissions of the five molecular-weight standards illustrated in FIG. 10 after noise reduction;

FIG. 15 is a graph showing an example of baselined and multicomponented fluorophore emissions of a single noise-spike prior to noise reduction;

FIG. 16 is a graph depicting baselined and multicomponented fluorophore emissions of the noise spike after noise reduction;

FIG. 17A illustrates an example of raw spectral data taken from two-dimensional source data;

FIG. 17B illustrates the same data after performing baselining, multicomponenting and noise reduction procedures;

FIG. 18A is a graph illustrating a single baselined, multicomponented and noise reduced spectral peak derived from one-dimensional source data;

FIG. 18B is a graph illustrating a portion of the leading edge of the peak shown in FIG. 18A used in determining peak variance;

FIG. 18C illustrates the peak shown in FIG. 18A along with a Gaussian peak, shown in phantom, fitted thereto using orthogonal distance regression;

FIG. 19A is a graph depicting five baselined, multicomponented, and noise-reduced spectral peaks derived from one dimensional source data;

FIG. 19B is a graph displaying the peaks shown in FIG. 19A each fitted with a Gaussian peak shown in phantom;

FIG. 20 is an exemplary sizing curve generated from 22 DNA standards;

FIG. 21A is a contour plot illustrating the distribution of spectral intensities within a single amplicon derived from two-dimensional data;

FIG. 21B is a contour plot showing anomalous distribution of spectral intensities due to amplicon passage through an anisotropic portion of a gel matrix;

FIG. 22A illustrates a three-dimensional plot of baselined, multicomponented, and noise-reduced spectral intensity data of the amplicon shown in FIG. 21A;

FIG. 22B illustrates the plot of FIG. 22A after fitting a Gaussian function to it using orthogonal distance regression;

FIG. 23A is a contour plot of processed spectral data for a single amplicon;

FIG. 23B is a surface plot of a fitted TPS function to the data in FIG. 23A;

FIG. 23C is a contour plot of a fitted Gaussian function to the data in FIG. 23A;

FIG. 23D is a contour plot of the fitted TPS function to the data in FIG. 23A;

FIG. 24 is a diagram depicting genotypes located for nine autosomal (1-9) and two sex-linked loci (10 and 11) in Domestic Chicken (Gallus gallus) using a preferred embodiment of a method of the invention.

Before explaining embodiments of the invention in detail, it is to be understood that the invention is not limited in its application to the details of construction and the arrangement of the components set forth in the following description or illustrated in the drawings. The invention is capable of other embodiments or being practiced or carried out in various ways. Also, it is to be understood that the phraseology and terminology employed herein is for the purpose of description and should not be regarded as limiting.

### DETAILED DESCRIPTION OF AT LEAST ONE PREFERRED EMBODIMENT OF THE INVENTION

### Terminology and Definitions

The following definitions are intended to assist in providing a clear and consistent understanding of the scope and detail of the terms:

AFLP. Amplified Fragment-Length Polymorphism. A technology used to produce a specific type of DNA fingerprint. In its original form, the AFLP process used radioisotopes to visualize AFLP-generated DNA fragments. See, e.g., U.S. Pat. No. 6,045,994, which is expressly incorporated herein by reference, for a description of the process.

Allele. A fAFLP/AFLP-generated DNA template derived from corresponding loci on either homologous chromosome and categorized by size (molecular weight or molecular mobility). In one preferred embodiment of the method described herein, the chromosome from which the fAFLP/AFLP allele originates is not distinguished. See locus.

Amplicon. A quantity of PCR-amplified, identically-sized DNA molecules (fragments) derived from a specific DNA template (allele). The term amplicon is used herein to generally describe any DNA fragment or band produced by the AFLP/fAFLP process and suitably detected.

Aneuploid. An organism (or cell) in which the chromosome number is not an exact multiple of the haploid number. See genotype.

Channel. With particular reference to ABI-based detectors, a channel is a one-dimensional vector of data that refers to the number of read regions that are scanned across a gel or capillary (e.g. from left to right) during electrophoresis. In Applied Biosystems (ABI) slab-gel machines, the number of channels varies with options specific to each machine. For example, the number of channels in a 377, 377XL and 377-96 are 194, 388, and 480, respectively. For ABI capillary-based machines, e.g. ABI 3100, there are three channels. Similarly, with other capillary-based machines, the number of channels can also vary. See scan.

Chromatogram. A generalized reference to a computer file containing photometric data. For example, see gelfile or sample file.

Codominant marker. Any marker that allows discrimination of homozygote and heterozygote genotypes. See genotype.

DNA. Deoxyribonucleic acid. An organic polymer composed of four nitrogenous bases (guanine, adenine, thymine, and cytosine) linked via intervening units of phosphate and the pentose sugar deoxyribose.

DNA fingerprint. Any method that generates profiles of DNA based on the manipulation of the host's chromosomal material.

DNA fragment. A small segment of DNA, restricted here to those DNA fragments generated by the fAFLP/AFLP process. See allele.

Dominant marker. Any marker that is scored as either present or absent (null). Dominant markers cannot distinguish between homozygote and heterozygote genotypes. See genotype.

Electropherogram. A generalized reference to the physical display, in two dimensions, of photometric data, typically by scan number (X dimension) and spectral amplitude (Y dimension). For example, see sample file.

FAFLP. Fluorescent Amplified Fragment-Length Polymorphism. This procedure is identical to AFLP except that it uses fluorescent labels (e.g., fluorophores) and instruments, such as automated DNA sequencers or densitometers, to detect fAFLP-generated DNA fragments.

Filter. With particular reference to ABI-based CCD detectors, fluorophore emissions are collected as signal intensities recorded from specific locations on a charged-coupled device (CCD) instrument. These locations are sensitive only to particular wavelengths of light. Thus, the CCD instrument filters predetermined wavelengths of light similar to how physical filters separate individual wavelengths. However, since no physical filtering is actually performed, CCD filtering in this manner is referred to as a virtual filter. The majority of ABI-based CCD instruments possess four virtual filters each corresponding to a specific range of detectable wavelengths.

Fluorophore. A fluorescent moiety that can be attached covalently to a DNA fragment (typically the 5′ position). Commonly used fluorophores include 5-FAM (5-carboxyfluorescein), 6-FAM (6-carboxyfluorescein), HEX (6-carboxy-1,4-dichloro-2′,4′,5′,7′-tetra-chlorofluorescein), JOE (6-carboxy-4′,5′-dichloro-2′,7′-dimethoxy-fluorescein), NED (proprietary), TAMRA (6-carboxytetramethylrhodamine), TET (6-carboxy-1,4-dichloro-2′,7′-dichloro-fluorescein), and ROX (6-carboxy-X-rhodamine).

Fragment binning. A method by which molecular weights or mobilities of fAFLP/AFLP generated amplicons are converted from continuous values (representing size) to discrete values indicating the presence or absence of a given amplicon of a specific size in a specific individual. See sizing curve.

Gel electrophoresis. The movement of charged molecules through a gel or polymer matrix in an electrical field. It is used herein to define the separation of DNA fragments based on their size (molecular weight or molecular mobility).

Gelfile. A computer file containing the raw fluorescence data (and possibly other information) recorded from all samples processed on a slab-based automated sequencer. Gelfiles are operationally two-dimensional i.e. one dimension contains the individual samples (e.g. X) which are electrophoresed in the second dimension (e.g. Y). For example, one type of gelfile is an ABI gelfile produced using an ABI 377XL automated DNA sequencer. This gelfile is generated with proprietary ABI Collection Software and organized in an unpublished format. See sample file.

Genotype. Agenetically distinct class of amplicon inferred from the number of DNA templates (alleles) present at a specific locus from which the amplicon was originally derived. In a model described herein, it is assumed that, but not limited to, at any single locus, there may be at most, two alleles that could be detected and amplified by the AFLP/fAFLP and PCR process, respectively. Operationally, it is assumed (but not required) that alleles from the identical locus are identical-in-state (i.e. contain the identical nucleotide sequence) if they have the same size as determined by, for example, their molecular weight or mobility in an electrophoretic gel. Therefore, for example in diploid systems, only three possible genotypes exist: a homozygote for the presence of the allele (two copies [doses] of the DNA template are present, one on each homologous chromosome), heterozygote for the presence of the allele (one copy [dose] of the DNA template is present [on which homologue the template resides on is not distinguished]) or a homozygote for the absence of the allele (neither copy [dose] of the DNA template is present on either homologue [null]). Although the model described herein makes reference only to genotypic configurations present in normal diploid organisms, the model can easily accommodate genotypes in aneuploid or polyploid individuals. In accordance with the invention described herein, the genotype of a locus is thus inferred by the relative amounts of PCR-amplified template DNA present in individual amplicons. That is, the intensity of an amplicon is used to infer allele dosage (2, 1, or 0 doses) which in turn is a direct indicator of genotype (homozygote present, heterozygote, or homozygote absent [null]).

With regard to classical genetic terminology, the results of PCR-based assays are actually amplification products (amplicons), observed in a gel and represent a phenotype as the genotype is typically indistinguishable (cf. dominant marker). This distinction is important because confusion with classical genetic terminology incurs definitional ambiguities. For example, amplicons do not have to be derived from a gene per se (and therefore need not be an allele sensu stricto). References to alleles contained herein are made without regard to functionality but rather to homologous chromosomal position (i.e. a locus).

To understand why a PCR-derived amplicon in a gel is a phenotype, rather than a gene or a genotype, first consider DNA templates that do or do not support PCR amplification. Those templates capable of supporting amplification are defined as [1] and those not capable of supporting amplification are defined as [0]. Therefore, in diploid systems, three possible genotypic states exist: [1:1], [1:0], and [0:0]. Homozygous [1:1] and heterozygous [1:0] templates both support amplification whereas the [0:0] template does not. Thus, the presence of an amplicon signifies a phenotype associated with both [1:1] and [1:0] templates; the absence of an amplicon represents the phenotype of [0:0]. Therefore, the [1] template is dominant to the recessive [0] template because it is present in both homozygous [1:1] and heterozygous [1:0] phenotypes. These precise definitions provide a basis for using classical genetic terminology applied synonymously to amplicon patterns observed in electrophoretic gels:

| | Classical allelic | | Operational amplicon |
|---|---|---|---|
| | definition. | | definition. |
| Phenotype | [1] | Dominant | Present |
| | [0] | Recessive | Absent (null) |
| Genotype | [1:1] | Homozygous dominant | Homozygous present |
| | [1:0] | Heterozygous | Heterozygous |
| | [0:0] | Homozygous recessive | Homozygous absent (null) |

Heterozygous. Having only one template (allele) at the corresponding loci of either homologous chromosome. See genotype.

Homozygous absent. Having neither template (allele) at the corresponding loci of both homologous chromosomes. See genotype.

Homozygous present. Having both templates (alleles) at the corresponding loci of both homologous chromosomes. See genotype.

Information content. The number of alleles per marker in a sample of individuals.

Ligation. The process of joining two pieces of DNA (or RNA) with ATP-dependent enzymes called DNA (or RNA) ligases.

Locus. The classification of a fAFLP/AFLP-generated fragment derived from a specific combination of restriction enzymes and PCR amplification primers, defined by its size (molecular weight or molecular mobility). See allele.

Marker. See locus.

Molecular mobility. A physical characterization of molecular size, restricted here to include measurements of DNA fragments with units of length defined by nucleotide bases.

Molecular weight. A physical characterization of molecular size, restricted here to include measurements of DNA fragments with units of length defined by nucleotide bases.

Monomorphic. The existence of a single, genetically distinct class of genotype at a specific locus. See genotype.

Multicomponenting. The process of removing fluorescence overlap between different fluorophores in a multicomponent detection system.

Multiplex ratio. The number of markers detectable in a single reaction.

Nucleic acid. A family of molecules that includes RNA and DNA molecules. More specifically, nucleic acid is the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogues thereof, such as phosphorothioates and thioesters, in either single stranded form, or a double-stranded helix. Double stranded DNA-DNA, DNA-RNA and RNA-RNA helices are possible.

PCR. Polymerase chain reaction (PCR) is an enzyme-catalyzed, in vitro copying process of specific DNA sequences that can use extremely small amounts of template DNA. The process includes cycles of denaturation, annealing with primer, and extension with DNA polymerase are used to amplify the number of copies of a target DNA sequence by >10⁶ times. The PCR allows the selective amplification of specific DNA sequences. The PCR process for amplifying nucleic acids is disclosed in U.S. Pat. Nos. 4,683,195, and 4,683,202, which are incorporated herein by reference for a description of the process.

Phenotype. The observed manifestation of a genotype. The phenotype may be expressed physically, biochemically, or physiologically. See genotype.

Photometric data. Refers specifically to data comprised related to light that need not be visible. Photometric data typically relates to fluorophore emission intensities where fluorophores are used. In the case of ABI-generated gelfiles, the photometric data is a matrix of spectral intensities from the number of channels across the gel (e.g. 194, 388, or 480) from four filters (0, 1, 2, and 4), multiplied by the number of scans (variable) collected. In the case of ABI-generated sample files, the photometric is organized as described above, but with only a single channel of data.

Plateau phase. Refers to the phenomenon of non-exponential, and typically linear, production of amplicons during the later cycle numbers of the PCR amplification process.

Polymerase chain reaction. See PCR.

Polymorphic. The existence of two or more genetically distinct classes of genotype at a specific locus. See genotype.

Polyploid. An organism, such as a cell, in which the chromosome number is an exact integer multiple of two or more complete sets of chromosomes. See genotype.

Primer. A relatively short single-stranded sequence that can bind or anneal to a complementary sequence and can serve as a starting point for DNA synthesis in a PCR reaction.

Putatively monomorphic. With reference to amplicon binning, the existence of a single phenotypic class (i.e. the presence of an amplicon) at a locus.

Quantitative trait loci (QTL). Regions of the genome affecting variation in quantitative (phenotypic) traits. QTLs are identified by (1) generating genotypic markers in individuals with a known pedigree, (2) creating a linkage map that shows the order of the markers and relative distance (in centimorgans, cM) between them, and (3) testing for statistical associations between markers (genotype) and phenotypic expression of the trait(s) of interest.

Restriction enzyme. Enzymes that recognize and cut (restrict) double-stranded DNA at specific short nucleotide sequences. The most commonly used restriction enzymes recognize sequences of four to eight nucleotides in length. Restriction enzymes used in the fAFLP/AFLP protocol produce ends with an overhang of one or more nucleotide bases.

Restriction site. The nucleotide base-pair sequence of a DNA molecule recognized by a specific restriction endonuclease.

RNA. Ribonucleic acid. An organic polymer composed of four nitrogenous bases (guanine, adenine, uridine, and cytosine) linked via intervening units of phosphate and the pentose sugar ribose.

Sample file. A computer file containing processed (and raw) photometric data associated with a single sample. Operationally, sample files are one-dimensional as they either (1) represent a subset of processed photometric data, often derived from slab-based gelfiles, or (2) represent samples derived from individual capillaries in capillary gel electrophoresis. For example, an ABI gelfile is processed with ABI proprietary software (e.g. Genescan Analysis Software) to produce a sample file. The sample file is organized in an unpublished format designed by ABI.

Scan. With particular reference to ABI-based detectors, a scan refers to the number of traverses across the gel made by the scanning laser. For an ABI-based detector, one complete scan corresponds to four traverses of the laser or exciting device. See traverse.

Sizing curve. A method to predict the size (e.g. in units of molecular weight or mobility) of size-unknown amplicons that is based on a mathematical relationship between fragment mobility and molecular size of a known DNA standard.

Spectral overlap. See multicomponenting.

Tracefile. See sample file.

Traverse. With particular reference to ABI-based detectors, one traverse is defined as the path the scanning laser makes from channel 0 to the largest channel supported by the specific machine. See scan.

### Overview

Traditionally, analysis of fAFLP/AFLP data has been limited to scoring only for the presence or absence, i.e., phenotype, of a particular amplicon because no methodology currently exists to reliably distinguish among confounded homozygote present and heterozygote genotypes in such analyses. In other words, as traditionally-practiced, fAFLP/AFLP cannot produce an intrinsically quantitative result that can distinguish whether an observed amplicon was originally derived from one or two copies of template DNA (alleles). This characteristic results in substantial analytical drawbacks because it reduces total information content and poses peculiar statistical biases not always readily tractable. A method is described herein that can distinguish whether an AFLP-processed and PCR-generated amplicon was originally derived from one or two alleles of template DNA, thereby allowing discrimination among homozygote present and heterozygote genotypes. This method is advantageous as it renders the fAFLP/AFLP technology to serve as a codominant marker system.

A preferred implementation of a method of this invention is based on the precise quantitation of spectral emissions generated by induced fluorescence of fluorophore-labeled DNA molecules generated in the FAFLP process. Preferably, the spectral intensity of an amplicon is used to infer allelic dosage (2, 1, or 0 doses), which in turn provides a direct indicator of genotype (homozygote present, heterozygote, or homozygote absent [null]). A preferred implementation of the method assumes that the spectral intensity of PCR-amplified DNA fragments comprising an amplicon is directly proportional to the actual number of DNA molecules contained therein. Since only fluorophore-labeled DNA molecules are detected, the total spectral intensity of all fluorophore-labeled DNA molecules in an amplicon is therefore a good estimate of the actual number of DNA molecules contained within the amplicon. Assuming at most, but not limited to, two identically sized DNA templates (alleles) per locus, heterozygous amplicons are expected to exhibit approximately one-half the spectral intensity as a homozygous amplicon having been originally derived from two copies (doses) of the DNA template. The alternative homozygote produces no spectral intensity as neither copy of the DNA template is present on either homologous chromosome and therefore is not amplified. Thus, the difference in spectral intensity between genotypes is attributable directly to relative differences in the amount of starting template and the number of PCR cycles performed. Overall, the physical amount of DNA present in a fAFLP/AFLP-generated marker, as inferred by the amount of spectral intensity exhibited by the marker, can indicate genotype: heterozygote genotypes should exhibit an intermediate amount of spectral intensity compared to when neither or both DNA templates are present.

However, due to numerous sources of random variation, e.g., spectral noise or the relative efficiencies of individual PCR amplifications, measurements of spectral intensity differ greatly among amplicons, even at loci with known monomorphic genotypes, and therefore do not assort into the three expected genotypic classes. In slab-gel electrophoretic detection systems, the majority of spectral intensity variation results from: (1) the detection procedure, (2) the electrophoresis conditions, (3) the marker assay, and (4) the sample loading volume. While these same components contribute to variability in capillary-based detection systems, their relative contribution to the overall variability may be different. A preferred approach implemented in a preferred embodiment of a method of the invention to distinguish among genotypes relies on minimizing the variance attributed to these noise components by first applying to the original data a series of mathematical transformations and data-processing procedures. Once applied, the spectral intensity of an amplicon, and therefore the relative amount of DNA contained in the amplicon can be accurately quantified ultimately allowing a distinction between homozygote and heterozygote genotypes ascertained by relative proportions of spectral intensity.

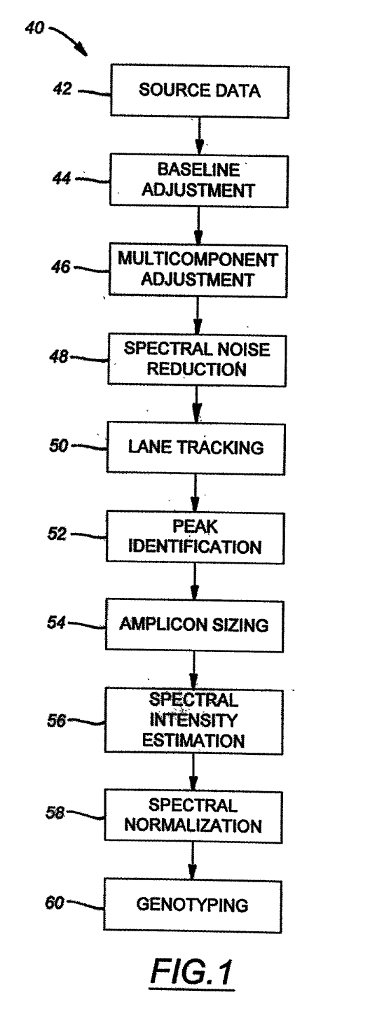

The major processing steps of a preferred embodiment of a method 40 of the invention described herein, such as is preferably employed for one-dimensional or two-dimensional fluorescent detection systems, is shown in FIG. 1 and include at least some of the following steps: (1) collecting source data 42, (2) creating a spectral baseline using the source data and performing baseline adjustment 44, (3) removing spectral overlap by multicomponent adjustment 46, (4) performing spectral noise reduction 48 by attenuating low- and/or high-intensity frequency components of spectral noise, (5) performing lane tracking 50 to identify individual sample lanes, (6) performing amplicon identification 52, (7) performing amplicon sizing 54 to assign a molecular size to each amplicon or fragment, (8) performing spectral intensity estimation 56 for each amplicon or fragment, (9) performing spectral normalization 58 for all amplicons or fragments, and (10) genotyping 60 each amplicon or fragment. In some instances, the order can differ, one or more steps can be omitted, and, one or more steps not listed above can be performed in addition to one or more the above-recited steps.

A preferred embodiment of the method was validated in an insect system, namely Drosophila melanogaster, and an avian system, namely Gallus gallus. Each system had known pedigrees so that genotypes could be unambiguously ascertained for both parental and F1 generations. For example, in the avian system, two highly inbred lines of domestic chicken, Ancona hens and a Rhode-Island Red rooster, were crossed. One such mating produced ten healthy F1 individuals. DNA from each individual was processed, six replicates for each parent and two replicates for each F1, using FAFLP and an embodiment of the method described herein. Eleven loci were examined with nine of the loci being autosomal loci and the remaining two loci being sex-linked loci. The correct genotype was assigned correctly for all test loci examined.

Application of this method to natural populations of birds, namely White-bearded Manakins, Manacus manacus, has also allowed inferences of parentage and dispersal in the subject populations to be drawn that would have been otherwise difficult, if not impossible to achieve. Other applications of a method of the invention include, but are not limited to, systemic pathotyping, population and conservation genetics, disease and quantitative trait locus mapping in humans, plants, or livestock, determination of human identity and genealogical relationships, forensic DNA analysis, and basic population genetic studies on human or non-human biological entities.

### Description of a Preferred Fluorescent fAFLP Procedure

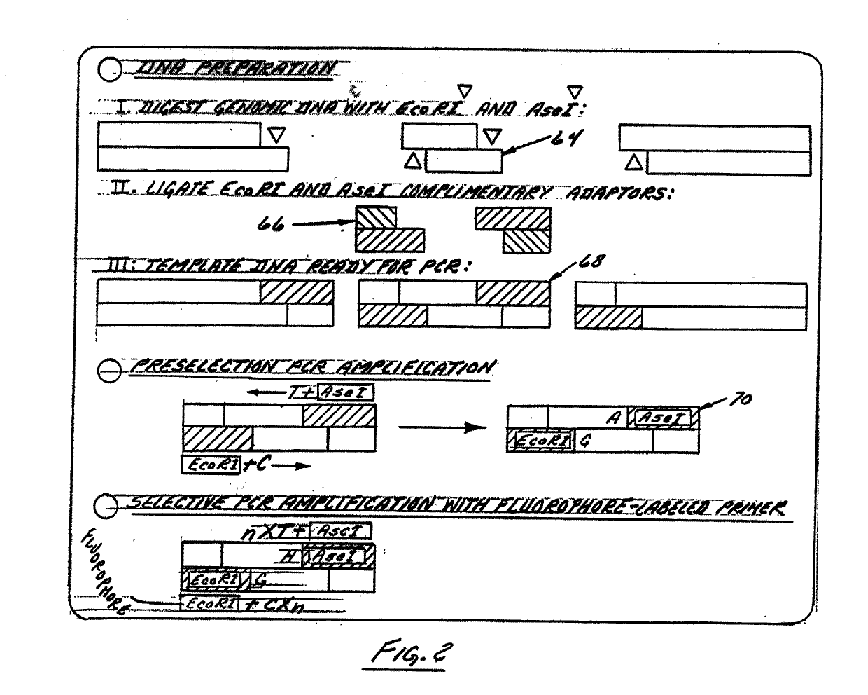

Referring to an example of a fAFLP procedure schematically diagrammed in FIG. 2, a recent improvement in the AFLP process has been the incorporation of fluorescent moieties, commonly referred to as fluorophores, to label individual DNA molecules. This feature allows for convenient detection of labeled DNA fragments 62 and eliminates the need for potentially hazardous radioactive materials, as used in the original AFLP procedure. A fluorophore is attached covalently to the 5′ position of one of the two selective PCR primers, typically the primer homologous to the recognition sequence of the less frequently cutting restriction enzyme (e.g. Eco RI). Following PCR amplification, fluorophore-labeled DNA fragments (amplicons) 62 can be separated and viewed on a machine, typically a detection system, such as an automated DNA sequencer, analyzer, or the like. Currently available automated DNA sequencers typically separate DNA fragments based on differences in size by either (1) slab-gel electrophoresis or (2) capillary electrophoresis. DNA fragment separation by size is useful because fragment size generally corresponds to molecular weight or molecular mobility.

### Description of Electrophoresis of fAFLP-Generated Amplicons

During electrophoresis, amplicons of differing sizes are separated in a matrix that typically is comprised of a polymeric material. In slab-gel electrophoresis, amplicons are separated in a gel matrix comprised of polymerized acrylamide. Operationally, electrophoresis in slab-based gels is two-dimensional as it includes the simultaneous separation of two or more samples in a single gel. The sample being analyzed is applied to the gel across one dimension and is simultaneously separated in the second dimension. For example, as many as 48, 64, or 96, samples can be run simultaneously on ABI slab-gel machines such as the ABI 377, 377XL, or 377-96.

In capillary electrophoresis, DNA fragments are separated in a narrow-bored capillary containing a liquid polymer matrix. Compared to slab-based gel electrophoresis, capillary electrophoresis is operationally one-dimensional as only one sample at a time passes through the capillary. Multiple samples can be processed simultaneously, but each is done so in its own capillary. For example, ABI 310, 3100 and 3700 capillary-based machines respectively contain 1, 16, and 96 capillaries.

An excitation device, typically an argon-ion laser, illuminates the gel matrix at a fixed point, called a stage. When fluorophore-labeled DNA fragments in the gel matrix pass by the stage, the fluorophores are excited by the argon-ion laser and their emission spectra are collected by a detector of the detection system that is sensitive to specific emission wavelengths intrinsic to each fluorophore. The detector collects these emissions at time-dependent intervals generally referred to as scans. Electrophoresis continues for a specified amount of time, generally dictated by the maximum fragment size the operator wishes to detect. More than one scan can be performed during analysis of the matrix.

For clarity, it must also be noted that many automated detection systems can simultaneously detect multiple fluorophores each with emission spectra at distinctly different wavelengths. For example, ABI produces a number of such systems. In contrast to the generalized procedure described above, this allows multiple samples each labeled with a different fluorophore to be electrophoresed simultaneously on a per lane (slab-gel electrophoresis) or per capillary (capillary electrophoresis) basis. Therefore, following the above examples, commonly available ABI machines have the capacity to simultaneously electrophorese 92, 256, or 384 slab-gel samples and 4, 64, or 384 capillary samples.

### Conventional Analysis of fAFLP-Generated Data

Following electrophoresis, fluorophore emission data derived from slab-based gels are stored in computer files commonly called “gelfiles” or “collection” files. To meaningfully analyze the data, lane tracking is required to delimit individual samples. Lane tracking is accomplished by defining a single tracking spline, either by a human operator or using a software algorithm, that bisects each amplicon in a single sample lane. Once each sample and its associated amplicons are delimited by a tracking spline, data associated with each sample can be extracted from the gelfile. This procedure is typically implemented in one of two ways: (1) for each time interval or scan, the recorded value of spectral intensity nearest to the tracking spline is extracted, or (2) for each time interval or scan, the average value of recorded spectral intensity in a defined proximity to the tracking spline is computed and stored. This processing step is performed automatically in capillary electrophoresis, believed to be via software control, as emission spectra for each sample are collected independently.

The end result for conventional processing of photometric data from both slab-gel and capillary electrophoresis is the creation of a sample-specific computer file, commonly called a tracefile, chromatogram, or electropherogram, that, when graphically displayed, depicts the processed signal in two dimensions. Amplicon position is located along one axis, typically the abscissa or X-axis, and relative fluorophore intensity is located along another axis, typically the ordinate or Y-axis. Position corresponds to the scan number, which in turn is proportional to the size of an amplicon.

The presence at a particular position of a high-density of fluorophore-labeled DNA molecules is represented as an increase in spectral intensity above a defined background threshold. Such a region of increased spectral intensity is typically referred to as the spectral peak of the amplicon. The position of the peak, as a finction of scan number, provides a basis by which to estimate the size of the amplicon (gauged by a standard of known DNA fragment sizes) and ultimately the presence or absence of homologous or unique fragments in other samples.

### Description of DNA Sample Preparation for fAFLP Processing

Before any source data can be collected, the chromosomal material (total genomic DNA) for each sample must first be prepared for analysis. FIG. 2 depicts an example of a preferred method of preparing DNA for analysis using fAFLP. In the first step, identified in FIG. 2 by the label “DNA Preparation,” template DNA 64 is obtained by conventional isolation and purification methods. The template DNA 64, e.g., 200 nanograms of purified genomic DNA, is digested to completion with a rarely cutting restriction enzyme, e.g., Eco RI, and a frequently cutting restriction enzyme, e.g., Ase I. Double-stranded adaptors, e.g. 75 picomoles each,

| | |
|---|---|
| 5′-CTCGTAGACTGCGTACCCATCTGACGCATGGTTAA-5′ | (Eco RI) |
| and | |
| 5′-GACGATGAGTCCTGAGTACTCAGGACTCAT-5′ | (Ase I) |

with adaptor ends complementary to the restricted template DNA ends, are ligated with T4 DNA ligase 66 thereby generating a template 68 suitable for PCR, such as is shown in FIG. 2.

Depending on genome characteristics, the restriction-ligation procedure may generate thousands of adapted fragments. For sufficient resolution of each amplicon, i.e., discrimination of individual PCR-amplified amplicons, to occur in denaturing polyacrylamide gel electrophoresis (dPAGE) or capillary electrophoresis, the population (or complexity) of differing templates must first be reduced. This is accomplished in accordance with the original AFLP process by performing two rounds of PCR amplification of a subset of these templates. Each PCR amplification is designed to reduce the complexity of the template population by preferentially amplifying a specific subset of template DNA.

In round one, preselective PCR amplification 70 is performed as illustrated in FIG. 2. During preselective amplification, two primers, e.g., 15 picomoles each, 5′-GACTGCGTACCAATTCC-3′ (Eco RI+C) and 5′-GATGAGTCCTGAGTAATG-3′ (Ase I+G) initially reduce the template population complexity by extending the known adaptor sequence of each primer into the unknown portion of the adapted or ligated template by a single nucleotide base, i.e., Eco RI+C, Ase I+G. Theoretically, each additional nucleotide base extending into the unknown template sequence 70 reduces the complexity of the template population by a factor of 4ⁿ, with n being the number of selective nucleotides on both primers. In the present example, two additional nucleotides are used resulting in the reduction of the template complexity by a factor of 16. The resulting amplicons are diluted, for example, by a factor of 19 with 10 mM Tris-HCl buffer, pH 8.5.

A second round of PCR amplification, labeled “Selective PCR Amplification with Fluorophore-Labeled Primer” in FIG. 2, is performed, for example, using two additional selective nucleotides on the Eco RI primer 5′-GACTGCGTACCAATTCCAT-3′ (Eco RI+CAT, e.g., 1 picomole), and one additional nucleotide on the Ase I primer 5′-GATGAGTCCTGAGTAATGC-3′ (Ase I+GC, e.g., 25 picomoles). This step further reduces the complexity of the template population by a factor of 64 and overall by a factor of 1024. Preferably, the goal in adding selective nucleotides is to reduce the template complexity to a point where non-homologous amplicons (i.e. amplicons of similar size but of different chromosomal origin) do not comigrate with each other. The choice of specific nucleotides to incorporate into the selective primers is made through routine testing and experimentation.

Contrary to the preselective PCR amplification procedure 70, the selective PCR amplification procedure 62 uses an Eco RI+CAT primer labeled with a fluorophore (e.g. 6-FAM) to enable detection of it on an ABI 377XL automated sequencer. Thus, only those amplicons 62 containing an Eco RI restriction site having a fluorophore label will be detected. Those fragments containing only Ase I sites will not be detected as the Ase I primer is not labeled with a fluorophore. In a preferred implementation, selective PCR amplification is performed under conditions of decreasing primer annealing stringency utilizing a procedure known as “touch-down” PCR amplification. The touch-down procedure begins with the fist few cycles of the PCR amplification procedure under conditions of extremely high annealing stringency, for example at 65° C. Such a high annealing temperature insures that only perfect matches of primer and template will result in template elongation. This greatly reduces the potential of spurious amplifications of imperfect primer/template matches. The annealing temperature is gradually reduced, for example by 1° C. each cycle, until the optimal annealing temperature of the primer is reached, for example, 56° C.

### Description of Modifications to the Original AFLP Technique

As typically practiced in the selective amplification phase of the fAFLP/AFLP process, the number of PCR cycles completed often creates conditions where (1) either the PCR primer is prematurely exhausted and/or (2) amplicon production enters a plateau phasewhere amplicon production deviates from exponential expectations and becomes linear. Typically, a plateau phase is entered after completing approximately 30 to 35 PCR cycles. The point at which any amplicon enters a plateau phase is dependent on: (1) the initial concentration of template DNA, (2) the concentration of both PCR primers, and (3) the number of selective PCR cycles.

Referring to FIG. 3, the plateau-effect phenomenon of PCR amplification may directly prevent discrimination of any relative differences in DNA concentration among amplicons. As a consequence, inferences concerning relative differences in the original amount of template DNA, as reflected in the PCR-amplified amplicon, cannot generally be made. For example, consider any amplicon generated from a 1× and 2× difference in initial template DNA concentration (cf. the difference in expected template concentration when comparing heterozygote and homozygote present genotypes), such as is depicted in FIG. 3. There is a theoretically approximate 7 cycle lag behind which an amplicon derived from the lesser concentration of DNA (e.g. 1×) will enter the plateau phase compared to an amplicon derived from a greater concentration of DNA (e.g. 2×). If both amplicons have already entered the plateau phase, any method that quantifies the DNA concentration in amplicons, either directly or indirectly, will be unable to infer relative differences in the original template DNA concentration. This is a critical problem as heterozygote genotypes can actually appear as homozygote genotypes and be misidentified as such (FIG. 3).

To help minimize this problem, the original AFLP protocol is modified in the present invention by (1) increasing the amount of both PCR primers in both amplification steps and by (2) adjusting the number of PCR cycles to restrict entry into the plateau phase preferably maximizing the relative difference in amplicon concentration between homozygote and heterozygote genotypes. In a preferred implementation, amplicons generated in the pre-selective PCR amplification phase, such as by using 15 picomoles each of unlabeled primer, are diluted with 10 mM Tris-HCl, pH 8.5, and adjusted to a concentration of 20 nanograms per microliter. Five microliters of this dilution provides, on average, 100 nanograms template for the selective PCR amplification step. Primer concentrations in the selective PCR amplification step are adjusted to 5 picomoles for the fluorophore-labeled primer and 25 picomoles for the unlabeled primer. Following initial touch-down PCR amplification protocols, the selective PCR amplification phase continues for an additional 17 cycles. Our empirical analyses have indicated that these conditions provide an environment where the vast majority of amplicons will not enter the plateau phase, thus providing a preferred physical criterion by which to accurately and precisely infer differences in initial template DNA concentrations. This ultimately helps to ensure that an accurate assignment of genotype can be made for each amplicon.

Using the aforementioned template preparation process, different primer pairs, which differ by, for example, a single nucleotide base, will amplify a different subset of adapted DNA templates. By using combinations of primers with different selective nucleotides, a series of fAFLP amplifications will amplify DNA templates (alleles) from a large number of loci in the genome. With the ability to control the number of selectively amplified templates, an optimal number of DNA amplicons may be generated, thereby avoiding complications intrinsic to the fAFLP process itself. For example, excessive DNA template amplifications are avoided. This problem usually produces uninterpretable smears of amplicons when electrophoresed in a gel. In cases where smears are not observed, excessive template amplication often results in unacceptable levels of amplicon co-migration, a phenomenon where amplicons derived from non-homologous loci migrate in a gel at the same molecular weight. Moreover, adjusting the number of PCR cycles in the selective amplification step restricts amplicon production to the log-linear phase, thus preserving a distinction of any differences in initial DNA concentrations.

### Description of the Preparation of Amplicons for Gel Electrophoresis

In a preferred implementation, amplicons containing fluorophore-labeled DNA fragments generated during selective PCR amplification with the aforementioned modifications are purified over Sephadex columns, preferably using SEPHADEX G-75 marketed by Sigma-Aldrich Chemical Corporation of St. Louis, Mo. This step preferably removes unincorporated primers (both labeled and unlabeled) and nucleotides as well as contaminating salts that may interfere with the electorphoresis procedure. The amplicons are then separated on a dPAGE automated sequencer using a slab gel made with a 5% acrylamide mix, preferably using a 5% Long-Ranger® acrylamide mix marketed by BioWhittaker Molecular Applications, Inc. of 191 Thomaston Street, Rockland, Md. A standard containing known sizes of DNA fragments, such as ILS-600, marketed by Promega Corporation of 2800 Woods Hollow Road, Madison, Wis., is included in each sample to control for any lane-to-lane amplicon migration variability and thus provide a basis from which to determine the sizes of the size-unknown amplicons. The DNA size-standard is labeled with a different fluorophore, e.g. ROX, than the fAFLP-generated sample amplicons and allows both to be easily distinguished.

### Generation of Source Data

In a preferred implementation, amplicons are separated and visualized on an automated DNA sequencer manufactured by ABI. A currently preferred model of a slab-based automated DNA sequencer is the ABI 377XL. The ABI 377XL separates and records the presence of fluorophore-labeled DNA fragments at a fixed point in a polyacrylamide gel using a charged coupled device (CCD) detection apparatus. Other instruments may also be used including those that use a complementary metal oxide device (CMOS) to detect spectral emissions.

In one preferred implementation using an ABI 377XL automated sequencer, the upper buffer chamber is loaded with 0.70×TBE buffer and 1.0×TBE buffer is loaded in the lower chamber. Heat denatured samples are loaded onto the gel and electrophoresed at reduced power, typically 10 Watts, for two minutes. A solution of 10×TBE is then added to the upper buffer chamber to achieve a 1×TBE concentration followed by electrophoresis at normal power, which typically is about 40 Watts.

This procedure, known as a “water load” enhances fragment stacking, which is a phenomenon where differently sized DNA fragments orient themselves according to size upon initial entry into the polyacrylamide gel matrix. This has a marked effect of reducing the apparent “broadness” of a group of identically sized DNA fragments (an amplicon) as they migrate through the gel, thereby increasing overall amplicon resolution. The electrophoresis power is set preferably to a limiting 40 Watts, which appears to optimize amplicon cohesiveness by minimizing excessive diffusion (observed at very low power) and by minimizing physical distortions due to sieving effects (observed at very high power). For a 36 cm gel plate, a seven-hour runtime detects amplicon sizes of approximately 20 to 1200 bases.

Other electrophoresis conditions may also be used. For example, electrophoresis power can be adjusted such that it differs from that discussed above. Samples can be loaded with 1.0×TBE buffer in both chambers with or without a two-minute electrophoresis pre-run. Other buffers differing in composition and concentration may alter the electrical characteristics of the system and may beneficially affect the electrophoretic behavior of the DNA fragments. Routine testing and experimentation can be used to determine whether one or more of the aforementioned parameters affect optimal base stacking and amplicon migration.

Separated fluorophore-labeled DNA fragments (amplicons) are visualized by exciting the fluorophore with an external emission source and recording the intensity of fluorescence emitted therefrom. These data are collected and recorded in real time during the length of the electrophoresis session. Typically, the fluorophore emission data is stored in a computer file as detailed below. For example, ABI Collection Software v2.5, used on an ABI 377XL automated sequencer, creates a gelfile that contains a complete record of the raw fluorophore emissions for an entire electrophoresis session.

If desired, fluorophore-labeled DNA fragments (amplicons) can be separated and visualized on different types of imaging devices. Such devices include, but are not limited to: the NEN® Global IR² DNA Sequencer System from LI-COR, Inc. of Lincoln, Nebr., which detects near-infrared fluorescent dyes, the ALFexpress II DNA Analysis System from Amersham Pharmacia Biotech, of Piscataway, N.J., and the FluorImager®, which is a multi-purpose fluorescent sample imager for multiple-color analysis from Molecular Dynamics, a company of Amersham Biosciences of Piscataway, N.J. Fluorophore-labeled DNA fragments can also be separated and detected using instruments that employ capillary electrophoresis technology, such as is used in the ABI 310, ABI 3100 and ABI 3700 Genetic Analyzers. Each of these devices has their own unique operating procedures. Adjustments to the aforementioned slab-gel or capillary-based electrophoretic procedures may be necessary to accommodate the requirements of each detection technology.

### Methodology of Data Processing Procedures Used in Genotyping of fAFLP-Generated Amplicons

To accurately and precisely distinguish among the three possible genotypic classes of amplicons generated by the fAFLP process, the resultant source data containing the actual fluorophore emission data, henceforth termed photometric data, must first be processed in accord with the invention detailed below. In its original form, the photometric data contains various sources of variability and error, typically in the form of noise, which negatively affects accurate quantitation of spectral intensity, and therefore the amount of DNA in any amplicon. In slab- and capillary-based detection systems, the majority of random variation observed in measurements of DNA concentration is believed to result from differences in: (1) the marker assay, (2) the sample loading volume, (3) the electrophoresis conditions, and (4) the detection procedure. These four sources of variability are set forth in Equation I below,

σ_{Total}²≅σ_{Marker assay}²+σ_{Loading volume}²+σ_{Electrophoresis}²+σ_{Detector}² (Equation I)

where

σ²_{Total} is the total estimated variability in the source data;

ρ²_{Marker Assay} is the total estimated variability due to variations in the marker assay;

σ²_{Loading Volume} is the total estimated variability due to variations in sample loading volume;

σ²_{Eectrophoresis} is the total estimated variability due to variations in electrophoresis conditions;

σ²_{Detector} is the total estimated variability due to variations in detector operation.

A present method of distinguishing among genotypes in amplicons generated from fAFLP assays in accordance with a method of the invention relies on first minimizing the variance attributed to these components by applying to the original data a series of mathematical transformations and image-processing procedures. Once applied, the relative amount of DNA in any amplicon, as reflected by spectral intensity, can be accurately estimated and normalized across multiple samples. This procedure enables an inference to the original number of DNA templates (alleles) originally present at any locus thus allowing a determination of the genotype of the amplicon.

Briefly, the procedure consists of applying baseline, multicomponent, and Fourier-based noise reduction procedures to reduce variation attributed to the detection and electrophoresis apparatus. Sample amplicon sizes are estimated using a statistical model designed to enhance the predictive accuracy and precision of sizing curves generated from size-standardized DNA fragments. Estimation of spectral intensity of each amplicon is based on a statistical model derived from characteristics of single-stranded DNA in denaturing electrophoretic gels. This model provides an accurate means by which to measure the amount of DNA in each amplicon. Normalization of these estimated spectral intensities is accomplished by using a monomorphic locus present in the population of samples. This type of normalization provides a basis for minimizing variation attributed to the marker assay itself and unequal sample loading volumes.

A purpose of this invention is to apply one or more of the procedures disclosed herein in a manner such that direct comparisons of spectral intensity, and therefore DNA abundance, can be made among amplicons present at any locus. In doing so, a distinction as to whether the amplicon was originally derived from one or two alleles can be inferred. With this information, a distinction between homozygous and heterozygous genotypes can be made for any amplicon. The method described herein is applicable to a broad class of genetic markers and marker-detection technologies and can be achieved using source data comprising a one- or two-dimensional vector of spectral intensities sampled at regular time intervals.

Although no reasons are known to exist why the method of the invention would not work with other marker assays, detection systems, aneuploid or polyploid organisms, the description set forth below has been limited to DNA-based, fAFLP-generated amplicon assays in diploid organisms visualized on an ABI 377XL slab-gel automated sequencer for the sake of describing at least one preferred implementation of the method. Where practical, it is identified herein what procedures are applicable to specific technologies and what algorithm is used to perform the particular procedure. The order in which each procedure is discussed herein reflects a preferred order of its application.

### Source Data Preprocessing

When the source data contains information in addition to that required for genotype discrimination, it may be necessary to first preprocess the source data using an algorithm preferably implemented in software that extracts the pertinent data. For example, when using ABI automated sequencers, ABI Collection Software, e.g., version 2.5, is used to record raw fluorophore emissions from the detection apparatus. The data comprising the raw fluorophore emissions is stored in a file that has a proprietary format either as: (1) a gelfile (or collection file), such as for slab-based machines or (2) a sample file (or tracefile), such as for capillary-based machines and for at least certain types of source data derived from some slab-based machines. Data from these files, or files such as these, contain the actual source data. If desired, the source data can also constitute data outputted directly from any such machine or other suitable device in real time and need not be pre-stored in a file.

In one method of preprocessing, source data generated from an automated sequencer or other like device, source data from a gelfile or sample file are used as input that is processed to locate, extract, and, if desired, manipulate at least some of the data contained therein. If desired, two-dimensional source data, such as photometric data from a gelfile, can be processed to obtain one-dimensional source data that resembles or is similar to data contained in a sample file. As previously mentioned, such source data can come from a slab-based DNA sequencer such as an ABI automated sequencer. ABI-derived data or like data from other types of sequencers or fluorescence imaging devices that produce or contain photometric data can also be used as source data. Hence, the term “gelfile” is used herein to generically indicate two-dimensional source data and “sample file” is used herein to generically indicate one-dimensional source data.

Source data are typically stored in a computer readable file. Photometric data are processed by first locating it within the source data, extracting it, such as by copying it, and, if desired, subsequently formatting it in a manner that preferably simplifies the manner in which it is stored. The extracted and/or processed photometric data can be written to another output file for subsequent use or can be used without being first written to an output file. Since not all of the previously stored data are extracted during processing, the resultant output file is often smaller than the original file containing the source data.

Depending on how the source data is organized, additional information other than the photometric data itself may be present. This information can be used in locating and identifying photometric data as well as other relevant photometric-related data. Once located, the photometric data are extracted along with any other related data desired or deemed necessary. The data can then be reorganized to a format that is preferably more easily manipulated and inspected or used as needed.

In one preferred implementation, photometric data stored in an ABI-generated gelfile or sample file, in a form representing the intensity of recorded fluorophore emission data as integer values between 0 and 8191, is extracted. This data may be rescaled into integer values ranging from 0 to 255. Although rescaled, this new range of integer values still represents the relative intensity of fluorophore emission data originally collected. An advantageous result of this procedure is that the rescaled data are easier to manipulate such that it can also be used by other algorithms, such as those found in graphics software, graphics related software, statistical analysis software, gene fragment analysis software, data visualization software, as well as in other types of software. For example, processed and resealed photometric data can be analyzed with freely available gene fragment analysis software, e.g., Cross Checker, which is available from the Department of Plant Science, University in Wageningen, the Netherlands.

In one preferred implementation where a PC or PC-compatible computer is used, it may be necessary to convert the source data into another byte order, e.g., LITTLE_ENDIAN, before performing any preprocessing procedures. It may also be necessary to convert the source data into another byte order, e.g., BIG_ENDIAN, when UNIX operating systems or Macintosh computers are used to preprocess gelfiles or sample files originally stored on a PC or PC-compatible computer. Software modules are available that easily convert data to and from LITTLE_ENDIAN and BIG_ENDIAN format. One such module is available from the Medical Research Council as part of the Staden Package at the MRC Laboratory of Molecular Biology, Hills Road, Cambridge, United Kingdom. Such a conversion may be needed when going from one type of platform to another. However, such conversion or reformatting may not always be needed.

The presently preferred source data preprocessing procedure locates, obtains and extracts the photometric information contained within the source data, e.g., either a gelfile or sample file. Header information included in the source data being preprocessed is examined to determine where this information resides.

### Structure and Organization of ABI Gelfiles

Where the source data is an ABI gelfile, it is assumed that the gelfile begins with one or more specific file identifying characters. For example, ABI gelfiles derived from ABI Collection Software v2.5 begin with 11 concatenated header records, which vary in length depending on the number of channels scanned during an electrophoresis run. For example, depending on options specific to individual machines, the ABI 377 series has the capability to scan 194, 388 or 480 channels. Record lengths (LEN_RECORDS) for these scan options are known to be 407, 795 and 979 bytes respectively.

The initial eleven individual header records are designated by two single ASCII characters beginning at byte [(LEN_RECORDS*i)] and byte [(LEN_RECORDS*i)+11], where i=header record {0, 1, 2, . . . 10}. Immediately following the ASCII character at header byte [(LEN RECORDS*i)] is a numerical value designating LEN_RECORDS after subtraction of a portion of the record header and checksum bytes (see below).

Any information stored within these initial eleven headers appears to begin at byte [(LEN_RECORDS*i)+17]. At present, only 5 of the 11 header records have a known function, including header: (1) ABI collection software version, (3) ABI machine type, (5) Number of referenced data blocks (NUM_RECORDS), (6) offset to the primary photometric data block, and (7) length of the primary photometric data block.

In contrast to the organization of the initial eleven record headers, all data records associated with actual photometric data begin with the ASCII character “>” and are followed by a numerical value designating the amount of data, in bytes, stored in each data record after subtracting a portion of the record header, which currently is four bytes in length, and a data checksum, which currently is two bytes in length. The three possible initiators for 194, 388 and 480 channel ABI 377 data records are >401, >789, and >973 respectively. Recorded fluorophore emission data is stored in the primary photometric data block as binary data representing two-byte integers with corresponding decimal values ranging from 0 to 8191.

Fluorophore emission data is recorded in a channel by filter by scan fashion. Each channel can be considered as a column vector of data values, each representing divisions across the width of the gel or capillary, with scans represented as rows across the column vectors. Each filter comprises a distinct range of detectable wavelengths emitted by a particular set of fluorophores. Most ABI automated sequencers can concurrently detect 4 fluorophores. Thus, the photometric data is a matrix of two-byte integers representing fluorescence intensity at a particular channel by row position, sorted by filter number.

The first photometric data record is located at a static offset from byte [0] in 194, 388, and 480 channel gelfiles and is 4477, 8745, or 10769 bytes, respectively. Also, depending on the number of channels, the photometric data contains length-records (LEN_RECORD) of 407, 795, or 979 bytes that include a 17-byte sub-record and a two-byte checksum. Within each of these records, the portion of the record allocated to actual photometric data is 388, 776, or 960 bytes, respectively.

Each data record contained in the primary photometric data block contains 10 fields: (1) RECORD_START, (2) DATA_BYTES, (3) an ASCII “space” character, (4) GEL_VOLTAGE, (5) GEL_CURRENT, (6) GEL_TEMPERATURE, (7) FILTER_NUMBER, (8) TRAVERSE_COUNTER, (9) SCAN DATA, and (10) SCAN_CHECKSUM.

The RECORD_START field contains the initiator for a new scan line (>) followed by a three-byte field, DATA_BYTES, which indicates the total record bytes remaining after subtracting the number of data bytes associated with the RECORD_START, DATA_BYTES and SCAN_CHECKSUM fields. The two-byte fields of GEL_VOLTAGE, GEL_CURRENT, and GEL_TEMPERATURE provide values of gel voltage in units of volts/10, gel current in units of milliamperes, and gel temperature in units of degrees Celsius, respectively, at the initiation of each new scan.

The format in which ABI stores fields 4, 5, and 6 can be confusing. Recorded values are apparently stored as integer-coded two-byte values. However, it is the corresponding hexadecimal representations of these bytes that are actually interpreted. For example, GEL_VOLTAGE is stored as an integer-coded, two-byte value. An example of such a value representative of GEL_VOLTAGE is [61 38]. This corresponds to the ASCII values [a] and [8]. These two ASCII values are concatenated to form a hexadecimal value of 0xa8. The decimal equivalent of 0xa8=168. As this value is in the units of Volts/10, the actual voltage in this example is 1680 Volts. Similarly, for example, GEL_CURRENT: [31 61]=ASCII [1][a]=0x1a=26 mAmps; for GEL_TEMPERATURE: [33 31]=ASCII [3][1]=0x31=49° C.

The field FILTER_NUMBER refers to the filter numbers assigned to detect emissions from particular fluorophores. Where four fluorophores are used, filter numbers 0, 1, 2 and 3 are used. The TRAVERSE_COUNTER field records the cumulative traverses of the laser across the gel. One scan corresponds to 4 traverses of the laser as one traverse is performed for each filter number. The TRAVERSE_COUNTER initializes at 1 and resets to 1 after 2\hat{ }16 traverses of the laser. Immediately following is the SCAN_DATA field, which actually contains the photometric data stored as two-byte integers. Lastly, a two-byte field, SCAN_CHECKSUM, appears to be some sort of checksum resulting from modulus arithmetic on the SCAN_DATA. The form of the checksum is presently unknown.

### Extraction of Photometric Data from Gelfiles

In a preferred implementation of a procedure for extracting photometric data, two-dimensional source data, typically in the form of a two-dimensional matrix of raw digital data, is processed. Such is typically the case where slab-gel electrophoresis is used and a gelfile is created. Photometric data in the source input is processed by locating it and extracting it, preferably to another output file for subsequent use.

In a preferred implementation, an extraction algorithm processes any gelfile derived from an ABI 373 automated sequencer, including units having 194 and 388 channels, and an ABI 377 automated sequencer, including units having 194, 388, and 480 channels. First, the expected eleven header records are checked for the correct content and offsets. Depending on the particular model of automated sequencer, if the initial header does not exhibit the aforementioned features associated with its respective machine model, an error message preferably is generated. Next, the offset to the photometric data and the length of the photometric data is retrieved from the header records 6 and 7 described above. The photometric data is extracted and saved to a new file. For example, in one preferred implementation, a gelfile of length 76,901,936 bytes, derived from an ABI 377XL 388 channel automated sequencer, was processed. Beginning at an offset of 8745 bytes from byte [0] and extracting 16011 scans* 4 data rows per scan* 766 bytes of photometric data per row resulted in a new file, containing only photometric data, of length 49,698,144 bytes.

The structure of the new file containing the extracted photometric data is greatly simplified compared to the original format. In one preferred implementation, a single line of two, five digit numerals, separated by a space, indicate the number of channels and the number of scans. This information is immediately followed by the extracted photometric data written as rows of two-byte, binary-encoded integer data arranged in a scan by filter by row fashion. For example, following the above criteria, this organization would be represented as:

00388 16011
scan 1:filter 0:row 1
scan 1:filter 1:row 2
scan 1:filter 2:row 3
scan 1:filter 3:row 4
scan 2:filter 0:row 5
. . .
scan (n):filter 0:row (4n-3)
scan (n):fllter 1:row (4n-2)
scan (n):filter 2:row (4n-1)
scan (n):filter 3:row (4n-0)

Note that in this example, the newly created file of extracted photometric data will be 12 bytes larger than expected from the previous formula (i.e. 49,698,156 bytes). This is due to the addition of the 12-byte channel number by scan number header information.

In addition to extracting the raw photometric data contained in a gelfile, the extraction method preferably also extracts the machine runtime parameters of voltage, current, and temperature, and outputs simple summary statistics of the photometric data including the minimum and maximum relative fluorescence intensity (RFI) in each filter along with a histogram of RFI values observed in each filter. This output can be and preferably is used in the optimization of fluorophore concentrations in FAFLP reactions and to detect any anomalous machine behavior.

### Structure and Organization of ABI Sample Files

Where the source data is an ABI sample file, it is assumed that the sample file begins with one or more specific file identifying characters. For example, ABI sample files begin with a single 128-byte file header. The first four bytes of this header (i.e., 0x41 0x42 0x49 0x46: “ABIF”) designate the file as an ABI processed sample file. In its current implementation, a single statically located 28-byte record, offset six bytes from the beginning of the sample file, provides information about the structure and location of dynamically located blocks of data. This “table of contents” or index contains a plurality of records that each provide information about the various types of data stored in the sample file. The index is made up of a group of records each referred to as a “FLAG”. Each FLAG record of an ABI sample file is also 28 bytes long and contains eight fields: (1) FLAG_HEADER, (2) FLAG TAG_VALUE, (3) DATA_TYPE, (4) RECORD_SIZE, (5) NUM_RECORDS, (6) LEN_RECORDS, (7) DATA_VALUE, and (8) an unknown field. The term FLAG is used herein in the context of being a type of record and therefore is used in a manner that is different from its conventional usage in computer parlance. Each FLAG contains identifiers that describe the type of data to which the FLAG points to or contains. Typically, the associated data record is stored elsewhere in the tracefile, the location of which is identified somewhere in the FLAG record.

The FLAG_TAG_VALUE consists of an integer value that distinguishes among multiple FLAG records that are identically named. For example, where a FLAG record identifies or points to photometric data, or to its location, the FLAG_TAG_VALUE field indicates which fluorophore was used or analyzed as well as whether the photometric data are raw or otherwise processed, for example, with proprietary ABI analysis software such as GeneScan. More specifically, because there are several records, typically twelve, that contain the FLAG “DATA,” associated FLAG_TAG_VALUEs 1-4 indicate that the associated FLAG respectively identifies or points to raw, e.g., unprocessed, spectral data for fluorophores 1-4, and associated FLAG_TAG_VALUE 9-12 indicate that the associated FLAG respectively identifies or points to ABI processed spectral data for fluorophores 1-4.

The DATA_TYPE field indicates the structure or type of data that is stored in the location to which the associated FLAG points to or identifies. For example, DATA_TYPE 0x0004 means that the data stored in the location to which the associated FLAG points to or identifies is a two-byte integer. In a current implementation, all raw or processed ABI photometric data is stored as DATA_TYPE 0x0004.

The RECORD_SIZE field indicates the length, in actual bytes, of the referenced DATA_TYPE stored in the location to which the associated FLAG points to or identifies. For example, where the data are of DATA_TYPE 0x0004 the RECORD_SIZE field will hold the value 0x0002, i.e., a two-byte integer).

The NUM_RECORDS field holds a value that indicates how many entries of a particular DATA_TYPE is present starting at the location to which the associated FLAG points to or identifies. For example, where an associated FLAG points to or identifies a location in the tracefile at or after which 100 (0x64) observations of fluorescent intensities are stored, the value of the associated NUM_RECORDS field will be (0x64).

The LEN_RECORDS field holds a value that denotes the total length, in bytes, of all NUM_RECORDS associated with a particular DATA_TYPE and FLAG. NUM_RECORDS can also be characterized as denoting the total length, in bytes, of NUM_RECORDS of referenced data. For example, where the particular data associated with a particular FLAG record has DATA_TYPE 0x0004 and NUM_RECORDS has value (0x64), the value of LEN_RECORDS will 0x00C8 (i.e., 2 bytes per record*100 records=200 bytes).

### Extraction of Photometric Data from ABI Sample Files

In a preferred implementation, an extraction method is used to process ABI-generated sample files to extract the photometric data contained therein. First, the header content is checked for the correct content and offsets: byte 1-4 must contain the values 0x41 0x42 0x49 0x46 (“ABIF”) and must include a 28-byte FLAG index record located at an offset of six bytes from byte [0]. If the initial header does not exhibit the aforementioned features, an error message preferably is generated. Next, each FLAG record is sorted and accessed to process record information. In a preferred implementation, only available FLAGS and associated records are sorted and displayed. The pointer index, which is a table of FLAG records, is consulted to determine the location of the photometric data contained in the sample file. For example, where the file is an ABI-generated sample file, each FLAG record that points to photometric or photometric related data, such as certain DATA [FLAG] records, is identified. Using the location associated with each such pointer, the DATA_TYPE, RECORD_SIZE, NUM_RECORDS and LEN_RECORDS information is used to specify and extract the photometric data to a new output file containing only the extracted data. This data is retrieved because it contains the actual photometric data in the form of preferably raw and/or processed ABI fluorophore emission data. Data contained in other sample files can also be selectively located and extracted in this same manner.

### Description of the Genotyping Process

In describing the genotyping process, the forthcoming procedures are each presented with reference to a data vector as operationally defined by the dimensionality of the source data. For example, one-dimensional source data, such as photometric data obtained from a processed sample file, consists of a one-dimensional vector of photometric data. A two-dimensional data source, such as photometric data obtained from a gelfile, consists of a series of adjacent one-dimensional vectors that correspond to channels of photometric data.

Operationally, for some procedures described herein, the two-dimensional data is reduced to a series of one-dimensional vectors by applying a procedure to each data channel separately. For example, in the procedures of baseline adjustment, multicomponent correction, and spectral noise reduction, a one-dimensional form of the procedure is applied successively to each data channel, even though the original source data may have been two-dimensional. This does not mean two-dimensional applications of these procedures do not exist, or possibly even warrant merit. In fact, the use of a two-dimensional approach to estimate spectral intensity, e.g., source data from gelfiles, is well suited for being practiced using a preferred implementation of a method of the invention despite the fact that a one-dimensional approach, e.g. sample files, can be used. In a currently preferred implementation, a one-dimensional approach is preferred as it (1) provides excellent results and (2) costs less in terms computational time required to complete certain calculations.

### I. Baseline Adjustment

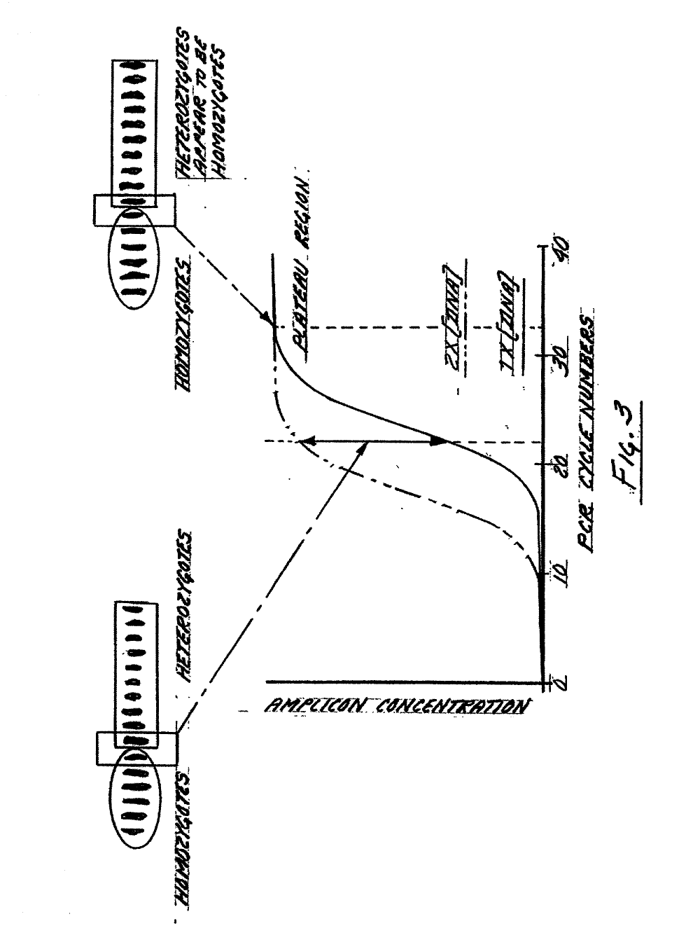

During operation of an electrophoresis apparatus, a detector records the presence and, within the limitations of the detector, the relative intensity of emissions of fluorophore-labeled DNA fragments as they pass by a detection area. While the signal that the detector produces or outputs can be a digital signal, it typically is an analog signal that corresponds to the intensity of the fluorescence. The analog signal is subsequently converted to digital data. It is this digital data that comprises the source data.

Where an excitation device is used, its output ideally produces a response only from fluorophore-labeled DNA fragments in the polymer matrix that is read by the detector. For example, a laser-based excitation device causes fluorophores attached to DNA fragments to fluoresce and the detector is designed to detect that fluorescence. However, as is exemplified by FIG. 4, components other than fluorophores themselves may also fluoresce, therefore confounding the signal associated with the fluorophore-labeled DNA fragments. This phenomenon often leads to signal variability or error, typically in the form of spectral noise, which manifests itself as unwanted components present in the resultant detected signal.

More specifically, anything irradiated by the excitation device may produce some amount of response that can contribute to spectral noise. Where fluorophore excitation is intended to cause fluorescence, the polymer matrix that holds the DNA fragments itself can produce some aberrant spectral signals. Other components can also contribute additional spectral noise. For example, the glass-plate material holding the matrix, debris on the glass plates, capillary tubing, as well as other material in and around the matrix, can contribute noise. In most cases, the presence of this spectral noise manifests itself in an additive manner, i.e., an offset, to a signal that is only supposed to correspond to desired fluorophore emissions.

Such offsets, generally identified by reference numeral 72 in FIG. 4, have been shown to generally possess relatively uniform and constant amplitude. Where such an offset is an issue, a baseline adjustment preferably is performed to determine an offset correction that removes it completely or at least a substantial portion of it. The remaining component therefore consists primarily of the signal contributed by desired fluorophore emissions. Ideally, when baseline adjustment is complete, the signal intensity of all of the photometric data originates at about the same level and enables a better comparison of relative spectral signal intensity.

In a preferred implementation of a procedure for performing baseline adjustment, data from the electrophoresis apparatus are outputted for a certain amount of time and the data retained as source data. To the extent needed, photometric data is extracted and this data is processed to adjust its baseline.

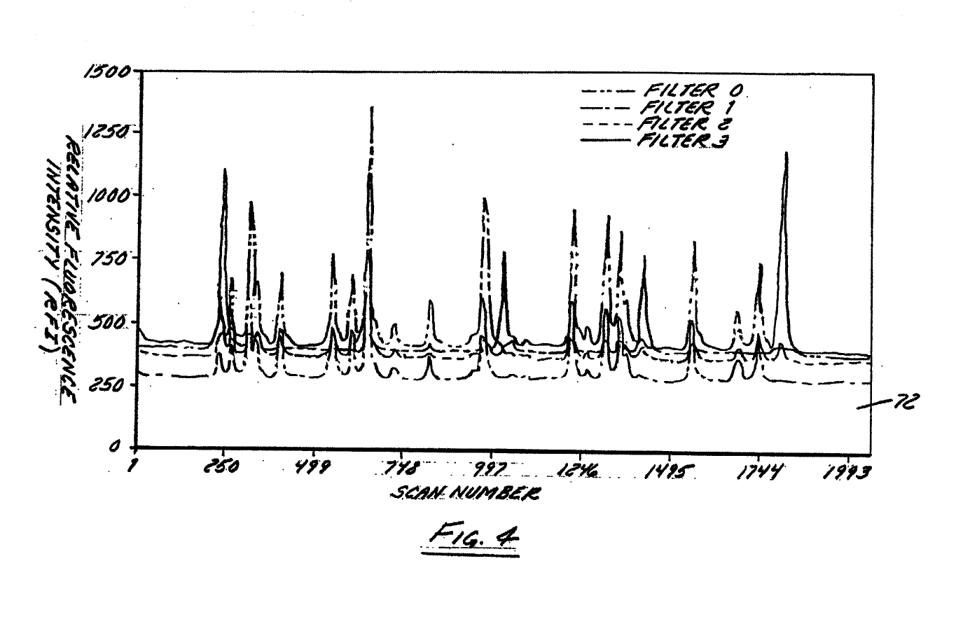

Still referring to FIG. 4, each data point of a particular scan and filter that is processed to adjust its baseline is defined by a position that corresponds to its scan number along the X-axis, and a value indicating relative spectral intensity (RFI), that corresponds to its RFI along the Y-axis. For example, referring to FIG. 5, the baselining procedure first groups data points into windows, W₁, W₂, and W₃, of data points each having a fixed number of data points. In the preferred implementation depicted in FIG. 5, each window includes 50 data points, but each window can be larger or smaller depending on the degree of baseline removal desired.

For each window, a search is performed to determine the data point having the lowest value of spectral intensity or RFI. The slope of a line 74 between the lowest value of one window, W₂, and the lowest value of the next adjacent window, W₃, is determined, preferably by calculation. Next, the magnitude of baseline offset of each data point between the two window minima is computed. An offset value is then computed for each data point between the lowest values of spectral intensity of one window, W₂, and up to the data point with lowest value of spectral intensity of the next adjacent window, W₃. The interpolated baseline offset is subtracted from the value of spectral intensity associated with each data point. In a preferred implementation, a baseline offset is calculated for each data point using multipoint linear regression, and the calculated offset is subtracted from the value of spectral intensity of each data point.

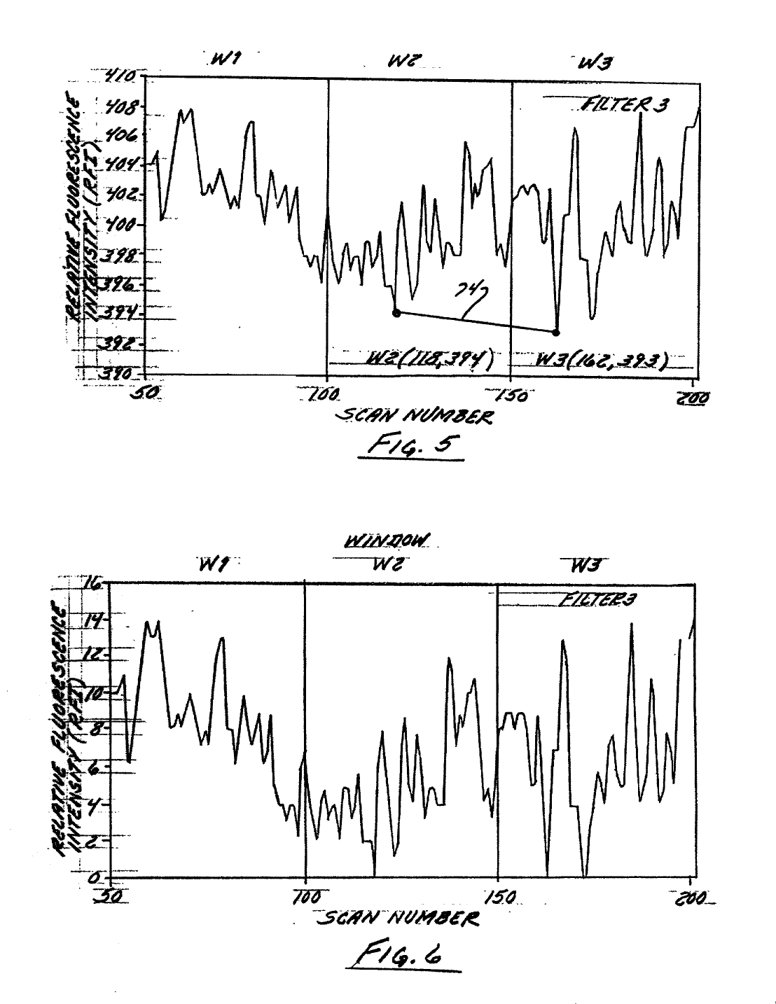

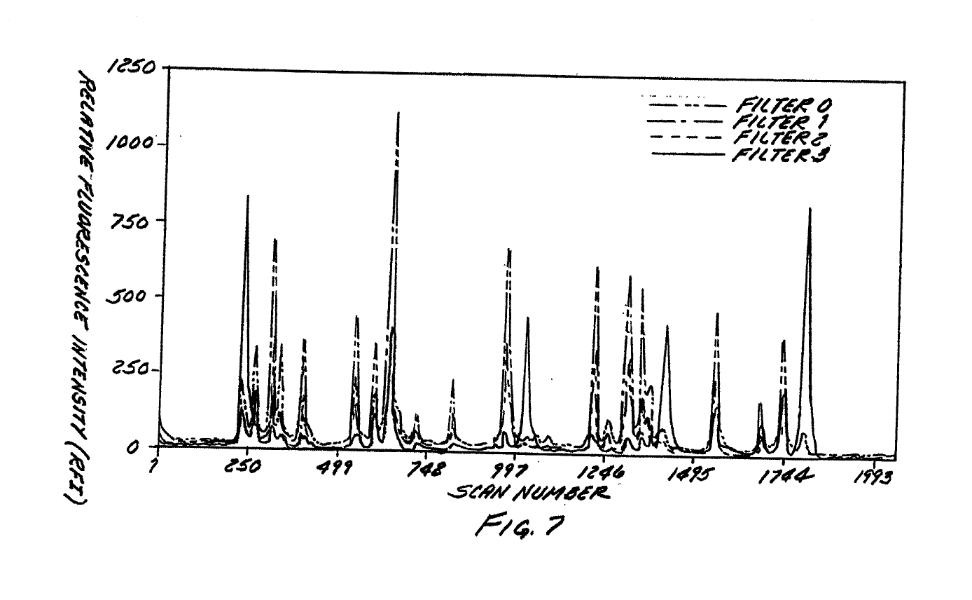

Referring to FIG. 6, after the offset is subtracted, the values of spectral intensity preferably are shifted to a common baseline. Referring to FIG. 7, this is done for each scan and filter. An advantage of the present method of performing a baseline adjustment on the data is that the baseline is implemented dynamically. It is therefore capable of adjusting the baseline in response to changing spectral noise conditions. A preferred implementation of determining baseline-offset correction is presented in more detail below.

In single- or multi-component fluorophore-based detection systems, signal variability due to detection process often results in the accumulation of substantial spectral noise visually discernible as a signal offset from a baseline of zero spectral amplitude. To dynamically remove this offset, first consider a one-dimensional vector of spectral amplitudes (intensity responses) Yᵢ, i=1, 2, . . . , n each indexed with corresponding predictor positions Xᵢ, i=1, 2, . . . , n . This vector is partitioned into W=n/l windows, where l represents the minimum level at which baseline-offset variability is removed (typically 50 to 100 predictor positions). In each window Wⱼ, j=1, 2, . . . , n/l, the minimum spectral intensity response Wⱼ(Yᵢ^{*}) and its corresponding predictor position, Wⱼ(Xᵢ^{*}) is identified. The slope, λⱼ, between minima in successively adjacent windows is then computed,

$${W}_{j} ( {λ}_{j} ) = \frac{{W}_{j + 1} ( {Y}_{i}^{*} ) - {W}_{j} ( {Y}_{i}^{*} )}{{W}_{j + 1} ( {X}_{i}^{*} ) - {W}_{j} ( {X}_{i}^{*} )} j = 1 , 2 … , n / l . \qquad ( Equation II )$$

Next, the magnitude of baseline offset, γⱼ^{*}, for each Wⱼ(Xᵢ^{*}, Yᵢ^{*}) is computed,

Wⱼ(γⱼ^{*})=Wⱼ(Yᵢ^{*})−Wⱼ(λⱼ)[Wⱼ(Xᵢ^{*})]. (Equation III)

Using estimates of Wⱼ(λⱼ) and Wⱼ(65ⱼ^{*}), offset values, γᵢ, for each response Yᵢ between Wⱼ(Xᵢ^{*}, Yᵢ^{*}) and Wⱼ₊₁(Xᵢ^{*}, Yᵢ^{*}) are interpolated,

Wⱼ(γᵢ)=Wⱼ(λⱼ)[Xᵢε[Wⱼ(Xᵢ^{*}, Yᵢ^{*}), . . . , Wⱼ₊₁(Xᵢ^{*}, Yᵢ^{*})]+Wⱼ(γⱼ^{*})]. (Equation IV)

Each interpolated offset, Wⱼ(γᵢ) is then subtracted from each corresponding response Yᵢ between Wⱼ(Xᵢ^{*}, Yᵢ^{*}) and Wⱼ₊₁(Xᵢ^{*}, Yᵢ^{*}) to shift the data to a common baseline. This procedure is performed piecewise for all Wⱼ, j=1, 2, . . . , n/l windows. It is desirable that the window l be larger than the width of any expected amplicon.

FIG. 4 illustrates an exemplary set of spectral data before baseline adjustment is performed and FIG. 6 depicts this same data after adjustment. FIG. 5 depicts source data of a single filter taken during a single scan before adjustment and FIG. 7 illustrates the same data after adjustment. The same procedure is repeated for all of the source data. The spectral intensity data will thereafter have a common baseline of zero amplitude.

It should be noted that it is preferred that baseline adjustment be performed where the photometric data are obtained from fluorophore emissions and the resulting fluorescence recorded by some style of detector. However, performing this algorithm may not be necessary where other methods or techniques of obtaining information from DNA fragments being analyzed are used.

### II. Multicomponenting

If emission spectra from different types of fluorophores overlap, i.e., have some emission wavelengths in common, and are simultaneously recorded by the detector, it may be necessary to remove spectral overlap. This procedure is generally performed after any baseline adjustments are made. To remove spectral overlap, a multicomponent correction preferably is performed on the photometric data. An example of such overlap between the emission spectra from four different fluorophores 76, 78, 80, and 82 is shown in FIG. 8. If desired, removal of spectral overlap can be performed where fluorophore emission occurs at more than one wavelength and this multiple-wavelength emission is detected. This procedure help ensure that the photometric data subsequently analyzed consists primarily of emissions at the wavelength at which a particular fluorophore was intended to emit.

However, in cases where: (1) fluorophore emissions do not have overlapping wavelengths, (2) the fluorophore emits at only a single wavelength, or (3) the detector is capable of selectively detecting emissions at only a single wavelength with no overlap from other fluorophores, it may not be necessary to remove spectral overlap. Additionally, it may not be necessary to remove spectral overlap where the system is not a fluorescence-based detection system. A preferred implementation of a multicomponenting method that is well suited for being implemented in software is presented in more detail below.

In fluorescence-based systems, a suitable detector records excitation emissions by a plurality of different fluorophore types. Such a system is generally referred to as a multicomponent system and is generally capable of simultaneously detecting two or more distinct emission spectra. While each different type of fluorophore is chosen or designed to fluoresce maximally at a specific wavelength, fluorophores typically also fluoresce, to a lesser degree, at some range of wavelengths above and below their wavelength of maximal emission intensity. As a result, fluorophores typically have maximum emission intensity at their designated frequency and a lesser fluorescence intensity at other wavelengths, which can cause the spectrum of wavelengths in fluorophores of one type to overlap with those of another in the manner depicted in FIG. 8.

When detected, these additional emissions lead to a spectral intensity along the overlap that is greater than what it should actually be. This, of course, contributes to variability of intensity values, and it is therefore desirable to compensate for this overlap.

To compensate for spectral overlap, the fluorescence intensity components contributed by fluorophore emissions at wavelengths other than its designated frequency are removed typically by a n-fluorophore multicomponent procedure. In a multicomponent system containing n fluorophores, the fluorescence intensity s, obtained at the i^{th} wavelength, is the sum of the recorded fluorescence intensities at that wavelength from each fluorophore component

$${s}_{i} = {{\sum }^{n}}_{j = 1} {m}_{ij} {f}_{j} \qquad ( Equation V )$$

where the mᵢⱼ are constant coefficients that define the degree of spectral overlap between recorded wavelengths and ƒⱼ is the concentration of the j^{th} fluorophore. Equation V may be represented more compactly in standard matrix form,

\vec{s}=M\vec{ƒ}. (Equation VI)

Here, \vec{s} is a vector with n components, where n is the number of detectors at different wavelengths, and \vec{ƒ} is a vector with m components, where m is the number of fluorophore components. Thus, M is a n×m matrix. In order for M to be inverted, note that n must equal m . For example, in the case of industry-standard 4-fluorophore chemistries, \vec{s} and \vec{ƒ} are vectors each with four elements and M is a 4×4 matrix,

$$[ \begin{matrix}{s}_{1} \\ {s}_{2} \\ {s}_{3} \\ {s}_{4}\end{matrix} ] = [ \begin{matrix}{m}_{11} & {m}_{12} & {m}_{13} & {m}_{14} \\ {m}_{21} & {m}_{22} & {m}_{23} & {m}_{24} \\ {m}_{31} & {m}_{32} & {m}_{33} & {m}_{34} \\ {m}_{41} & {m}_{42} & {m}_{43} & {m}_{44}\end{matrix} ] [ \begin{matrix}{f}_{1} \\ {f}_{2} \\ {f}_{3} \\ {f}_{4}\end{matrix} ] . \qquad ( Equation VII )$$

The matrix coefficients vary depending on the detection instrument and the emission properties of each fluorophore.

Several published methods of determining the matrix coefficients exist. Most are based on an assumption of a linear relationship between the relative spectral intensities contained in the raw data. Once defined, the matrix M provides a transformation to obtain measures of fluorophore concentrations \vec{ƒ} from the actual signals s recorded by the detection apparatus. This process of 4-fluorophore multicomponent transformation uses the specified matrix that, when multiplied against each four-element vector of spectral data, results in a four-element vector of fluorophore concentration

\vec{ƒ}=M⁻¹\vec{s}. (Equation VIII)

The transformation performed by Equation VIII will function correctly only if the signal measurements contained in \vec{s} are approximately linear and correctly positioned to each other relative to a common baseline. Therefore, before multicomponent transformation can be applied, baseline adjustment of the spectral signal, where necessary, must first be performed.

An example of raw fluorophore emissions derived from an ABI 377XL automated sequencer is shown in FIG. 4. In a preferred implementation, using the method of Li and Speed (1999, Electrophoresis, 20, pp. 1433-1442), a 4-fluorophore multicomponent correction matrix was calculated and applied to the previously baselined data. The results of the baseline adjustment and multicomponent correction are shown in FIG. 9.

### III. Noise Reduction

No matter what detection system is used, noise reduction, preferably spectral-based noise reduction, is performed to increase the signal to noise ratio of the photometric data. Such noise reduction is particularly desirable because it significantly reduces the impact of low amplitude background noise and can also reduce the impact of noise spikes. It does so by preferably substantially eliminating the majority of low-amplitude background noise not previously removed by any baselining or multicomponenting procedures that have been performed. Such noise reduction can also be tailored to remove or substantially reduce random noise spikes that sometimes can occur during the detection procedure.

Spectral responses recorded in single- and multi-component systems often contain considerable amounts of additional noise even after other baseline and spectral subtraction corrections have been made. For example, in fluorescent-based systems, a very common type of noise manifests itself as low-amplitude background noise comprised typically of 50 RFI or less.

Another type of noise sometimes observed in fluorescent-based systems manifests itself in the form of a random spike. A random spike typically has an unusually large RFI typically associated with only a single data point. Typically, the RFI of the spike is usually much higher in amplitude than the highest amplitude produced by any spectral emission derived from a real fluorophore present in the system.

It is believed that random noise spikes and other unwanted low-amplitude noise can also contribute to error in other types of detection systems that need not be fluorescence based. As a result of noise reduction, these spikes and other unwanted low-amplitude noise are removed or reduced significantly, thereby smoothing the spectral output of the photometric data and improving its signal-to-noise ratio.

To further enhance accurate measurement of the spectral intensity response, it is desirable to increase the signal-to-noise ratio of the data. This procedure is best performed in a manner designed to minimize the degradation of the true spectral signal while removing the majority of the noise. In its preferred implementation, a highly flexible Fourier-based transform, preferably a discrete Fast-Hartley Transform, is used to perform this noise reduction. A preferred implementation of a preferred noise reduction method is presented in more detail below.

Consider a one-dimensional vector h of real-valued spectral intensities sampled at N uniformly spaced positions (e.g. scans). Denote these samples hₙ where n=0, 1, . . . , N−1. In this implementation, hₙ, corresponds to the amplitude of spectral intensity at scan position n. The output of the Discrete Hartley Transform (DHT) is a real-valued sequence Hₖ given by

$${H}_{k} = \frac{1}{N} {{\sum }^{N - 1}}_{n = 0} {h}_{n} cos ( \frac{2 π nk}{N} ) + sin ( \frac{2 π nk}{N} ) \qquad ( Equation IX )$$

for k=0, 1, . . . , N−1. The resulting spectrum is a mapping of N discrete, time-domain spectral intensity values into N discrete frequency-domain signal components.

These discrete frequency domain signal components take the form of a series, H₀, H₁, H₂, . . . H_{N-1}, where n is equal to the number of scans and k corresponds to the specific frequency. Thus, H₀, is the resultant component that has the lowest frequency, and H_{N-1} is the resultant component that has the highest frequency. The amplitude of each DHT component expresses the relative contribution of that frequency to the periodicity in the original spectral data.

After noise reduction is performed (described below), that data which remains is transformed back into the time domain using an inverse Fourier transform that preferably is an inverse Discrete Hartley Transform (IDHT). The preferred form of the inverse IDHT is given by

$${h}_{n} = {{\sum }^{N - 1}}_{n = 0} {H}_{k} cos ( \frac{2 π nk}{N} ) + sin ( \frac{2 π nk}{N} ) \qquad ( Equation X )$$

for n=0, 1, . . . , N−1 and is used to convert the frequency-domain components of the DHT back into the time-domain values of spectral intensity.

After transforming the data into the frequency domain using equation IX, unwanted high-frequency noise, e.g., high-amplitude noise spikes, is removed by first computing Hₖ for any spectral signal sequence h and then truncating Hₖ at an empirically derived frequency position, Tᵢ. The truncation position, Tᵢ, is chosen to maximize the signal-to-noise enhancement while minimizing any distortions in hₙ.

In the frequency domain provided by the DHT transformation, this preferred form of truncation is equivalent to multiplying a window function g(x) to Hₖ and results in smoothing of undesirably high amplitude spectral intensity data once transformed back into the time domain using the IDHT shown in Equation X. A truncation of this sort using the window function, g(x), is expressed by Equation XI below

Iₖ=Hₖ×g(x) (Equation XI)

where

$$g ( x ) = \{ \begin{matrix}1 {T}_{0} \leq T \leq {T}_{i} \\ 0 otherwise\end{matrix} .$$

Where this type of truncation is performed, Iₖ is substituted for Hₖ in Equation X above.

If it is also desired to remove unwanted low-amplitude noise, e.g., low-amplitude spectral scatter, that was present in the data before being transformed into the frequency domain, a partial truncation may be applied to the amplitude of Hₖ, preferably at or near it lowest frequency component, H₀, as the amplitude of this frequency component contains the vast majority of low-frequency spectral components. Performing partial truncation involves truncating a portion of the amplitude of the frequency component being so truncated.

Both types of truncation can be performed on the same set of data after transformation into the frequency domain. If desired, only frequency component truncation of one or more higher-frequency components can be performed where high amplitude noise reduction is of interest. Similarly, amplitude truncation of at least one lower frequency component can be performed where only low amplitude noise reduction is of interest. In one preferred implementation of a procedure for reducing noise in spectral data, frequency component truncation is performed before amplitude truncation. However, the order in which truncation is performed is not critical.

After truncation is performed, the remaining frequency components are transformed back into the time domain preferably by using the IDHT shown in Equation X. However, after transformation back into the time domain, either type of truncation can give rise to convolution artifacts that visibly manifest themselves as excessive broadening of the spectral intensity distribution in each distinct amplicon.

Multiplication of a Gaussian apodization function, a(x), scaled from (1→0) over the interval (T₀→Tᵢ) in the truncated sequence Iₖ, can be used to effectively attenuate these artifacts before transformation back into the time domain

Jₖ=Iₖ×a(x) (Equation XII)

where

$$a ( x ) = \{ \begin{matrix}{exp}^{- ( \frac{{π}^{2} {x}^{2}}{4 ln 2} )} \\ 0 otherwise \end{matrix} {T}_{0} \leq T \leq {T}_{i} .$$

Where Gaussian apodization is applied, the noise-reduced time-domain spectral signal is recovered by substituting Jₖ for Hₖ in Equation X.

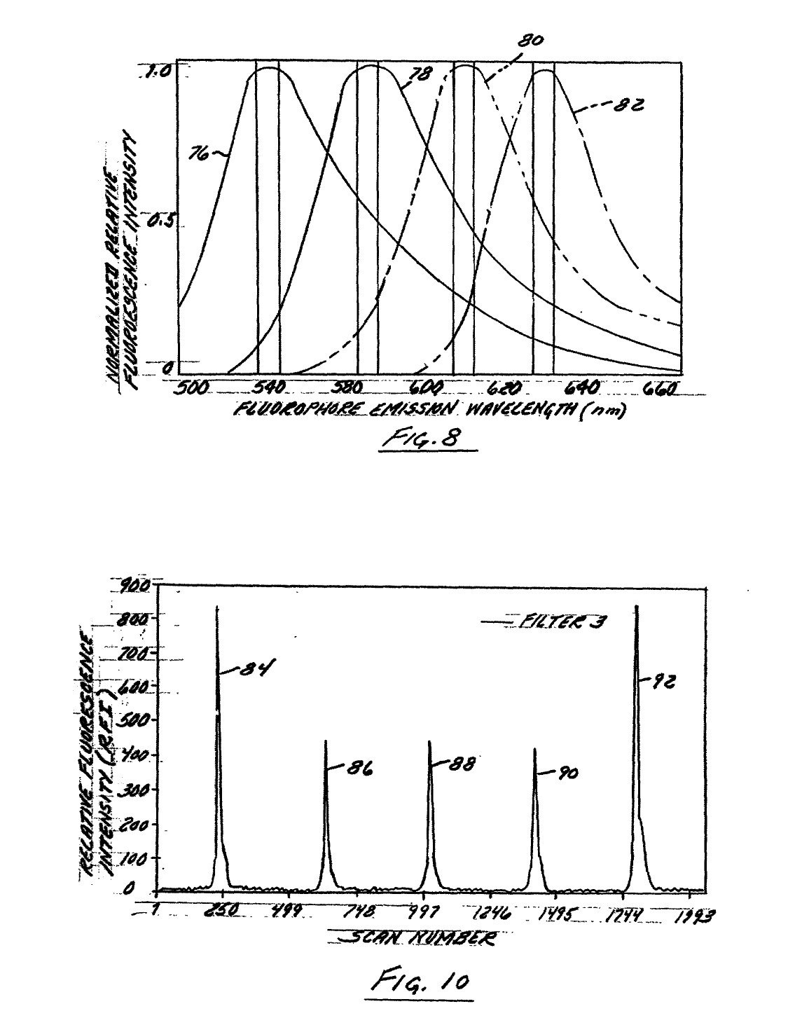

Examples of this preferred noise reduction procedure is graphically depicted in FIGS. 10-17. FIG. 10 illustrates photometric data derived from a one-dimensional source before noise reduction. The X-axis depicts the scan number with the corresponding spectral intensity on the Y-axis. The data is displayed in the time domain with each data point having a value between 0 and 8191. That data has five distinct areas 84, 86, 88, 90, and 92 that exhibit spectral intensities much greater than zero amplitude. These areas contain greater concentrations of fluorophores, which correspond to the location of fluorophore-labeled DNA fragments or groupings of fluorophore-labeled DNA fragments. These locally dense concentrations of fluorophore-labeled DNA fragments are generally termed a “peak” (i.e., a response) of the amplicon. Between each of these peaks 84, 86, 88, 90, and 92, exists an amount of low-amplitude background noise.

In a preferred implementation of a procedure for reducing noise, the photometric data is transformed from the time domain into the frequency domain using a Fourier transform that preferably is a DHT. Equation IX above preferably is used to transform this photometric data from a time domain into a frequency domain, the graphical results of which are shown in FIG. 11.

The transformed photometric data is analyzed after transformation to determine a truncation position, Tᵢ, that preferably will increase the signal-to-noise ratio when the transformed photometric data is retransformed back into the time domain. Where a truncation position, Tᵢ, is determined by visual inspection, graphically displaying the DHT results can help make it easier to determine approximately where the signal-to-noise ratio approaches one. In one preferred implementation, Tᵢ is selected at the frequency where the signal-to-noise ratio is approximately one.

Referring to FIG. 11, the transformed data visibly indicates several lower frequency components having amplitude much different than zero and many higher-frequency components having amplitude very similar to zero. In a preferred implementation, Tᵢ is chosen to truncate at least 20% of the higher frequency components.

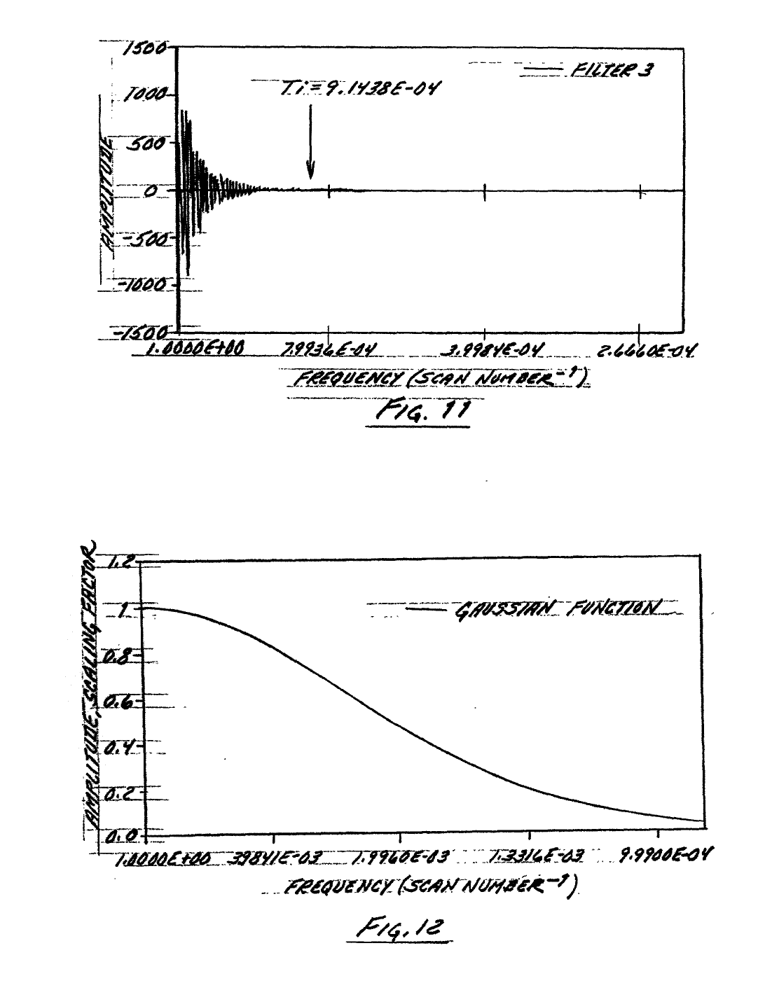

In the preferred implementation graphically depicted in FIG. 11, the truncation position, Tᵢ, is set at a frequency of approximately 9.1438E-04 (scan number⁻¹) as this has been empirically shown to provide good results where the range of each data point is between 0 and 8191. After truncation, the portion of the frequency components having a frequency greater than Tᵢ are simply discarded. Although perhaps 9.1438E-04 is a relatively conservative truncation value, truncation at this position significantly reduces the amplitude of high-amplitude spectral noise. The result of such noise reduction after retransformation back into the time domain is graphically depicted in FIG. 14. The truncation position may differ depending upon the maximum theoretical amplitude of the spectral intensity, which may depend upon the type of detection system used.

If it is desired to reduce low-amplitude spectral noise, one or more of the lower frequency components having amplitude much different from zero can be truncated, preferably by using partial amplitude truncation. In one preferred implementation, H₀, the first frequency component, is truncated such that its amplitude is reduced by at least 25%. Preferably, it is the only frequency component that is partially truncated. In another preferred implementation, each lower frequency component sought to be truncated is multiplied by a truncation coefficient and replaced with its result. For example, in one preferred implementation, H₀ is multiplied by 0.6 and replaced with the result.

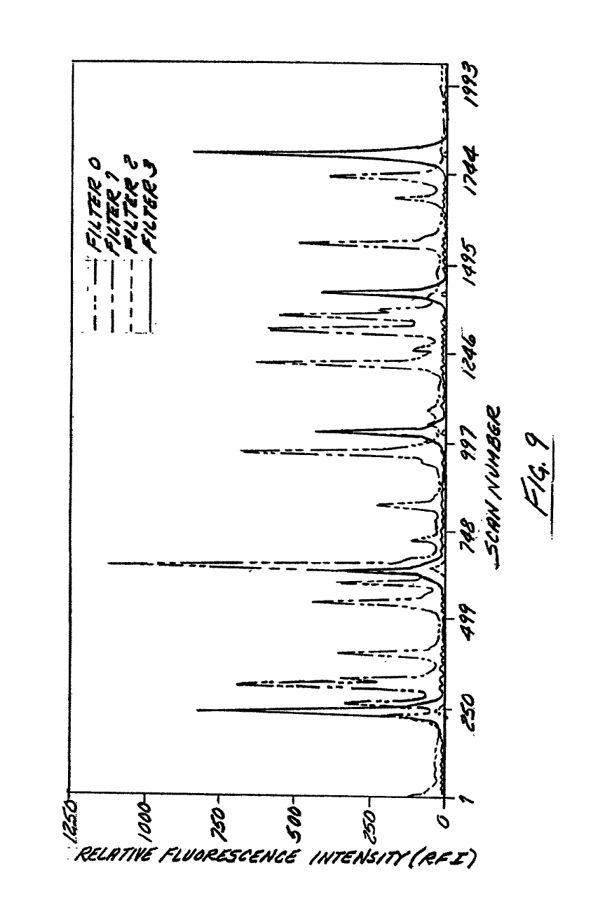

Before transformation back into the time domain, an additional operation can be performed that often provides further enhancement to the noise-reduction procedure. FIG. 12 graphically depicts an apodization finction scaled from 0 to 1 over the range T₀ to Tᵢ. The apodization function is multiplied to the signal amplitudes remaining after any truncation has been performed on the frequency and/or amplitude components. Applying an apodization function to the truncated signal helps attenuate noise that may be introduced as convolution artifacts due to the physical truncation of the signal.

In a preferred implementation, the apodization function is of Gaussian form, which is multiplied to the remaining signal using Equation XII. In FIG. 11, the truncation position, Tᵢ, corresponds to the specific component frequency, 9.1438E-04, at which the original signal was truncated. The apodization function is applied to the signal having a component frequency no greater than that of Tᵢ. In the present example, FIG. 13 illustrates the truncated signal after application of the apodization finction shown in FIG. 12.

Once all manipulation of the frequency-domain components is made, the signal is transformed back into the time domain. In a preferred implementation, Equation X is used to transform the frequency components back into the time domain components of spectral amplitudes. An example of photometric data after noise reduction and retransformation back into the time domain is shown in FIG. 14. As is shown in FIG. 14, the amplitude of each peak has been slightly reduced and low-amplitude background noise between fragment peaks has been dramatically attenuated.

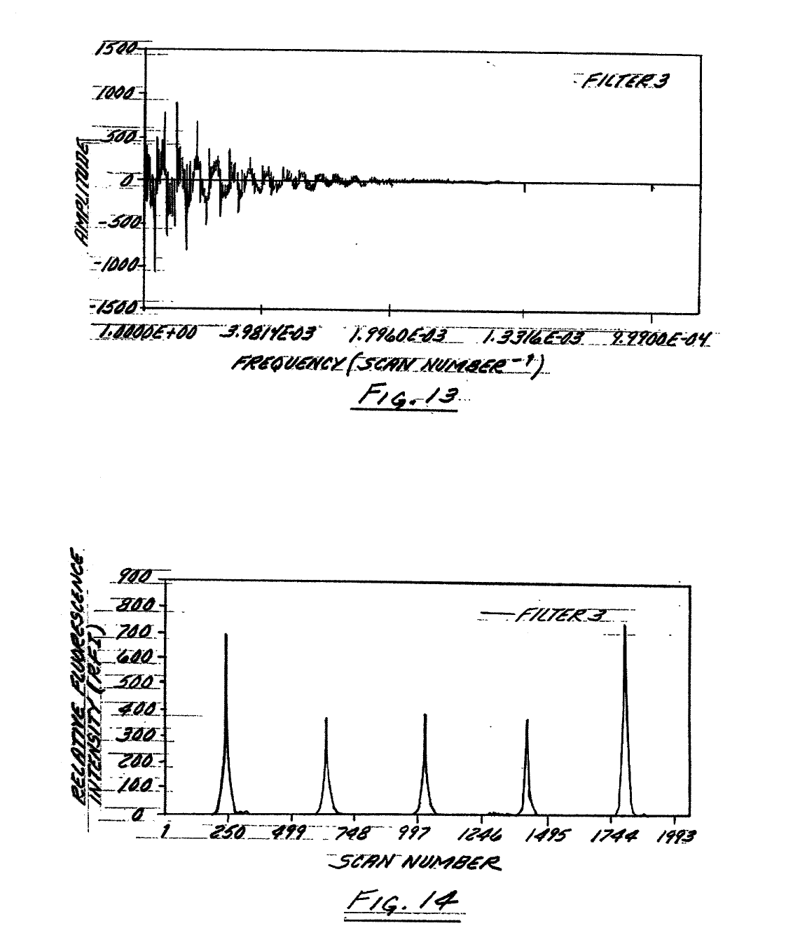

FIGS. 15 and 16 graphically depict the results of high-amplitude noise reduction using high-frequency component truncation. FIG. 15 shows time-domain data of a single baselined and multicomponented noise-spike 94 present at scan number 748. In the same manner previously described, the DHT was applied and the resulting signal was truncated at Tᵢ=9.7656250E-03. The Gaussian apodization function was scaled from 0 to 1 over the range T₀ to Tᵢ and multiplied to the truncated frequency-domain data. Following transformation back into the time domain, the amplitude of the noise spike 94′ is significantly reduced. This is graphically illustrated by FIG. 16, which shows the spike 94 of FIG. 15 after transformation using DHT, performing high-amplitude truncation, applying a Gaussian apodization function, and transforming the data back into the time domain using IDHT. As a result of this high-amplitude noise reduction, the impact of this noise spike 94′ on the subsequent analysis discussed below preferably is minimal, if not completely negated.

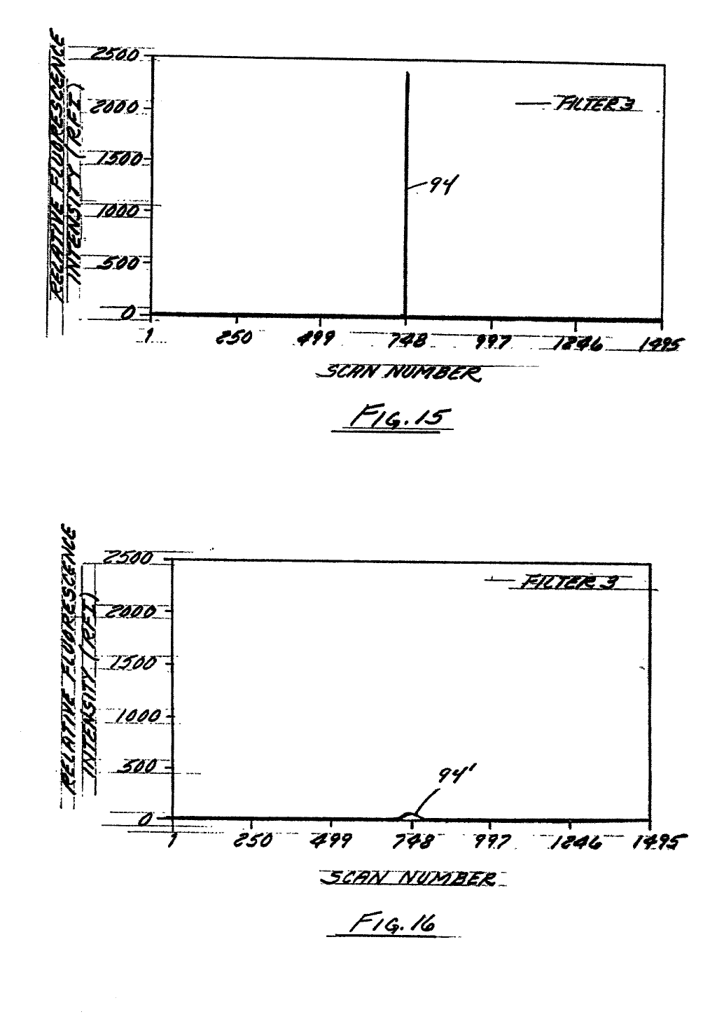

This same noise reduction procedure may be applied to two-dimensional source data, e.g., a gelfile. FIG. 17A graphically illustrates the raw spectral data from a portion of a gelfile. FIG. 17B graphically illustrates the results of this noise reduction procedure applied to this same portion of the gelfile.

An advantage of performing noise reduction in this manner is that it later aids in amplicon analysis and that it is well suited for implementation in software. For example, locating the top or apex of each amplicon is made easier because the noise between amplicons is greatly reduced. The reduction in noise also makes it easier to detect the leading and trailing edges of each amplicon, thereby making it easier to more accurately estimate parameters of each amplicon, such as maximum spectral amplitude, length or width. Moreover, reducing high-amplitude noise spikes aids greatly in achieving a suitable range of spectral intensities in which to rescale the data. Taken together, all of these features greatly enhance the accurate estimation of total spectral intensity discussed herein.

### Rescaling of Data

In one preferred implementation, raw and/or processed photometric data are rescaled preferably to facilitate visual inspection of the data. Depending on the format in which the photometric data is initially presented, or what procedures are to be applied to the data, e.g. baseline, multicomponent, noise-reduction, etc., resealing may or may not be desirable or necessary. If rescaling is performed, photometric data are resealed into another digital representation that can be preferably more easily manipulated and used.

In one preferred implementation, photometric data are rescaled into a different digital format after applying baseline, multicomponent, and noise reduction procedures. For example, where an ABI gelfile is rescaled, two-byte (INT[2]) photometric data having equivalent decimal values ranging from 0 to 8191, i.e., 2¹³ values, is converted into two-byte (INT[2]) photometric data having equivalent decimal values ranging from 0 to 255, i.e., 2⁸ values.

Where resealing is performed, the rescaling of the photometric data into a more common and compact form is advantageous as it permits the photometric data to be more easily displayed and analyzed in visual manner. Additionally, rescaling into a more common form means that the photometric data can be used in any one of dozens of commercially available programs capable of accepting standardized image data.

It should be noted that rescaling could be performed wherever the photometric data comprises fluorescent data. However, performing this step may not be necessary where other methods or techniques of obtaining information from DNA fragments being analyzed are used.

In one preferred method of resealing, a histogram of spectral intensity values is first made of the photometric data. Thereafter, the data are scaled preferably to an industry-standard 8-bit scale such that the magnitude of the photometric data ranges from 0 to 255. To make the data easier to rescale, a portion of the largest and smallest histogram values can be removed. For example, in one preferred implementation, the largest and smallest three percent of the spectral amplitudes in the photometric data are first removed, preferably by truncation or magnitude reduction, to facilitate resealing the data into an 8-bit format.

Where the data is rescaled, it preferably is reformatted into a standard graphics format, such as bitmap (BMP), Portable Network Graphics (PNG), Portable Pix Map (PPM), Portable Gray Map (PGM), Tagged Image File Format (TIFF), Graphics Interchange Format (GIF), PC paintbrush (PCX), Joint Photographic Experts Group (JPEG), Flashpix (FPX), or another computer readable format. For example, in one preferred implementation, the rescaled photometric data is written to a Portable Gray Map (PGM) file format. In doing so, a header is added to the photometric data that denotes it as being in particular graphics data format and that includes certain dimensions of the file or other necessary information. When displayed on a screen, such as a computer driven display or the like (see below), a graphical image of the recorded spectral intensities can be depicted, such as shown in FIGS. 17A and 17B.

### Display of Data

It is often desired to have the ability to display the photometric data at some point. For example, it is desirable to display the photometric data in order to inspect the results of the marker assay. Inspecting the results also provides a means to evaluate the progress of an experiment and also enables lane tracking to be performed, especially where two-dimensional photometric data is involved.

The majority of genetic marker-assay detection systems record the presence of a substance (response) as an ordinal response preferably on a uniformly defined time scale. Typically, these responses are measured as spectral intensities, indicating relative concentration, ranging from 0, i.e., no response, increasing to an upper value that typically is limited by the physical properties of the detector. These values may be translated into graphical representations suitable for human visualization.

Consider each response recorded by the detector as a single element of amplitude in a vector of integer-valued measurements ordered by time. Each element may then be defined as a single graphical pixel in a one- or two-dimensional array. These pixels may be displayed graphically by constructing a grayscale or false-color map that allows for visual discrimination among the spectral response data.

Due to the fact that commonly available graphical formats cannot directly display unprocessed photometric data, the data can be transformed into an appropriate form by first resealing it as described previously. For example, in the case of industry-standard four-fluorophore chemistries, after rescaling, one preferred implementation maps the four-component fluorophore intensity space into a three-component Red-Green-Blue (RGB) color space. This is accomplished by assigning four different RGB colors to each of the four fluorophores—red, green, blue, and yellow. Individual component spaces are defined by mixing the four colors according to the intensity of the corresponding pixel at each position in the pixel vector.

### IV. Lane Tracking

Where slab-gel electrophoresis is performed, multiple samples are typically loaded across one dimension of the gel (X) and simultaneously separated along the other dimension (Y). Compared to capillary electrophoresis, slab-gel electrophoresis devices do not have any intrinsic capability to precisely delimit individual samples on the gel itself. For example, standardized square-shaped loading combs provide an organization of sample wells that delimit, preferably passively, each sample with a small blank area, but only offer a simple visual means of distinguishing among adjacent sample lanes. This feature is insufficient for precisely defining DNA fragments (e.g. amplicons) present in individual sample lanes. Therefore, each sample lane, and therefore amplicons contained within that lane, must be defined by a human operator or by a computer implemented algorithm designed to perform lane tracking 50 (FIG. 1). This procedure is also necessary where a “sharks-tooth comb” is used, which results in almost no visually ascertainable gap between sample lanes.

In a present implementation, the task of defining the individual amplicons belonging to each sample lane is accomplished by manually constructing a tracking-spline that bisects each amplicon present in the marker assay for each sample lane. Spectral intensities are averaged laterally over a region of the sample lane, e.g., a region defined by the average width of a sample lane (cf. channel). The result of this procedure is a one-dimensional vector of averaged spectral intensities spaced over uniformly spaced time intervals (cf. sample file).

In another preferred implementation, where lane tracking is already performed and saved to a file, the tracking information is read directly from the file and later used to obtain the location of the lanes. For example, where the source data is an ABI-generated gelfile processed with ABI Genescan Analysis or Sequencing Analysis software, the lane tracking information can be extracted from the gelfile and subsequently used. In one preferred implementation, the lane tracking information is extracted and stored for later use, such as by saving it in a file. The location of each lane is obtained from the stored lane tracking information and used with the processed photometric data to show the location of the lanes when the photometric data is displayed. For example, processed and rescaled photometric data can be displayed and the lanes can be superimposed on the photometric data using the extracted lane tracking information.

Once defined, the lane-tracking data will serve as input data to enable extraction of a one-dimensional vector of photometric data from the gelfile (cf. sample file). This procedure is implemented in one of two preferred methods: (1) for each time interval or scan, the recorded value of spectral intensity (cf. channel) nearest to the tracking spline is extracted or (2) for each time interval (scan), an average value of recorded spectral intensity in a defined proximity to the tracking spline is extracted. In one preferred implementation, the proximity is defined as the number of channels defining the average width of an amplicon in the gel, typically 4 or 5 channels on an ABI 377XL automated sequencer.

Once extracted, the photometric data will be used to: (1) identify each amplicon in a sample, preferably including molecular weight standards, (2) estimate amplicon parameters such as maximum spectral intensity (amplitude), width and position, and (3) generate a sizing curve based on data derived from included molecular-weight standards. Information gathered from each procedure allows the assignment of a molecular size, either as a molecular weight or molecular mobility, to amplicons of previously unknown size.

### V. Amplicon Identification

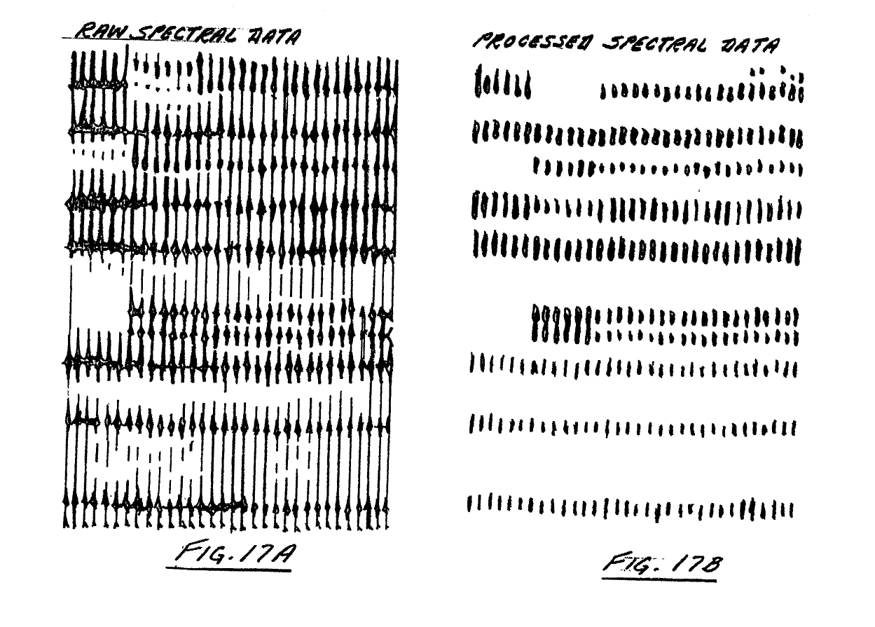

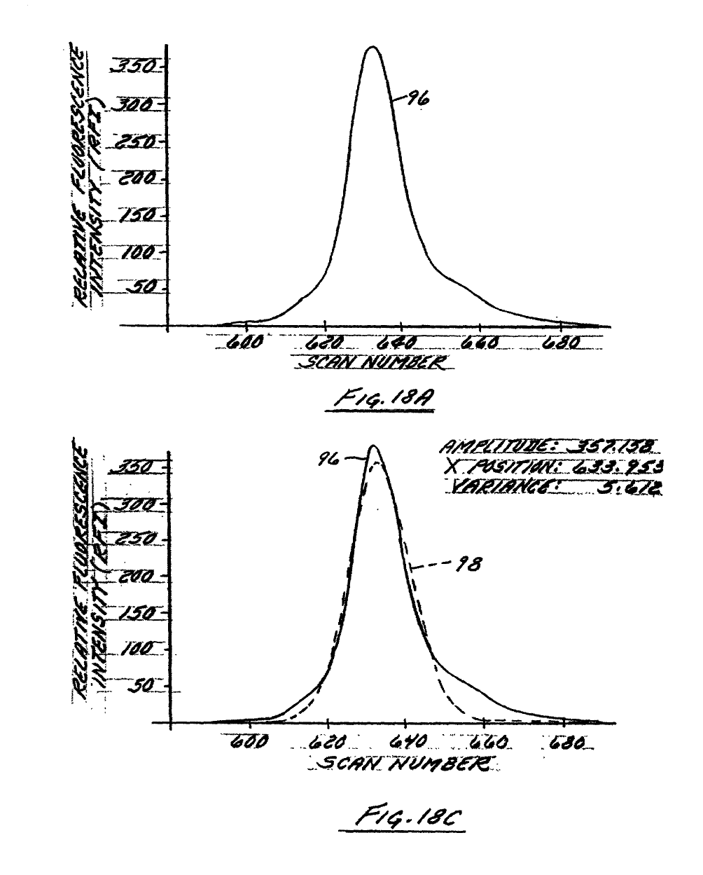

The photometric data shown in FIG. 14 depict five points of maximally increased spectral intensity, i.e. a spectral peak, surrounded on either side by regions of reduced spectral intensity. The purpose behind identification of these areas 52 (FIG. 1) is that they indicate the presence of locally-dense concentrations of fluorophore-labeled DNA fragments. Taken together, these regions define, in FIG. 14 for example, five differently-sized amplicons. The location of the spectral peak relative to its position on the X-axis (i.e. scan number) within any single amplicon is defined to be the absolute center of that amplicon. The distinction of the peak maximum is important because even if it is assumed that all DNA fragments comprising a single amplicon are identically-sized, diffusion of these fragments does occur. Therefore, instead of occupying a single discrete location in the gel, the DNA fragments comprising an amplicon are actually spread out over a region and confound the “true” position of the peak. An example of the spectral distribution of a single amplicon, in one dimension, is shown in FIG. 18A. Each amplicon has associated with it a maximum height or amplitude (peak) with an inflection point or apex, and a width or variance. In the case of fAFLP, as is exemplified here, these localized regions are indicative of an amplicon, provided that the peak maximum is greater than a predefined threshold.

In a preferred implementation of a procedure for locating amplicons, values of spectral intensity are sequentially examined and compared to spectral intensities immediately preceding and following the current location. At the leading edge of a putative amplicon, i.e., where spectral intensity begins to increase steadily, each successive value of spectral intensity increases until a maximum value is reached before the spectral intensity begins to decrease. At the position (scan number) where spectral intensity first decreases, the scan number immediately prior to that position is identified as being the location of maximum spectral intensity (i.e. the inflection point or peak apex) of that putative amplicon. An advantage of this preferred implementation is that it is capable of not only quickly and simply identifying the apex of any amplicon, but it also is capable of identifying the majority of peak apexes should adjacent amplicons overlap.

This procedure also determines a putative position, typically as a scan number, of each amplicon relative to all other amplicons present in a given sample. The search algorithm also compares the value of spectral intensity at each putative position of a putative peak apex against a predetermined threshold value of spectral intensity. When the threshold is exceeded, the position associated therewith is noted and the spectral peak of the amplicon is identified. Those putative positions that do not equal or exceed the threshold are discarded.

After the peak apex has been identified, such as in the manner discussed immediately above, the trailing edge of the peak can be identified. Knowing the location of the trailing edge of an amplicon and the location of its leading edge, the amplicon full-width at half-maximum amplitude, i.e., amplicon variance, may be estimated. More specifically, in one preferred implementation, the amplicon variance preferably is calculated as the distance between the leading and trailing edges of an amplicon at the point of one-half the distance to the peak apex.

However, in a preferred implementation of a method of this invention, only the leading edge and the peak apex need be determined to provide suitable information for determination of amplicon variance. In another preferred implementation, only the trailing edge and peak apex need be determined. Preferred implementations of a procedure for locating an amplicon, its peak apex, and its variance, are discussed below.

The presence of all amplicons or DNA fragments (e.g. DNA standards) in each marker assay must first be ascertained. This is accomplished by scanning the data vector defined by the tracking-spline for amplicons containing spectral intensities greater than a defined minimum-response value C. Let ƒ be a one-dimensional vector of spectral intensities arranged by scan number. Assume that ƒ is continuous and differentiable on the interval (a, b) defined to be the beginning and end, respectively, of ƒ. Define ƒ to be increasing in (a, b) if

$$\frac{ⅆ f ( x )}{ⅆ x} > 0 x ε ( a , b ) \qquad ( Equation XIII )$$

and decreasing in (a, b) if

$$\frac{ⅆ f ( x )}{ⅆ x} < 0 x ε ( a , b ) \qquad ( Equation XIV )$$

Suppose that ƒ contains possibly many values cᵢ, such that

$$\begin{matrix}\frac{ⅆ f ( {c}_{i} )}{ⅆ c} = 0 \\ If & ( Equation XV ) \\ \frac{ⅆ f ( x )}{ⅆ x} > 0 x ε ( a , {c}_{i} ) \\ and & ( Equation XVI ) \\ \frac{ⅆ f ( x )}{ⅆ x} < 0 x ε ( {c}_{i} , b ) & ( Equation XVII )\end{matrix}$$

then ƒ has a local maximum at x=cᵢ. Provided that cᵢ≧C, each cᵢ defines a single spectral peak with an apex located at the corresponding position xᵢ. As a result of knowing the peak apex position, its maximum amplitude can also be ascertained.

Referring to FIG. 18, FIG. 18B illustrates a portion of the leading edge of a portion of the amplicon shown in FIG. 18A beginning at scan numbers 623 and 635, respectively. Although the peak apex is not shown in FIG. 18B, the vertical line centered at position X_{LM} corresponds to the location of the peak apex, i.e., the position of the peak maximum amplitude, with magnitude Y_{LM}. The solid circles indicate observed values of spectral intensity at their corresponding scan numbers, X_{(L)} and X_{(L+1)}. The open circle shows the intersection of an imaginary dashed line drawn perpendicular to the peak apex position centered at one-half the maximum amplitude of the peak apex, Y_{(LM/2)}, located at position X_{α}.

To obtain an estimate of the amplicon variance (full-width at half-maximum amplitude), first define the quantity Y_{LM} as the local maximum amplitude at position cᵢ=X_{LM} of the amplicon. An approximation of the position X_{α} where a line originating at (X_{LM}, Y_{LM}/2) perpendicular to (X_{LM}, Y_{LM}) crosses the leading edge of the amplicon is given by:

$${\hat{X}}_{α} = {X}_{( L + 1 )} - ( \frac{( {Y}_{{X}_{( L + 1 )}} - ( {Y}_{LM} / 2 ) )}{( {Y}_{{X}_{( L + 1 )}} - {Y}_{{X}_{L}} )} ) \qquad ( EquationVIII )$$

where Y_{X}_{_{L}}≦(Y_{LM}/2)≦Y_{X}_{_{(L+1)}} is constrained by the condition (X_{L+1}−X_{L})=1. Assuming roughly symmetrical amplicon proportions, a good estimate of the amplicon full-width at half-maximum amplitude (FWHM) is given by: [symbol] =2×(X_{LM}−X_{α}). (Equation XIX)

The calculated estimates of peak apex position, X_{LM}, maximum amplitude, Y_{LM}, amplicon full-width at half-maximum amplitude, [symbol] , provide starting values required by the nonlinear amplicon-parameterization procedure described below.

A preferred implementation of a procedure for detecting a leading edge of an amplicon involves scanning through the data vector and determining the presence of a plurality of consecutively increasing spectral intensities. Where the presence of a putative amplicon is identified, it is accepted as an amplicon if the peak apex is above a threshold spectral intensity value. Otherwise, it is rejected. Such a preferred implementation helps minimizes the occurrence of false negatives and false positives. In cases where this procedure is implemented in software, a message preferably is generated.

In one preferred implementation, a leading edge is confirmed when five spectral intensities are observed to consecutively increase. An amplicon is accepted if the maximum spectral intensity of the series of increasing spectral intensities detected is greater than a peak spectral intensity threshold, for example, of 50 RFI. If desired, a greater number of consecutively increasing spectral intensities can be used to detect the presence of a leading edge where the risk of false positives is desired to be very small. If desired, the threshold spectral intensity value can be higher or lower depending on the needs of the user.

In another preferred implementation, a check is made two determine whether the spectral peaks of two amplicons are located relatively close together. For example, where two spectral peaks are detected within 10 datapoints or scans of each other, the location (scan number) of each peak is identified and a message preferably is generated. Identifying incidences of two closely spaced spectral peaks enables a more detailed manual examination or automated analysis of this region to be made as such an occurrence is indicative of amplicon overlap. A second check preferably is also performed to determine the presence of “flat maxima”, that is, a region where spectral intensities are the same as the maximum spectral amplitude that the detector is capable of detecting or outputting. A message preferably is also generated where a flat maxima occurs so that this region of data can be more closely examined and disregarded, if necessary, as these values are prone to extreme amounts of error.

### Estimation of Amplicon Parameters Using Orthogonal Distance Regression

Despite having attenuated significant amounts of noise and possibly having performed other procedures to enhance the quality of the photometric data, there may still be appreciable amounts of error in the location, amplitude, and width of a DNA fragment or amplicon. For instance, slight variations in the polyacrylamide gel matrix may cause identically-sized DNA fragments or amplicons to travel through the gel at different rates. This can cause location error, which can ultimately lead to error in assigning a molecular size, either, e.g., as a molecular weight or molecular mobility, even when the DNA fragment or amplicon is compared to a known standard.

Other types of error may also exist. For example, error in spectral intensity measurement manifesting itself as amplitude error may lead to error in amplicon variance, i.e. full-width at half-maximum amplitude. Fragment location error can also occur because the detector has limits to the amount of data it can record. As a result, for example, the apex of an amplicon can be completely missed by the detector, causing its location to be erroneously estimated. Other limitations in the detection process, including scanning error and frequency, can also result in incomplete signal capture and lead to such errors.

To help correct such error, it is desirable to fit an idealized function or curve to each amplicon identified in the source data. For example, where the system is a fluorescence-based system, the data comprises photometric data representing spectral intensities. The fitted function essentially replaces or supplants each amplicon defined by the observed data. This helps recapture and/or restore lost signal and can help correct location error.

In one preferred implementation of an algorithm for providing additional error correction, it is assumed that the distribution of spectral intensity in each identified amplicon can be approximated as being Gaussian in shape, and a Gaussian function is therefore fitted to each amplicon. Where the apex of an identified amplicon has somehow been missed or skewed, fitting such an ideal and continuous curve to data defining the amplicon can recover lost signal at the apex, while also helping to ascertain more accurately its true location. In fact, depending upon the resolution of the detector, the fitting of a continuous curve or function to an identified amplicon that is defined by discrete digital actual data can help more accurately define how the amplicon should have ideally been detected. The result is a continuous curve or function that adjusts itself to the observed data in a manner that more accurately reflects its expected signal amplitude value at each location of the amplicon.

In one preferred implementation, estimates of apex amplitude, apex position, and variance of an amplicon derived from the implementation of the amplicon response identification procedure disclosed in the preceding section, are used as starting parameters for the Gaussian-function fitting procedure. Preferably, a nonlinear, orthogonal distance regression (nODR) fitting algorithm is used. Such an algorithm assumes that there is error not only in the observed value of spectral intensity (i.e. the response variable) but also the position of the response (i.e. the predictor variable). This methodology differs markedly compared to traditional regression procedures which assume no error in the predictor variable. Since it as an objective of the invention to more accurately and precisely define the location of an amplicon, the use of nODR is clearly advantageous. In a preferred implementation, the location of the spectral peak corresponds to a scan number and is defined as the X variable that is also referred to as being the predictor variable. The amplitude at the apex position corresponds to spectral intensity and is defined as the Y variable that is also referred to as being the response variable.

Such a nonlinear fitting algorithm helps correct for or compensate for peak apex location, amplicon variance, and spectral amplitude errors. In one preferred implementation, a suitable nonlinear least-squares orthogonal distance regression approach, such as that set forth in Equations XX to XXIV below, preferably is implemented using ODRPACK. ODRPACK is a configurable subroutine library that is publicly available from the repository at NetLib at www.netlib.org. A preferred implementation of an algorithm that fits a Gaussian curve to each peak using orthogonal distance regression is discussed in more detail below.

All amplicons derived from the marker assay must be assigned a unique value so that homology, e.g., as defined by its size, may be assigned unambiguously across multiple sample assays. This is accomplished by defining a relationship between the molecular weight and/or molecular mobility of a DNA fragment (or an amplicon) in a gel and its size. Size-standardized DNA fragments included in each lane with the products of the marker assay form the basis by which size-unknown amplicons are estimated.

Let (Xᵢ, Yᵢ), i=1, 2, . . . , n be a one-dimensional vector of observed spectral intensity data with true, i.e. unknown, values (xᵢ, yᵢ), i=1, 2, . . . , n. Assume that δᵢ and εᵢ is the random error associated with (xᵢ, yᵢ) is

Xᵢ=xᵢ−δᵢ

Yᵢ=yᵢ−εᵢ (Equation XXI)

Assume that the values of the spectral intensity response yᵢ are a nonlinear function of the position predictors (e.g. scan numbers) xᵢ and a set of unknown parameters β. Then the observed value of yᵢ satisfies yᵢ=ƒ(xᵢ; β) or

Yᵢ=ƒ(Xᵢ+δᵢ; β)−εᵢi=1, 2, . . . ,n (Equation XXI)

for some actual but unknown values, β*, β*=(β₁, β₂, . . . , βₚ) where p is the number of parameters to be solved. The explicit orthogonal distance regression (ODR) procedure approximates β* by defining an orthogonal distance rᵢ from the point (Xᵢ, Yᵢ) to the curve ƒ(\tilde{x}, \tilde{β}) where indicates an unspecified value of the specific variable. Given that the errors are normally distributed and have zero-mean, i.e. δᵢ˜N(0, σ_{δ}_{ᵢ}²) and εᵢ˜N(0, σ_{ε}_{ᵢ}²), the minimization of the sum of squares of rᵢ²=[\tilde{δ}ᵢ²+\tilde{ε}ᵢ²] is the maximum likelihood estimate of β*. Thus, β* is the solution of

$${min}_{{β}^{*} , \tilde{δ} , \tilde{ϵ}} {\sum }_{i = 1}^{n} [ {\tilde{δ}}_{i}^{2} + {\tilde{ϵ}}_{i}^{2} ] \qquad ( Equation XXII )$$

subject to the constraint

Yᵢ=ƒ(X_{i+\tilde{δ}}ᵢ; β*)−\tilde{ε}ᵢi=1, 2, . . . , n. (Equation XXIII)

Since εᵢ in Equation XXIII is linear, εᵢ can be eliminated thereby resulting in an unconstrained minimization problem

$${min}_{{β}^{*} , \tilde{δ}} {\sum }_{i = 1}^{n} ( {[ f ( {X}_{i} + {\tilde{δ}}_{i} ; {β}^{*} ) - {Y}_{i} ]}^{2} + {\tilde{δ}}_{i}^{2} ) . \qquad ( Equation XXIV )$$

In a preferred implementation, it is assumed that the distribution of fluorophore intensity in each fully resolvable amplicon is closely approximated by a Gaussian function

$${Y}_{i} \approx f ( {X}_{i} ; β ) ≡ {β}_{1} \times exp [ - ( \frac{{( {X}_{i} - {β}_{2} )}^{2}}{2 {β}_{3}^{2}} ) ] \qquad ( Equation XXV )$$

where β₁ describes the observed amplitude at the peak apex, β₂ is a location parameter corresponding to the peak apex position, and β₃ describes the amplicon variance that preferably is full-width at half-maximum apex amplitude. For multiple amplicons in the same data vector, Equation XXV may be summed and solved simultaneously over all p amplicons of interest,

$${Y}_{i} \approx f ( {X}_{i} ; β ) ≡ {\sum }_{β = 1}^{{3}_{p}} {β}_{1} \times exp [ - ( \frac{{( {X}_{i} - {β}_{2} )}^{2}}{2 {β}_{3}^{2}} ) ] + … + {β}_{3 p - 2} \times exp [ - ( \frac{{( {X}_{i} - {β}_{3 p - 1} )}^{2}}{2 {β}_{3 p}^{2}} ) ] . \qquad ( Equation XXVI )$$

Estimates of the required starting values for each of the 3p parameters in Equation XXVI, i.e., β₁, β₂, . . . , β₃ₚ, are obtained by preferably first fitting Equations XXV or XXVI to the data using an ordinary nonlinear least-squares (nOLS) procedure (i.e. assuming no error is observed in the predictor variable). Estimates of starting values for each of the 3p parameters required for this fit preferably are provided by the amplicon parameter estimates derived preferably from the initial amplicon identification step described above. Upon convergence of the nOLS algorithm, such estimates of β are then used as starting values for fitting Equations XXV or XXVI to the data using a nODR procedure (i.e. assuming the predictor variable contains error). By first estimating the 3p parameters with the nOLS procedure, the amount of time necessary for convergence of the nODR algorithm is reduced significantly. The estimates of β from the nODR procedure are also used to preferably generate a molecular sizing curve in a manner such as described below.

The above methodology can also preferably be implemented for the two-dimensional case except that two additional parameters are required. The first describes amplicon position in the second dimension and the other describes amplicon variance in the second dimension. Where such is desired, estimates of peak apex height, β₁, peak position in the first dimension (e.g. X), β₂, amplicon variance in the first dimension (e.g. X), β₃, peak position in the second dimension (e.g. Y), β₄, and amplicon variance in the second dimension (e.g. Y), β₅, can be obtained using a version of Equation XXV or XXVI modified for the two-dimensional case. For example, Equation XXV modified for the two-dimensional case becomes

$${β}_{1} \times exp [ - ( \frac{{( {X}_{i} - {β}_{2} )}^{2}}{2 {β}_{3}^{2}} ) ] \times exp [ - ( \frac{{( {Y}_{i} - {β}_{4} )}^{2}}{2 {β}_{5}^{2}} ) ] . \qquad ( Equation XXVII )$$

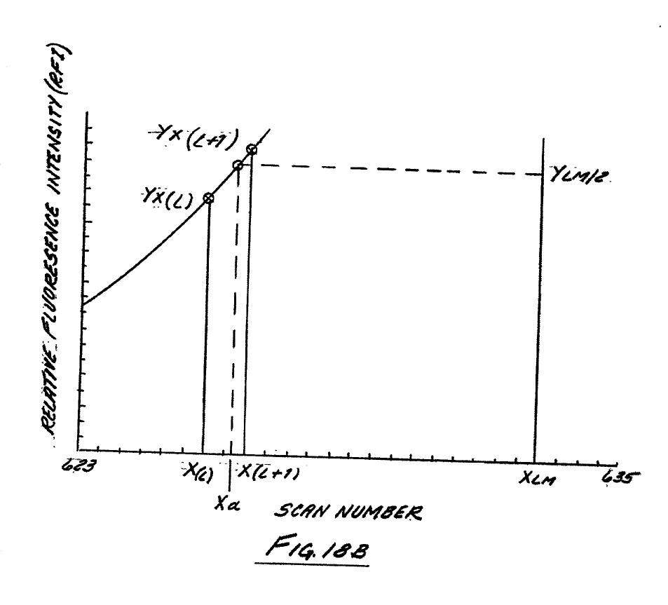

An example depicting a preferred one-dimensional implementation is graphically represented in FIGS. 18-19. FIG. 18A illustrates a baselined, multicomponented, and noise-reduced spectral peak 96. FIG. 18B illustrates a portion of the amplicon used to determine a preferred variance, namely, the full-width at half-maximum. FIG. 18C illustrates an nODR fit of a Gaussian function 98 (in phantom) overlaid on top of the amplicon shown in FIG. 18A. Also displayed in FIG. 18C are the estimates of the three fitted parameters of peak amplitude, “AMPLITUDE,” position, “X POSITION,” and amplicon full-width at half-maximum amplitude, “VARIANCE.” For subsequent analyses, the fitted Gaussian amplicon 98 may or may not replace the original amplicon 96, depending on the needs of the user. For example, when constructing an amplicon sizing curve, in replacing the original peak 96, the fitted values of Yᵢ generated using Equations XXV or XXVI as a function of Xᵢ preferably replace the observed (Xᵢ, Yᵢ) data values.

In another preferred implementation, parameters of multiple amplicons may be simultaneously estimated using a sum of multiple Gaussian functions, preferably using Equation XXVI. FIG. 19A illustrates five baselined, multicomponented, and noise-reduced amplicons 100, 102, 104, 106 and 108. FIG. 19B illustrates a nODR fit of five Gaussian functions 110, 112, 114, 116 and 118 (in phantom) overlaid on top of the amplicons shown FIG. 19A. Also displayed in FIG. 19B are the estimates of the fitted parameters of peak apex amplitude, position and variance for each amplicon.

In an alternately preferred implementation, a starting value for amplicon variance (e.g. with reference to Equation XXV, parameter β₃) is provided from a table of estimated variances based on peaks observed in a DNA molecular-weight standard. The table includes the computed variance for each standard used. For example, where 22 standards are used such as with DNA standard ILS-600, the table contains a computed variance for each DNA standard. As such, the approximate location of the amplicon being fitted can be used to select an estimated variance using the DNA standard closest to that location. In a preferred implementation, linear regression is used to obtain a variance estimate where the peak apex location of the amplicon being fitted lies between two of the DNA standards. Thereafter, this amplicon variance estimate used as a starting value at the initiation of either minimization routine. In the preferred implementation set forth above, this variance estimate is input as variable β₃ into, for example, Equation XXV above.

Although the use of an estimated variance value that is based upon a standard rather than an actual amplicon may comprise an estimate that is not exact (especially as the variance is dependent on the amount of DNA deposited onto the gel), it appears accurate enough to allow the nOLS minimization routine to more quickly converge to an accurate estimate of variance than by simply guessing. This is due to the fact that estimates of amplicon peak apex location and amplitude are generally much more accurate, thereby reducing the necessity of an extremely accurate starting value of estimated amplicon variance. The amplicon variance estimate obtained by using estimates obtained from DNA standards is simply a reasonable estimate that helps minimize computational time of the minimization routine implemented in software, while still ensuring convergence to an accurate estimate of variance for the amplicon being fitted.

In another preferred implementation, estimates of amplicon variance used for starting values in the nOLS and/or nODR least-squares minimization routines are computed directly from the full-width at half-maximum amplitude value in the amplicon response identification procedure discussed in the preceding section. Preferably, the estimate of amplicon variance used as a starting value in the nODR least-squares minimization routine is the full-width at half-maximum amplitude value obtained from the initial nOLS fit or the amplicon peak identification procedure.

In another preferred implementation, the nODR procedure is used to estimate amplicon parameters of position, variance, and amplitude for each amplicon individually. Starting values for amplicon peak amplitude, position, and variance for the minimization procedure are obtained from the amplicon response procedure described above. Once all fitted parameters for each individual amplicon is obtained, these values can be used for starting values in the minimization procedure where parameters of all amplicons taken together are estimated.

This preferred fitting procedure advantageously helps minimize variability when binning amplicons based on their estimated sizes. This increases not only the certainty of ascertaining an accurate bin for each amplicon, but does so by increasing the accuracy of the molecular size obtained. For example, as a result of using this preferred fitting algorithm, molecular sizes of fragments associated with each amplicon can typically be estimated to a precision of at least 0.333 nucleotide bases as discussed in more detail below.

### VI. Generation of Amplicon Sizing Function

After nonlinear orthogonal distance regression (nODR) has been performed to obtain the best-fit parameters of peak amplitude, location, and variance for each amplicon, a sizing function or curve preferably is generated with DNA standards of known size. This preferably is done using DNA size standards that are electorphoresed simultaneously with amplicons from each sample. The function or curve is used to estimate the size of size-unknown amplicons as either, but not limited to, (1) a molecular weight, e.g. in units of nucleotide bases or (2) a molecular mobility, e.g. in units of scan numbers. By using molecular weight or mobility data from DNA size standards electrophoresed concurrently with size-unknown sample amplicons, the molecular weights or mobilities of such amplicons may be estimated 54 (FIG. 1).

In a preferred implementation that uses molecular weights to estimate the sizes of amplicons, 22 DNA fragment standards ranging in size from 60 to 600 nucleotide bases, such as with ILS-600, comprise the DNA-standard sample. Prior research has demonstrated that an approximately linear relationship exists between the molecular weight and the distance traveled (mobility) of single-stranded DNA fragments in a gel matrix. This approximate linear relationship has been used in the past in the form of a sizing-curve that is in turn used to extrapolate a molecular weight for any amplicon or DNA fragment of unknown size, within the size limits of the DNA standard used.

However, while this assumed linear relationship may indeed apply to DNA fragments having molecular weights within the range of approximately 100 to 400 nucleotide bases in length, it is believed that this assumption does not apply to DNA fragments having very large or very small molecular weights. As a result, estimates of molecular weights of DNA fragments outside of this range that are made assuming that such a linear relationship exists can be erroneous. Moreover, recent experimental evidence suggests that the common belief of an approximately linear relationship among molecular weight and mobility of mid-sized DNA fragments may not be very accurate.

To remedy such error, a preferred procedure for generating a sizing function of this invention posits that a locally-quadratic relationship applies to a small region surrounding each DNA-standard fragment. More specifically, in a preferred implementation, a locally-quadratic, weighted regression approach is used to determine a sizing function that accounts for any nonlinear migration of DNA fragments and or amplicons in a gel. In this preferred approach, locally-quadratic regression is performed on subsets of DNA standards smaller than the entire complement of all DNA standards. The resultant function produces a curve that helps to more accurately and more precisely estimate the molecular weights of size-unknown amplicons.

In a preferred implementation, for each DNA fragment standard, locally-quadratic, weighted regression is performed piecewise on successive subsets of all DNA fragment standards. Preferably, the locally-quadratic fit is applied to each DNA standard such that the weighted regression is performed using less than the entire complement of DNA standards. Preferably, approximately eight or nine DNA standards are used for each locally-quadratic fit performed.

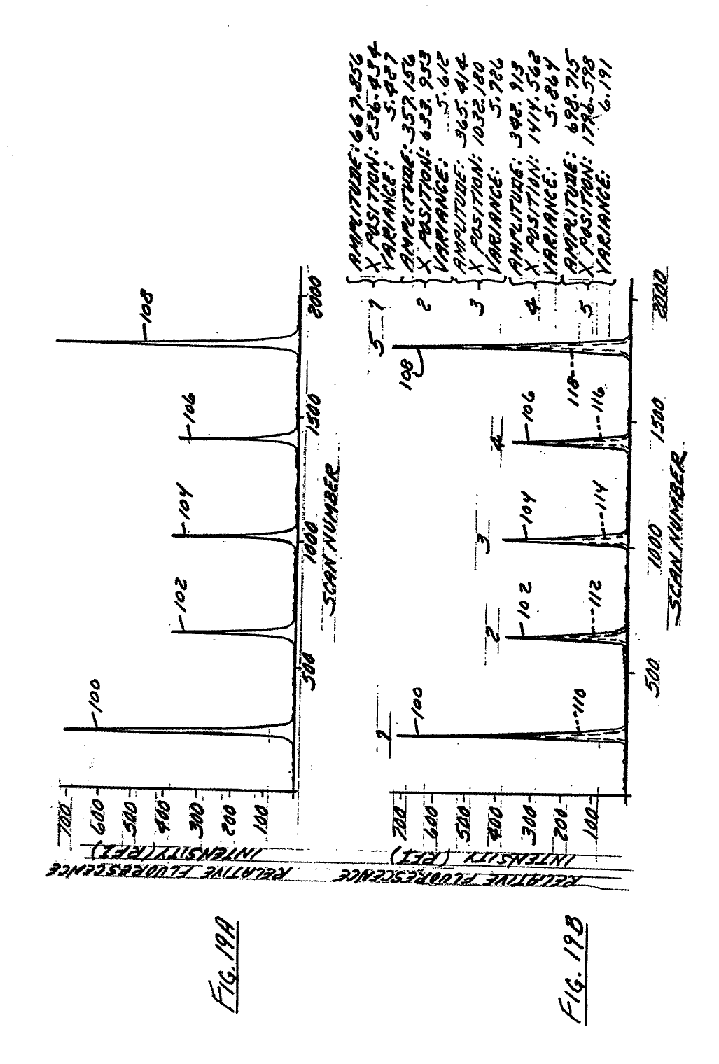

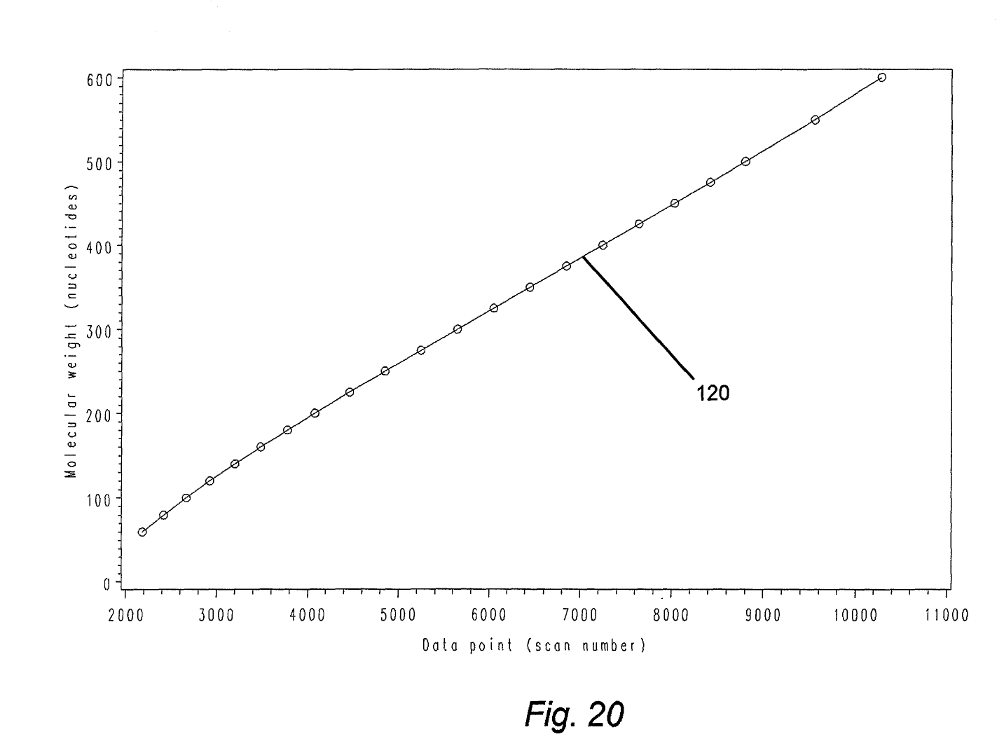

In a preferred implementation, approximately eight standards are used for each locally-quadratic fit. For example, where 22 standards are used such as with ILS-600, a locally-quadratic fit is applied to each individual DNA standard using the nearest eight DNA standards. This pattern is repeated piecewise until such a locally-quadratic fit is generated for all 22 DNA standards. As is depicted by FIG. 20, the resultant fitted data can then be used to generate a nonlinear mobility-compensated sizing curve 120.

By performing such an analysis for the DNA size standards, the resultant sizing curve generated advantageously accommodates nonlinear or anomalous DNA fragment migration while helping to achieve a high degree of molecular weight prediction for sample amplicons with precision typically less than ±0.333 nucleotide bases. In fact, the level of precision achieved is typically between ±0.05 and ±0.25 nucleotide bases. A preferred implementation of an algorithm for generating a sizing curve is discussed in more detail below.

To create a sizing function suitable for assigning molecular weights to amplicons of unknown sizes, a weighted, locally-defined quadratic weighted regression model is applied to defined neighborhoods of size-standardized DNA fragments. For example, at each DNA fragment standard position in the set of DNA standards, a polynomial of degree two (i.e. a quadratic polynomial) is fit to a subset of the entire complement of DNA standards near the fragment standard whose response is being estimated. A quadratic polynomial is fit, using weighted least-squares procedures, giving more weight to standard fragments near the DNA standard fragment whose response is being estimated and less weight to standard fragments further away. The value of the local regression function for the DNA standard being fitted is then obtained by evaluating the local polynomial using the explanatory variables computed for that standard. The regression procedure is complete after regression functions have been computed for each of the standards in the entire set of DNA standards.

The sizing function or curve is constructed by first choosing a “smoothing parameter” ƒ that denotes the fraction of DNA standards used in the computation of each locally-defined quadratic fit. The parameter ƒ is called a smoothing parameter because it controls the behavior of the regression function. For example, large values of ƒ produce very smooth functions. Small values of ƒ will more closely conform to fluctuations in the data. Using too large or too small a value of the smoothing parameter ƒ is not desirable either because the regression function will not reflect accurately subtle differences in fragment mobility (i.e. “overfit” the data) or begin to model the random error in the data, respectively. Generally, useful values of ƒ include those values of ƒ between 0.25 and 0.50.

Let q=ƒN where q is the number of DNA standards that define the neighborhood-size of the weighting function and N is the total number of DNA standards. Preferably, ƒ is a real number between (d+1)/N and 1.0, where d is the degree of the local polynomial. The value of ƒ, and therefore the value of q, is dependent on the number of DNA standards used in defining the sizing curve. For a particular number of DNA standards, ƒ preferably is chosen to minimize the residual standard error (RSE) of the weighted least-squares fit of Equation XXX below as applied piecewise to the entire set of DNA standards. For example, where the DNA standard is an ILS-600 standard, ƒ preferably is between 0.136 and 1.0. Using the criteria of RSE minimization as described above, a value of ƒ of approximately 0.4 results in a RSE of about 0.094 which is significantly less compared to any RSE resulting from an ƒ such that 0.136≦ƒ<0.4 or 0.4<ƒ≦1. Where twenty-two standards are used, q is 8.8, meaning that, after rounding down to the nearest integer, eight DNA standards will define the size of the local neighborhood in which the weighted quadratic regression is performed. Where a different DNA standard is used, routine testing and experimentation can be used to determine a suitable ƒ for that standard.

As indicated above, the weighting function gives the most weight to the DNA standards nearest to the standard position currently being estimated and reduces the weight of standards more distant from the center of the neighborhood. The use of the weighting function is based on the idea that DNA standards nearer to each other are more likely to be related to each other (e.g. in terms of mobility) in a simpler way than DNA standards much further away. Thus, standards that are most likely to follow the local model best influence the local model parameters the most. Standards that are less likely to follow the local model (because they are further away from the center of the neighborhood) influence the local parameter estimates less.

Define xᵢ as the i^{th} DNA standard fragment where i=1, 2, . . . , N. Let dᵢ be the distance (e,g, in units of scan number) from the estimated peak apex location of a standard fragment being fitted to its q^{th} nearest neighbor. The estimated peak apex location of the DNA standard fragment preferably is obtained using ODR as described above. Define the weighting function W(u) as

$$W ( u ) = \{ \begin{matrix}{( 1 - \vert u {\vert}^{3} )}^{3} & 0 \leq u < 1 \\ 0 & otherwise\end{matrix} . \qquad ( Equation XXVIII )$$

The function W(u) imposes a weight to each standard fragment in the neighborhood as a decreasing function of its distance from the center of the neighborhood. The center of the neighborhood, in this instance, is the location, xᵢ, of the standard currently being fitted. The weight contributed by the nearby standards (xₖ, yₖ), where k=1, 2, . . . , q, to that of the standard at location xᵢ being fitted is thus defined as

$${w}_{i} ( {x}_{k} ) = W ( \frac{( {x}_{i} - {x}_{k} )}{{d}_{i}} ) \qquad ( Equation XXIX )$$

where xₖ is the apex location of the nearby standard and yₖ is its known (standardized) molecular weight. The weight for a specific point in any neighborhood is obtained by evaluating the weight function at the distance between the point xₖ and the point of estimation, xᵢ, after scaling the distance so that the absolute distance over all the neighborhood points is exactly one. The fitted value for the DNA standard located at xᵢ is computed by the ordinary least-squares minimization of

$${\sum }_{k = 1}^{N} {{w}_{i} ( {x}_{k} ) [ {y}_{k} - ( {β}_{1} + {β}_{2} {x}_{k} + {β}_{3}^{2} {x}_{k} ) ]}^{2} \qquad ( Equation XXX )$$

where wᵢ(xₖ) is the weighting function of Equation XXIX. Once the best-fit estimate of parameters β₁, β₂ and β₃ are obtained (note that the parameters β₁, β₂ and β₃ of Equation XXX have no direct relationship to similarly denoted parameters in the ODR section), the molecular weight of size-unknown amplicons in each defined neighborhood are estimated by

ŷᵢ=\hat{β}₁+\hat{β}₂xᵢ+\hat{β}₃²xᵢ (Equation XXXI)

where ŷ₁ is the estimated molecular weight of the i^{th} size-unknown amplicon, xᵢ is the peak location estimate of that same amplicon that preferably is obtained by ODR as described above, and \hat{β}₁, \hat{β}₂ and \hat{β}₃ denote the least-squares estimates of β₁, β₂, and β₃ obtained from minimizing Equation XXX.

FIG. 20 illustrates an example of one such sizing curve generated using this procedure. The curve shown in FIG. 20 is produced from the results of plotting the results of Equation XXX for each of the standards in the ILS-600 DNA standard.

### VII. Amplicon Binning

After estimating a molecular weight for each size-unknown sample amplicon, a binning operation to group amplicons of similar size is preferably performed. This binning operation preferably is done because, although the aforementioned methods produce a highly accurate estimate of molecular weight for each size-unknown amplicon, some size variability nonetheless remains. This variability is manifested usually as very small differences in the estimated molecular weight of amplicons derived from the same locus thus preventing direct assignment of homology across samples. It is therefore desirable to bin or group similarly-sized amplicons and thus define groups of amplicons based on homology. It is posited that amplicons exhibiting estimated molecular weights that differ only slightly are in fact identically sized, (i.e. homologous), across samples. Preferably, the binning procedure eliminates any remaining variability attributable to small differences, typically less than 0.333 nucleotide bases, in the estimated molecular weight. This procedure also has the advantage of providing a means by which to assign amplicons to discrete and homologous groups across multiple samples. A preferred implementation of a procedure for binning the molecular weights of amplicons is discussed in more detail below. Other binning procedures can be used.

Although each amplicon now has an estimated size assigned to it, these molecular weights are [numerically] continuously valued. In other words, due to variability in assigning a molecular weight to any amplicon, the estimated size of the amplicon is unlikely to be an exact integer reflecting the precise size of the amplicon, for example, in nucleotide bases. Therefore, the estimated amplicon sizes must be transformed into a discrete representation of an amplicon at a specific locus. This procedure is necessary to make assign homology to homologous amplicons across other samples.

In a preferred implementation, for each processed sample, Sᵢ, i=1, 2, . . . , N, the estimated molecular weights of the j^{th} amplicon Sᵢⱼ, j=1, 2, . . . , nᵢ^{*}, are arranged in ascending order

$${S}_{ij} = \{ \begin{matrix}{S}_{11} < {S}_{12} < … < {S}_{1 j} \\ {S}_{21} < {S}_{22} < … < {S}_{2 j} \\ … \\ {S}_{i1} < {S}_{i2} < … < {S}_{ij}\end{matrix} \qquad ( Equation XXXII )$$

where N is the total number of samples and nᵢ^{*} is the total number of amplicons in each sample Sᵢ. Due to the possibility of polymorphism between samples, for example, in the case of a homozygote null in one or more samples, the value of n* for a specific sample Sᵢ does not have to be equal over all N samples. For each amplicon represented as a molecular weight in Sᵢⱼ, any two amplicons with molecular weights, Sᵢⱼ, Sᵢⱼ^{*}, are defined to be identically-sized if

|Sᵢⱼ−Sᵢⱼ^{*}|≦ε (Equation XXXIII)

where ε is the largest residual of all molecular weight estimates derived from Equation XXX above. To transform these molecular weights into discrete representations of homologous amplicons, a new matrix M is created and mapped onto the matrix Sᵢⱼ where each element in Mᵢⱼ is defined as

$${M}_{ij} = \{ \begin{matrix}1 & \vert {S}_{ij} - {S}_{ij}^{*} \vert \leq ϵ \\ 0 & otherwise\end{matrix} . \qquad ( Equation XXXIV )$$

The end result of this procedure is a matrix of ones and zeros indicating whether or not any amplicon represented by its molecular weight in Sᵢⱼ has a homologous representation in a given sample i at position j in matrix Mᵢⱼ. Because the presences of all homologous amplicons across all samples are now accounted for, the value of nᵢ^{*} must be equal across all N samples in matrix Mᵢⱼ.

The jₜₕ column, j=1, 2, . . . , n*, in matrix Mᵢⱼ may be conveniently referred to as a “locus” using classical genetic terminology. Defined in this manner, matrix Mᵢⱼ is equivalent to an indicator of amplicon phenotype (i.e. presence or absence) at each locus. Because the presence of an amplicon is indicative of either a homozygote present or heterozygote genotype, matrix Mᵢⱼ is therefore a dominant representation for each amplicon genotype.

In Mᵢⱼ, any locus j,j=1, 2, . . . , n* is defined putatively monomorphic, pMMₖ, k=1, 2, . . . , m, if at all i, Mᵢⱼ=1. If at any i, Mᵢⱼ≠1, then locus j is defined polymorphic, PM₁, l=1, 2, . . . , p. In a preferred implementation, each locus is uniquely defined by computing the average molecular weight of all amplicons present at that locus. This procedure preferably is accomplished by first referencing the presence of each amplicon in Mᵢⱼ(i.e. each Mᵢⱼ−1) back to the original molecular weight table, Sᵢⱼ. When computing the average molecular weight for each locus, null homozygotes in Sᵢⱼ (i.e. each Mᵢⱼ=0) are excluded from the calculation.

### VIII. Estimation of Amplicon Spectral Intensity Introduction

As described above, the matrix Mᵢⱼ provides information concerning only the presence and/or absence of amplicons at a given locus in a given sample. This information is limited to identifying only the phenotype (i.e. presence and/or absence) of the amplicon and is therefore dominant data as genotypes for each amplicon remain unknown. The procedures described below provide a preferred methodology to enable the recovery of genotypic information previously confounded as an expression of dominant-only phenotypic data. The overall result of this procedure is a transformation of the matrix of dominant-only phenotypic data into a matrix of co-dominant genotypic data where each amplicon has assigned to it a specific genotype.

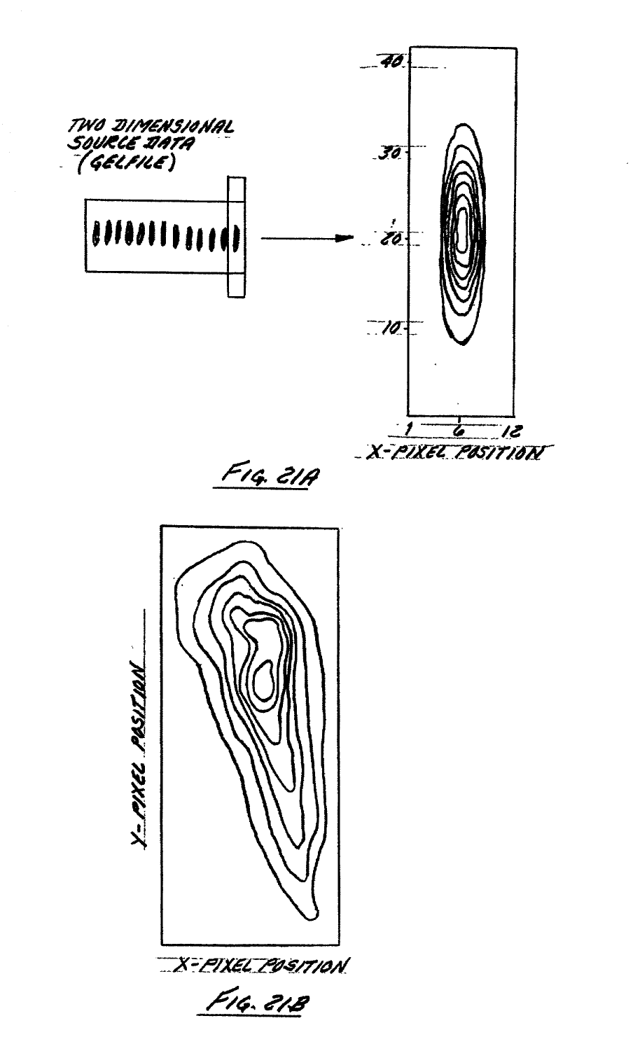

Referring additionally to FIGS. 21A, 21B, 22A and 22B, during gel electrophoresis, individual DNA molecules in a gel or polymer matrix undergo random movements over time due to both concentration and electrically-mediated displacement effects. Because of random statistical fluctuations in these complicated processes, the DNA molecules experience directional and displacement asymmetry, so that they spread outward from a uniformly distributed starting point. In this way, an initially very narrow band of identically-sized DNA molecules (e.g. an amplicon) widens into a characteristic Gaussian-shaped profile. See, e.g., FIGS. 17A-19B and FIGS. 21A-22B.

This physical phenomenon presents a convenient situation in which to mathematically model the distribution of spectral intensity in an amplicon and therefore estimate the amount of DNA molecules present in the amplicon. Because each DNA molecule is labeled only with a single fluorophore, and only fluorophore-labeled DNA molecules are detected, the total spectral intensity will be directly proportional to the actual number of fluorophore-labeled DNA molecules in the amplicon. Thus, the total amount of spectral intensity observed in an amplicon provides a robust measure of the total number of fluorophore-labeled DNA molecules in that amplicon. The ability to estimate the relative number of DNA molecules in an amplicon allows inferences concerning the number of alleles from which the PCR-generated amplicon was originally derived. With this information, genotypes may be accurately assigned to individual amplicons.

It should be noted that others have tried to estimate the total spectral intensity of amplicons by simply adding the recorded signal intensities of fluorophore-labeled DNA molecules. This approach is not always preferred because: (1) numerous sources of spectral noise exist and contribute significantly to spectral-intensity measurement error, (2) most fluorescence detectors have relatively low sampling rates and provide only a coarse sample of spectral emissions at any given position in the gel, such as is depicted in FIG. 22A, (3) the data available for use may have been processed using unknown procedures, and (4) no intrinsically-defined method to delimit the boundaries of an amplicon exist other than perhaps an arbitrary “cutoff” value.

A procedure that preferably is used in a method of the invention, described below, can be used to overcome these limitations. First, a mathematical model is provided that describes the effects on an amplicon of (1) passive diffusion and (2) the additional additive effects of electrically-mediated displacement. Based on this model, a parametric statistical model is then presented that preferably is suitable for use as a procedure implemented in software that estimates the total amount of spectral intensity present in an amplicon 56 (FIG. 1). This procedure is ultimately well suited for use in estimating the spectral intensity of an amplicon and therefore the relative number of fluorophore-labeled DNA molecules in that amplicon. Ultimately, the relative number of DNA molecules in an amplicon is used to infer allele dosage (2, 1, or 0 copies), which in turn is a direct indicator of genotype (homozygote present, heterozygote, or homozygote absent [null]).

### Effects of Gel Electrophoresis on an Amplicon

### I. Passive Diffusion

It is assumed that the gel or polymer matrix in which passive diffusion of a single-stranded DNA fragment occurs is isotropic and effectively either one-dimensional, such as where capillary electrophoresis is used, or two-dimensional, such as where a slab gel electrophoresis is used. The change in concentration from an initially very narrowly deposited source of identically-sized DNA molecules (e.g. an amplicon deposited into a sample well) is described in one dimension (x) by

$$\frac{\partial C}{\partial t} = {D}_{p} ( \frac{{\partial }^{2} C}{\partial {x}^{2}} ) \qquad ( Equation XXXV )$$

and in two dimensions (x and y) by

$$\frac{\partial C}{\partial t} = {D}_{p} ( \frac{{\partial }^{2} C}{\partial {x}^{2}} + \frac{{\partial }^{2} C}{\partial {y}^{2}} ) . \qquad ( Equation XXXVI )$$

For the above equations, C is the concentration of the diffusing DNA molecules, t refers to time, and D_{P} is a constant describing the passive diffusion of single-stranded DNA molecules in the defined isotropic matrix. For the one-dimensional diffusion case of Equation XXXV, C is a function of scan number or location x (e.g. the direction of electrophoresis), and x, y (e.g. the dimension containing the sample wells and the dimension of electrophoresis, respectively), for the two-dimensional diffusion case of Equation XXXVI. For the two-dimensional case, x specifically refers to the channel numbers spanned by each sample well, and y refers to scan number or location along a particular channel in the direction of electrophoresis. Preferably, D_{P}, defines the passive diffusion rate of single-stranded DNA molecules in a polyacrylamide gel or other defined gel matrix. For discussion contained herein, D_{P} is assumed to be constant for a given length of ssDNA. Methods of determining D_{P} are known in the literature and are dependent upon factors that include: (1) the porosity of the gel matrix, (2) the strength of the electric field used, and (3) the length of the DNA molecule. Routine experimentation can be performed to determine D_{P} and estimates are also available in the literature.

For the one-dimensional case, the initial deposition of an amount of identically-sized DNA molecules onto a polyacrylamide gel or other gel matrix is defined as an idealized unit impulse function, δ(x−a). This function describes the properties of applying a unit amount of identically-sized DNA molecules onto the gel matrix at position x=a (e.g. the top portion of the gel matrix) as unity but being zero for all other values of x

δ(x−a)=0x≠a. (Equation XXXVII)

Therefore, at time t=0, the origin of sample deposition at x=a is characterized as an infinitely narrow band of identically-sized DNA molecules of unit area

$${\int }_{- ∞}^{+ ∞} δ ( x - a ) ⅆ x = 1. \qquad ( Equation XXXVIII )$$

Given these initial conditions, the fragment band will evolve by diffusion processes into a Gaussian shape.

Ordinary differentiation of Equation XXXV provides a solution for diffusion of DNA molecules in one dimension

$$C = \frac{const}{\sqrt{t}} exp ( - {x}^{2} / 4 {D}_{p} t ) \qquad ( Equation XXXIX )$$

where const is a constant related to the amount of DNA originally deposited onto the gel matrix. Equation XXXIX is symmetrical with respect to x=0 and tends toward 0 as x→∞ either positively or negatively for t>0. For t=0, x=0 everywhere except at x=0 where the function is described by the delta function of Equation XXXVIII. To evaluate the constant, const, consider the total amount of identically-sized DNA molecules deposited onto the gel matrix is defined as

$$M = {\int }_{- ∞}^{+ ∞} C ⅆ x . \qquad ( Equation XL )$$

Substituting −x²/4D_{P}t= [symbol] ², dx=\sqrt{4D_{P}t}d [symbol] provides a means for calculating M by

$$\begin{matrix}M = const \sqrt{4 {D}_{p}} {\int }_{- ∞}^{+ ∞} exp ( - {}^{2} ) ⅆ \\ = const \sqrt{4 π {D}_{p}} .\end{matrix} \qquad ( Equation XLI )$$

Equation XLI shows that the amount M of DNA molecules diffusing remains constant and equal to the originally deposited amount at time t=0 at position x=a. In other words, Equation XXXIX normalizes to unit area (i.e. it applies to, for example, one DNA molecule per unit area) when M=const\sqrt{4πD_{P}}. Therefore, on substituting for const from Equation XLI in Equation XXXIX, the solution to the change in concentration in an amount M of identically-sized DNA fragments at a given time t and position x in one dimension is

$$C = \frac{M}{\sqrt{4 {πD}_{p} t}} {exp}^{- ( \frac{{( x - {x}_{0} )}^{2}}{4 {D}_{p} t} )} \qquad ( Equation XLII )$$

and positions x, y in two dimensions is

$$C = \frac{M}{\sqrt{4 {πD}_{p} t}} {exp}^{- ( \frac{{( x - {x}_{0} )}^{2}}{4 {D}_{p} t} - \frac{{( y - {y}_{0} )}^{2}}{4 {D}_{p} t} )} . \qquad ( Equation XLIII )$$

Note that radial symmetry in the two-dimensional diffusion process is not assumed.

Diffusion of the DNA molecules occurs because they (1) drift under random kinetic motion and (2) more of them move from a high concentration region, located at the center of the amplicon, to a low concentration area that is located away from the center of the amplicon. The mean distance in which an individual DNA molecule diffuses is zero because diffusion in the −x and +x directions for the one-dimensional case is equally probable. However, the mean square distance is not zero:

$$\begin{matrix}{σ}_{x}^{2} = {\int }_{- ∞}^{+ ∞} {x}^{2} \frac{M}{\sqrt{4 {πD}_{p} t}} {exp}^{( - \frac{{( x - {x}_{0} )}^{2}}{4 {D}_{p} t} )} ⅆ x & ( Equation XLIV ) \\ = 2 {D}_{p} t . & ( Equation XLV )\end{matrix}$$

Equation XLV shows that for passive diffusion, σₓ², which corresponds to the peak variance or width, is directly proportional to time, t, provided D_{P} remains constant. Similar arguments are also directly applicable to the two-dimensional case of diffusion.

### Effects of Gel Electrophoresis on an Amplicon

### II. Active Displacement

Random events other than passive diffusion of DNA molecules also contribute substantially to the effective diffusion rate of a DNA molecule in an isotropic gel matrix. In gel electrophoresis systems, DNA molecules are also actively transported by an electrical field through a porous gel or polymer matrix. The molecular conductance pathways change randomly in response to random changes in the composition of the gel or polymer matrix; DNA molecules may be forced to stop and diffuse laterally before resuming forward migration. Hence, a molecule of DNA will undergo many changes in velocity, and thus position, thereby contributing to an increase in the effective diffusion rate. Considering only one-dimensional molecular displacement, incorporating the effects of electrically-mediated displacement velocities into Equation XXXV above leads to

$$\frac{\partial C}{\partial t} = - {V}^{′} ( \frac{\partial C}{\partial x} ) + {D}_{P} ( \frac{{\partial }^{2} C}{\partial {x}^{2}} ) \qquad ( Equation XLVI )$$

where V′ is the displacement velocity of the DNA molecule in the gel matrix. Note that V′ will also experience random velocity fluctuations and therefore contribute an additional variance component to the effective diffusion rate. To account for these random velocity fluctuations, the displacement velocity V′ can be broken into two parts

V′=V+V_{A} (Equation XLVII)

where V is the average displacement velocity (assumed to be constant) and V_{A} incorporates the random component of velocity fluctuations. Therefore,

$$\frac{\partial C}{\partial t} = - V ( \frac{\partial C}{\partial x} ) - {V}_{A} ( \frac{\partial C}{\partial x} ) + {D}_{P} ( \frac{{\partial }^{2} C}{\partial {x}^{2}} ) . \qquad ( Equation XLVIII )$$

The random nature of V_{A} thus contributes an additional diffusion term

$$- {V}_{A} ( \frac{\partial C}{\partial x} ) = {D}_{A} ( \frac{{\partial }^{2} C}{\partial {x}^{2}} ) \qquad ( Equation XLIX )$$

where D_{A} is a constant describing the diffusion of a DNA molecule attributable to active (e.g. electrically mediated) displacement. Substituting Equation XLVIII into Equation XLVII leads to

$$\frac{\partial C}{\partial t} = - V ( \frac{\partial C}{\partial x} ) + ( {D}_{A} + {D}_{P} ) ( \frac{{\partial }^{2} C}{\partial {x}^{2}} ) . \qquad ( Equation L )$$

Equation L is of the same form of Equations XXXV and XXXVI and yields similar Gaussian solutions as presented by Equations XLII and XLIII. The effective diffusion rate is enhanced because the coefficient ∂²C/∂x² of is now the sum of two diffusion terms

D_{Total}=D_{P}+D_{A}. (Equation LI)

One term, D_{P}, represent passive diffusion of the DNA molecule and the other term, D_{A}, represents diffusion due to active (e.g. electrically mediated) displacement of the DNA molecule. Similar to Equation XLV, the total effective diffusion coefficient, D_{Total}, of each DNA amplicon acquires a variance in the form of

σₓ²=2D_{Total}t (Equation LII)

that is also proportional to time, t, provided that D_{Total} remains constant. For example, at constant V, the distance, x, traversed by the amplicon in time, t, is simply x=Vt. Substituting t=x/V into Equation LII leads to

$${σ}_{x}^{2} = ( \frac{2 {D}_{Total}}{V} ) x . \qquad ( Equation LIII )$$

Equation LIII shows that σₓ², based on the combined effects of passive and active diffusion, is also directly proportional to the amplicon migration distance, x. Identical arguments can easily be made for the same passive and active diffusion effects in two-dimensional systems.

### Effects of Gel Electrophoresis on an Amplicon

### III. Detector Effects

It is a well-known that the separation of DNA molecules in a gel matrix occurs because of different migration velocities attributable to DNA molecules of distinct sizes. For example, small DNA molecules move through a gel matrix at greater velocities than do larger DNA molecules. This phenomenon creates a third source of “apparent” diffusion due to differences in the velocity by which a DNA molecule passes by the detection region of the detector. For example, many automated DNA sequencers (e.g. ABI capillary and slab-gel sequencers) operate in what is known as a “finish-line” mode where differently-sized DNA molecules migrate the same distance but at different times.

When applying finish-line electrophoresis, small-sized amplicons will move across the detector region relatively quickly while larger-sized amplicons will pass by the detector region more slowly. Consequently, due to the size-dependent migration velocities of DNA molecules, the apparent magnitude of diffusion will be larger for amplicons of larger size. In other words, the magnitude of σₓ² and σ_{y}², as predicted by the aforementioned one- and -two dimensional diffusion models, respectively, will typically be an underestimate of the observed values of σₓ² and σ_{y}² because of the time a particular DNA molecule takes to pass the detection region.

Provided the gel matrix and electrophoresis conditions remain constant, the observed time-delays for identically-sized DNA molecules should be identical or nearly identical across samples and across electrophoresis runs. However, in situations where this assumption cannot be made, it is desirable to compensate for this effect. A preferred approach is to convert the observed values of σₓ² and/or σ_{y}² on the original “time-delayed” scale to actual values of amplicon width (in units of length) by multiplying with the velocity of the migrating amplicon. For example, the velocity at which an amplicon moves through a gel matrix may be computed by the simple relationship, ν=L/t, where ν is the velocity of the DNA molecule (e.g. amplicon), L is the distance from the sample-well (or injection position) to the center of the detection region, and t is the time it takes for the amplicon to reach the center of the detection region. In ABI-based detection systems, the distance from the sample-well to the detection window is known. The time it takes the amplicon to reach the detection window may also be determined by simply converting the unit of scan number to a unit of time.

It may be desirable to calculate the true diffusion coefficients, e.g. to compare the observed results to theoretical expectations. In a preferred implementation, the total variance in amplicon width is rewritten as a sum of the variance in amplicon width due to electrophoresis, σ_{EIectrophoresis}², and variance in amplicon width as a result of additional detector delay, σ_{Detector}², leading to σ²−σ_{Electrophoresis}+σ_{Detector}². By assuming that the additional increase in observed amplicon width is entirely due to diffusion, the true diffusion coefficient is related to the additional amplicon width as σ_{Detector}²=2D_{Total}t_{delay}, where t_{delay} is the delay time of an amplicon in the detector region. Thus, the true diffusion coefficient may be estimated, for example, by plotting the amplicon-width variances as a function of the delay times; the diffusion coefficient is equal to 0.5 multiplied to the slope of a linear, least-squares fit to the amplicon widths.

### Estimation of Spectral Intensity in an Amplicon

### I. Gaussian model

The diffusion model described above provides a means by which to accurately model the distribution of DNA molecules comprising an amplicon in an electrophoretic system. In turn, the same model provides the mathematical criteria necessary to compute the total amount of spectral intensity in an amplicon from photometric data. This procedure not only provides a relative estimate of the number of DNA molecules in an amplicon, but also provides a means to infer the number of DNA templates from which an amplicon was originally derived. Together, these capabilities ultimately make available a means to assign genotypes to specific amplicons where none could be designated previously. A preferred implementation of a procedure for estimating spectral intensity 56 (FIG. 1) is described herein further detail.

Dividing Equation LII (and its two-dimensional form) into Equations XLII and XLIII, respectively, and replacing the amplitude variable M/\sqrt{4πD_{Total}t} with the variable I, solvable parametric models of DNA molecules comprising an amplicon, C(x), in an isotropic medium are obtained for the one-dimensional case

$$C ( x ) = I {exp}^{- ( \frac{{( x - {x}_{0} )}^{2}}{2 {σ}_{x}^{2}} )} \qquad ( Equation LIV )$$

and for the two-dimensional case

$$C ( x , y ) = I {exp}^{- ( \frac{( x - {x}_{0} )}{2 {σ}_{x}^{2}} - \frac{( y - {y}_{0} )}{2 {σ}_{y}^{2}} ) .} \qquad ( Equation LV )$$

Equations LIV and LV may be fitted to photometric data preferably using nODR or nOLS procedures as described above. Preferably, estimates of peak amplitude, peak location, and amplicon variance that have been previously obtained with nODR or nOLS techniques or other methods described herein are used as starting values for the preferred nODR minimization procedure.

In the one-dimensional case, for example, the value of I corresponds to peak amplitude (e.g. in units of RFI), x₀ is the true peak location (e.g. in units of scan number), x is an observed location in the set of photometric data (e.g. in units of scan number), and σₓ² is the amplicon variance (e.g. in units of scan number, either corrected or uncorrected for detector time-delay effects). In a preferred implementation, it is desired to use estimates of these variables, namely parameters I=β₁, x₀=β₂, and σₓ²=β₃, previously generated using Equation XXV above. In that case, Equation LIV is rewritten to

$$C ( x ) = {β}_{1} {exp}^{- ( \frac{{( x - {β}_{2} )}^{2}}{2 {β}_{3}^{2}} ) .} \qquad ( Equation LVI )$$

For the two-dimensional case, the definition and units of the amplitude I remain the same as in the one dimensional case. However, the definitions and units of x₀, x and σₓ² must be adjusted to reflect changes due to the addition of another dimension. In two dimensions, x₀ is the true peak location in the first dimension (e.g. in units of channel number), x is an observed location in the first dimension in the set of photometric data (e.g. in units of channel number), and σₓ² is the amplicon variance in the first dimension (e.g. in units of channel number). Note that there is no delay response associated with the detector region in the x dimension of channel number.

To mathematically accommodate the second dimension, three additional variables are necessary: y₀ is the true peak location (e.g. in units of scan number), y is an observed location in the set of photometric data (e.g. in units of scan number), and σ_{y}² is the amplicon variance (e.g. in units of scan number either corrected or uncorrected for detector time-delay effects). In a preferred implementation, it is desired to use estimates of parameters β₁, β₂, and β₃ estimated from Equation XXV as starting values for parameters I=β₁, y₀=β₄, and σₓ²=β₅in the two dimensional case, respectively. Note that the x dimension coordinates and parameters were simply transposed to the y dimension coordinates for parameters β₄ and β₅.

The remaining variables in the second dimension, x₀ and σₓ² are estimated by parameters β₂ and β₃, respectively. Estimates for starting values for these two additional parameters are obtained as follows: In a preferred implementation, β₂ preferably is initially estimated as the channel number nearest to the intersection of the tracking spline that bisects the amplicon being fitted. In a preferred implementation, β₅ is preferably estimated by directly measuring the width of the sample well, in units of channel numbers. For example, 64-lane combs (part no. CAG64-020) manufactured by The Gel Company (665 Third Street, Suite 240, San Francisco, Calif. 94107) have a well-width of approximately 4-5 channels. If other sample-well combs are used, and/or used on other detectors with differing numbers of channels, routine experimentation can be performed to determine the width of the sample wells in units of channels. Preferably, as discussed above, estimates of all parameters in either one or two dimensions are determined prior to their use in this part of the procedure as they are highly accurate starting values and will allow quick convergence of the fitting algorithm.

In a preferred implementation of the two-dimensional case, estimates of these variables, as reflected in parameters β₁ . . . β₅, are substituted into Equation LV above. In that case, Equation LV is rewritten as

$$C ( x , y ) = {β}_{1} {exp}^{- ( \frac{{( x - {β}_{2} )}^{2}}{2 {β}_{3}^{2}} - \frac{{( y - {β}_{4} )}^{2}}{2 {β}_{5}^{2}} ) .} \qquad ( Equation LVII )$$

Estimates of the parameters in either the one- or two-dimensional case are made by fitting Equations LVI or LVII to the processed photometric data, respectively, preferably using nonlinear ODR or nonlinear OLS procedures as described above.

To compute the total amount of spectral intensity present in an individual amplicon, Equation LIV is integrated from −∞→+∞ to yield an analytical solution for the one-dimensional case

$$\begin{matrix}{\int }_{- ∞}^{+ ∞} C ( x ) ⅆ x = I {\int }_{- ∞}^{+ ∞} {exp}^{- ( \frac{{( x - {x}_{0} )}^{2}}{2 {σ}_{x}^{2}} )} ⅆ x & ( Equation LVIII ) \\ = I \sqrt{2 {πσ}_{x}^{2}} & ( Equation LIX )\end{matrix}$$

Substituting the estimated values for peak amplitude, \hat{β}₁, and amplicon variance, \hat{β}₃, into Equation LVIII yields the following

$${\int }_{- ∞}^{+ ∞} C ( x ) ⅆ x = {\hat{β}}_{1} \sqrt{2 π {\hat{β}}_{3}^{2}} . \qquad ( Equation LX )$$

Equation LX allows direct calculation of the spectral intensity of the entire amplicon as reflected by the parameters which minimize the error of the fitting procedure described above. Integration of Equation LV yields a similar analytical solution for the two-dimensional case

$$\begin{matrix}{\int }_{- ∞}^{+ ∞} {\int }_{- ∞}^{+ ∞} C ( x , y ) ⅆ x ⅆ y = I {\int }_{- ∞}^{+ ∞} {\int }_{- ∞}^{+ ∞} {exp}^{- ( \frac{( x - {x}_{0} )}{2 {σ}_{x}^{2}} - \frac{( y - {y}_{0} )}{2 {σ}_{y}^{2}} )} ⅆ x ⅆ y & ( Equation LXI ) \\ = I \sqrt{4 {π}^{2} 2 {σ}_{x}^{2} 2 {σ}_{y}^{2}} . & ( Equation LXII )\end{matrix}$$

Substituting the estimated values for peak amplitude, \hat{β}₁, peak variance in the x dimension, \hat{β}₃, and peak variance in the y dimension, \hat{β}₅, into Equation LXI yields the following equation

$${\int }_{- ∞}^{+ ∞} {\int }_{- ∞}^{+ ∞} C ( x , y ) ⅆ x ⅆ y = {\hat{β}}_{1} \sqrt{4 {π}^{2} {\hat{β}}_{3}^{2} {\hat{β}}_{5}^{2}} . \qquad ( Equation LXIII )$$

Similarly to the one-dimensional case, Equation LXIII allows direct calculation of the spectral intensity of the entire amplicon as reflected by the parameters which minimize the error of the fitting procedure described above.

Once spectral intensities have been estimated for all amplicons of interest, all elements in the putative monomorphic/polymorphic matrix where Mᵢⱼ=1 are substituted with the value of integrated spectral intensity corresponding to the mapped amplicons in matrix Sᵢⱼ.

For the vast majority of amplicons, such as those depicted in FIG. 21A, the preceding implementation of spectral intensity estimation provides extremely accurate estimates of total spectral intensity. However, situations do arise, especially in two-dimensional electrophoretic systems, where DNA molecules do not always follow the aforementioned diffusion processes. Typically, this occurs where the amplicon is an anomalously-shaped amplicon, such as is depicted in FIG. 21B. This phenomenon most commonly results from: (1) deformations in the sample loading well, (2) deformation of the gel or polymer matrix at the point of initial fragment deposition, (3) anisotropy of the gel or polymer matrix, and/or (4) physical debris within the gel matrix including air bubbles. In such cases, the model based on characteristics of ssDNA diffusion in a gel matrix, as described above, does not always yield a good fit to the data. This is often observed as an uncharacteristically large residual sum-of-squares of the model fit. In such cases, it is desirable to estimate the spectral intensity of an amplicon using a less restrictive (i.e. more general) surface-fitting model. A preferred implementation of such a model is described below.

### Estimation of Amplicon Spectral Intensity

### II. Thin-Plate Spline (TPS) Model

In another preferred procedure for estimating the spectral intensity of amplicons, a highly generalized surface-fitting procedure employing thin-plate splines (TPS) can be implemented to accommodate anomalously-shaped DNA fragments. This type of surface-fitting methodology has an important advantage over the Gaussian model described above in that it does not depend on any intrinsic characteristics of (1) the electrophoretic device, (2) the separation matrix, or (3) the general behavior of DNA molecules in the separation matrix. Therefore, the TPS surface-fitting procedure can accommodate not only patterns resulting from the diffusion processes described above, but also almost any possible deformation that may occur during electrophoresis.

Briefly, TPS surface-fitting procedures can be considered as generalizations of standard multivariate linear regression procedures, except that the parametric model of Gaussian diffusion described above is replaced by a non-parametric function that preferably is a spline. Such TPS functions have certain desirable features, such as control over how smooth the function will fit to the observed data. The degree of smoothness of the fitted TPS function is determined by minimizing a measure of the predictive error of the fitted surface. This measure is preferably determined by generalized cross-validation techniques or by routine experimentation. Once the TPS is fitted to the observed spectral intensity data, the total spectral intensity may be computed by applying standard integration procedures to the fitted surface. Application of TPS functions fitted to photometric data as used herein has consistently yielded smaller variances in estimated spectral intensity than the preferred one- and two-dimensional Gaussian procedures discussed above. A more detailed description of a preferred implementation of the TPS surface-fitting procedure is given below. Although we restrict our discussion of TPS fitting to two dimensions, its use in one dimension is also possible and preferred when the one-dimensional Gaussian procedure does not yield acceptable results.

A TPS function is a generalized smoothing function that interpolates the surface of regularly or irregularly spaced data of the form

{x₁, x₂, ƒ(x₁, x₂)=ƒ(x₁)}. (Equation LXIV)

Restricting the TPS function to two dimensions, consider a two-dimensional independent variable X=(x₁, x₂) and assume observations are from the additive model

Yᵢ=ƒ(x₁)+εᵢ, for 1≦i≦n (Equation LXV)

where Yᵢ is the observed response at the i^{th} combination variable xᵢ, ƒ is the function of interest, and n is the number of observations. In practice, the true function ƒ is unknown, but is assumed to be smooth. The random error components, εᵢ, are assumed to be independent and normally distributed as N(0, σ_{ε}_{ᵢ}²). The goal of applying a TPS is to build a surface function ƒ(xᵢ) whose graph passes near or through all the observed landmark points {x₁, x₂} while minimizing the function

$${J}_{2} ( f ) = {\int }_{- ∞}^{+ ∞} {\int }_{- ∞}^{+ ∞} ( {( [ \frac{{\partial }^{2} f}{\partial {x}_{1}^{2}} ] )}^{2} + {2 [ \frac{{\partial }^{2} f}{\partial {x}_{1} \partial {x}_{2}} ]}^{2} + [ \frac{{\partial }^{2} f}{\partial {x}_{2}^{2}} ] ) ⅆ {x}_{1} ⅆ {x}_{2} . \qquad ( Equation LXVI )$$

Equation LXVI is often referred to as the bending energy of the TPS function as it reflects the overall degree of curvature imposed by the fit of the function to the given surface. With reference to the accompanying invention, xᵢ may be interpreted as pixel coordinates {xᵢ, yᵢ} and ƒ(xᵢ) as relative fluorescence intensity {zᵢ} preferably in two-dimensional gel systems. For tabulated coordinates, {x₁ , x₂, ƒ(xᵢ)}, describing a single amplicon derived from a two-dimensional gel system, the TPS minimizing function is of the form

$${S}_{λ} ( f ) = \frac{1}{n} {\sum }_{i = 1}^{n} {( {y}_{i} - f ( {x}_{i} ) )}^{2} + λ {J}_{2} ( f ) \qquad ( Equation LXVII )$$

where λ>0 is a smoothing parameter describing the roughness (cf. precision) of the TPS surface fit. In other words, the parameter λ controls the balance between the goodness of fit and the smoothness of the final TPS surface-fitting function. For example, when λ=0, the estimate of ƒ(xᵢ) is equivalent to exact interpolation of the data when the graph of the TPS function passes through every point in ƒ(xᵢ). Given the presence of noise in the spectral measurements of fluorescence intensity, exact interpolation is usually not desired. Increasing the value of λ allows greater flexibility of the TPS fit and provides an effective means by which to accommodate spectral noise.

Choosing the magnitude of the smoothing parameter λ greatly affects the final character of the TPS surface fit and thus requires careful selection. An objective methodology to select λ relies on generalized cross-validation (GCV) procedures, which approximate the predicted mean square error of the TPS-fitted function. Let A(λ) denote a n×n smoothing matrix that maps the observed data vector y onto the predicted values of the TPS fit

(ƒ(x₁), ƒ(x₂), . . . , ƒ(xₙ))^{T}=A(λ)y. (Equation LXVIII)

where T indicates the transpose of the matrix. The trace of A(λ) is interpreted as a measure of the number of effective parameters present in the TPS representation of the surface. The residuals of the TPS fit are given by (I−A(λ))y with n−traceA(λ) reflecting the degrees of freedom associated with the residual error. The GCV function is thus defined as

$$V ( λ ) = \frac{( 1 / n ) {y}^{T} ( I - {A ( λ )}^{T} W ( I - A ( λ ) ) y}{{( 1 - trace ( A ( λ ) ) / n )}^{2}} . \qquad ( Equation LXVIX )$$

The value of λ that minimizes the GCV function yields the TPS model fit that is least affected by any single data point. Once \hat{λ} is found, the variance of the surface fit, σ², may be estimated by

$${\hat{σ}}_{\hat{λ}}^{2} = \frac{{{y}^{T} ( I - A ( \hat{λ} ) )}^{T} W ( I - A ( \hat{λ} ) ) y}{n - trace ( A ( \hat{λ} ) )} \qquad ( Equation LXX )$$

and is analogous to the estimate of the residual variance from classical linear regression. The use of the GCV function to select λ is based on asymptotic theory. Therefore, it is important that sample sizes contain enough observations to provide a sufficient amount of information so as to be able to discriminate between actual signal and background noise. In practice, experience suggests that the signal-to-noise ratio of data actually obtained is more than adequate to provide reliable estimates of λ using the GCV procedure. Once the TPS function is fitted to the data, the spectral intensity is estimated using standard integration techniques.

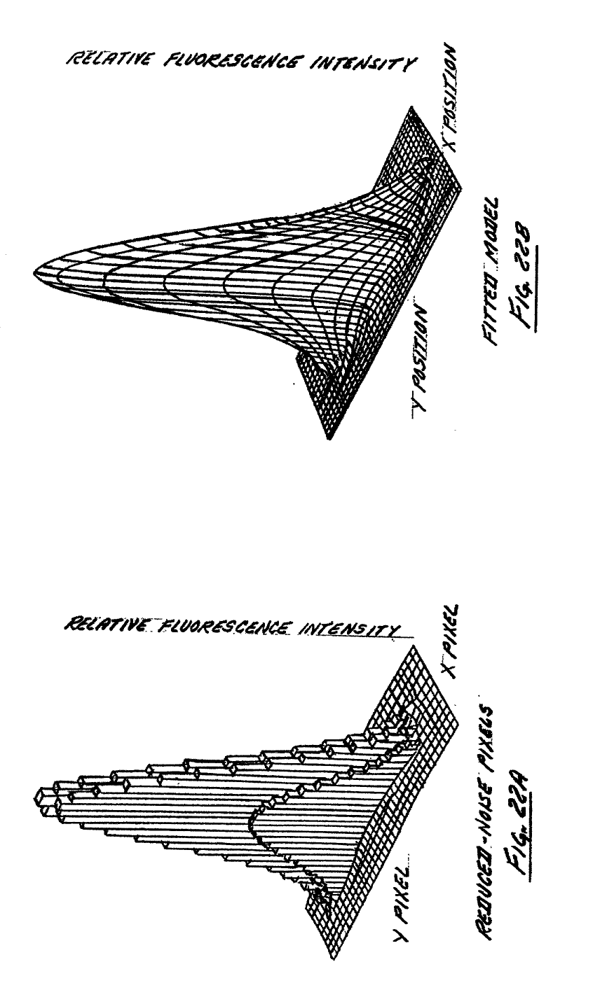

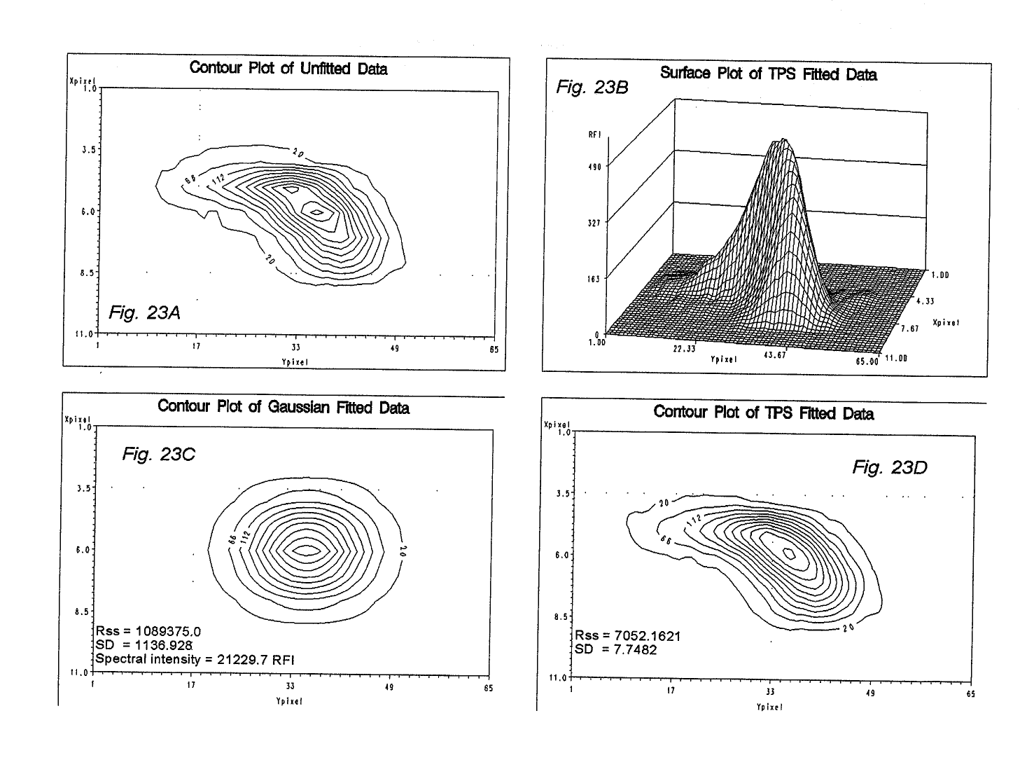

An example of this preferred implementation of a TPS-fitted surface to an amplicon in two dimensions is shown in FIG. 23. A contour plot of spectral intensity for a distorted amplicon (also see FIG. 21B) is shown in FIG. 23A. A TPS-fitted surface was computed as described above, using a value of λ=0.0001, and is shown in FIG. 23B. Compared to the Gaussian fit of a non-distorted amplicon shown in FIG. 22B, the TPS-fitted surface is not symmetrical and conforms much more closely to the original distribution of spectral intensity exhibited by the amplicon. This enhanced fit is graphically depicted in contour plots shown in panels C and D of FIG. 23. Compared to the surface fitted by the Gaussian model, the estimates of the residual sum-of-squares, R_{SS}, and the standard deviation of the fit, SD, is significantly less in the TPS-fitted surface.

After calculating the total spectral intensity, the Gaussian-fitted surface yielded an estimate of approximately 6 percent less RFI than the TPS-fitted surface. While 6 percent difference in RFI may not seem too significant, it is nevertheless additional variability that contributes to spectral noise and may prevent the correct assignment of genotype. Moreover, the magnitude of error is expected to increase depending on (1) the resulting distortion of the amplicon and (2) the magnitude of RFI in the amplicon. Therefore, it is desirable to minimize surface-fitting errors whenever possible.

### IX. Normalization

The preceding transformations applied to the original photometric data have attenuated most of the random variation in signal intensity attributed primarily to electrophoresis- and detector-derived noise. However, variation in (1) sample-loading volumes and (2) concentration differences among amplicons in individual marker assays (i.e. the actual number of DNA molecules comprising a particular amplicon) and other variability also require correction 58 (FIG. 1). Once this procedure is done, preferably by the use of an internal normalization standard, the observed values of spectral intensity for each amplicon may be compared directly to each other to ascertain whether the amplicon was originally derived from one or two DNA templates (alleles). Recall that in the case of where no amplicon is present (null), no alleles were originally present and thus no amplicon is detected. As this comparison is relative across amplicons at an individual locus, a standard by which to gauge differences in spectral intensity among amplicons is mandatory.

Where pedigree information is known and provides certainty in knowledge of one or more monomorphic loci, a known monomorphic locus can be used to normalize the spectral intensities of all other amplicons using the normalization procedure set forth below. Where a monomorphic locus is not known a priori, e.g. when no pedigree or other suitable generic information is available, identification of a monomorphic locus must be performed before the normalization process can proceed.

In cases where monomorphic loci cannot be identified a priori, a minimum-variance search strategy preferably is implemented among putatively monomorphic loci, pMMₖ, k=1, 2, . . . , m (i.e. those loci j in Mᵢⱼ where for all i, Mᵢⱼ=1), to identify the locus with the least amount of variability in spectral intensity. The putatively monomorphic locus having the least amount of variability preferably is designated as a “true” monomorphic locus and used to normalize the spectral intensities of all other amplicons. The rationale behind this strategy is that if a locus is actually monomorphic, the variance in spectral intensity among amplicons at that locus should be smaller than if there was at least one amplicon with a polymorphic genotype present at that locus. Therefore, any locus j in Mᵢⱼ having at least one null homozygote (i.e. those loci j in Mᵢⱼ where for all i, at least one Mᵢⱼ=0) cannot be monomorphic and therefore are designated polymorphic, PMₗ, l=1, 2, . . . , p.

Having now omitted polymorphic sites from consideration of a candidate monomorphic locus, the selection of an actual monomorphic locus rests on the ability to distinguish among those amplicons having either homozygote present (preferably) or heterozygote genotypes. A difficulty arises in that it is the primary goal of this invention to distinguish among these genotypes, but to do so first requires the identification of a monomorphic locus. The circularity of this problem can be resolved by applying the rationale of the minimum-variance search strategy described above to each of the putatively monomorphic loci. Thus, it is expected that if a locus is actually monomorphic (i.e. comprised only of amplicons with [preferably] homozygote present genotypes), the variance in spectral intensity at this locus will be smaller than if there was at least one amplicon with a heterozygote genotype present at that locus.

In a preferred procedure for selecting a monomorphic locus, a coefficient of variation, ψ, in spectral intensity is first computed for all amplicons at each putative monomorphic locus. The coefficient of variation for spectral intensity at the k^{th} putatively monomorphic locus pMMₖ, k=1, 2, . . . , m, is given by

$${ϕ}_{k} = \frac{\sqrt{{S}_{{pMM}_{k}}^{2}}}{\overline{{pMM}_{k}}} \times 100. \qquad ( Equation LXXI )$$

In Equation LXXI, S_{pMM}_{ₖ}² is the k^{th} observed sample variance of spectral intensity in amplicons across all Sᵢ, i=1, 2, . . ., N samples and \overline{pMM}ₖ is the k^{th} average observed spectral intensity in amplicons across all Sᵢ, i=1, 2, . . . , N samples. Computed values of ψ for all k putatively monomorphic loci preferably are arranged in ascending order. Where this procedure is implemented in software, ordering all of the values of ψₖ may not be required.

The locus k exhibiting the smallest value of ψₖ is designated to be the monomorphic reference locus, rMM, provided that ψₖ is below an empirically derived magnitude, typically a value of ψ=12.000. Cross-correlation analyses of spectral intensities of amplicons at rMM to amplicons at loci with successively larger values of ψ may be performed to gauge the reliability of the original reference locus as a useful monomorphic locus. If a monomorphic reference locus cannot be defined, either by a priori knowledge or by using this procedure, it may not be possible to perform genotyping to discriminate among homozygote present and heterozygote genotypes. Where this procedure is implemented in software and a reference monomorphic locus cannot be defined, a message preferably is generated.

If a monomorphic reference locus is identified, preferably, the average spectral intensity, \overline{rMM}, of all amplicons at the reference locus is computed. This value is used to compute a normalizing coefficient, ηᵢ, for the amplicon in the i^{th} sample at the reference locus rMM

$${η}_{i} = \frac{{rMM}_{i}}{rMM} \qquad ( Equation LXXII )$$

where i=1, 2, . . . N. This procedure generates a set of normalizing coefficients which, when used to adjust the spectral intensity of corresponding amplicons at other loci, will remove the majority of variability in spectral intensity due to differences in (1) the individual marker assay, σ_{Marker assay}², and (2) the sample loading volume, σ_{Loading volume}². In a preferred implementation, each coefficient ηᵢ is divided into corresponding value of spectral intensity observed at other amplicon positions i for any other locus j. For example, the normalizing coefficients may be applied to any locus in pMMₖ, k=1, 2, . . . , m or PMₗ, l=1, 2, . . . p. The resulting [normalized] spectral intensity will more closely reflect values of expected spectral intensity if no variability in the marker assays or loading volumes existed. Thus, the normalizing coefficients, ηᵢ, computed from the i^{th} amplicon in rMM serve directly as the source of normalizing coefficients for all other loci in the entire sample. These normalizing coefficients form the basis by which genotypes are assigned to individual amplicons at other loci. This procedure is described in further detail below.

### X. Assignment of Genotypes to Individual Amplicons

Once the spectral intensities for amplicons at other loci are normalized, as described above, the resulting numerical values are prepared for genotyping 60 (FIG. 1). On a per locus basis, the spectral intensities of amplicons are expected to vary, possibly considerably, due to differences in the relative efficiency of the PCR reaction. It is therefore desirable to perform an additional normalization step to transform the normalized intra-locus spectral intensities to a common inter-locus scale.

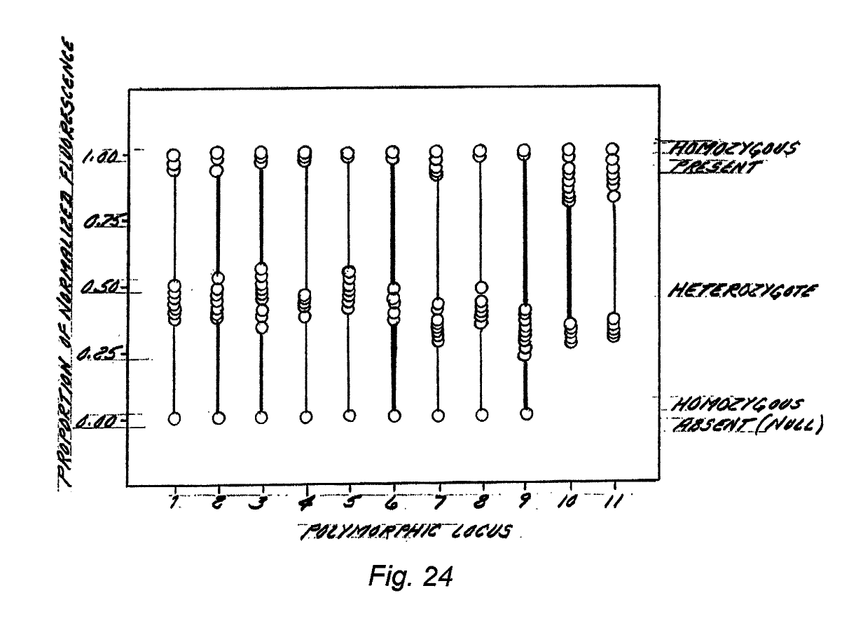

In a preferred implementation, the normalized values of spectral intensity are scaled to unity. For example, at a given locus, the amplicon exhibiting the maximum normalized spectral intensity is identified and divided into the spectral intensities of the remaining amplicons at that locus. Thus, the spectral intensity for each amplicon at that locus will be uniformly scaled to an RFI of 1.0. An advantage of scaling in this manner is that it allows simultaneous graphical visualization of any number of loci, e.g. FIG. 24, all of which are normalized to a common scale. Moreover, this procedure directly allows amplicon genotype assignments to be made based only on the relative proportion of normalized spectral intensity. FIG. 24 graphically illustrates the proportion of normalized spectral intensity computed for eleven loci using a preferred implementation of the normalization procedure described above. At all eleven loci, the three genotypic classes, homozygote present, heterozygote, and homozygote absent (null), are clearly separated from each other. Thus, visual inspection alone, without the reliance on additional statistical models, is sufficient enough to accurately assign genotypes (but see below).

In practice, it appears uncommon not to be able to unambiguously assign amplicons to a specific genotypic class using a simple graphical display such as illustrated in FIG. 24. In such cases where a genotype could not be unambiguously assigned using this procedure, it has been attributed to either (1) amplicon entry into the plateau-phase or (2) a poor fit of the preferred Gaussian model (due to amplicon distortion). In the latter case, refitting the photometric data of the amplicon in question with the TPS model and recalculating the spectral intensity resulted in an unambiguous assignment of genotype to that amplicon. In the former case, reducing the number of PCR cycles in the selective-phase of the fAFLP process resulted in an unambiguous assignment of genotype to those amplicons.

An additional advantage of this method is that it allows a user to gauge whether or not amplicons at a particular locus are entering the plateau phase. For example, referring to FIG. 24, the vast majority of heterozygous amplicons have not yet passed the inflection point (the proportion of normalized spectral intensity of 0.50 or greater) of the log-linear amplification phase illustrated in FIG. 3. Thus, presenting the data in this manner is useful in determining the optimum number of PCR cycles for a particular primer pair.

The invention also appears robust when inferring genotypes that are not derived exclusively from 0, 1 or 2 copies of an allele. In fact, the invention appears capable of distinguishing among any number of alleles. Such an instance has been observed in certain individuals for sex-linked loci in Domestic Chicken (Gallus gallus). For example, where the pedigree of parents and offspring is known, as was the case in a validation study, the origin and distribution of sex-chromosomes in offspring is predictable. At locus 559.562, we obtained genotypes in certain individuals consistent with 2, 3, and 4 allelic doses.

Nevertheless, an indeterminable genotype may arise using the preferred procedures of this invention. If it is determined that other identifiable processes are not responsible, genotype assignments may also be made statistically as briefly described below.

### Optional Gauging of Genotype Classification Probabilities

If desired, a statistically-based model can be used to optionally gauge or predict classification probabilities of genotypes assigned to specific classes. Many such models already exist. For example, both (1) genetic models based on expected allele distributions or (2) purely mathematical models, e.g., discriminant models, are suitable for gauging or predicting classification probabilities into a particular genotypic class. Thus, either aforementioned model can be used to statistically assign a genotype to an amplicon that could not be ascertained by visual inspection after normalized spectral intensities were scaled to unity and plotted in the manner depicted in FIG. 24. Either of these models can also assign a probability of correct assignment into a specific genotypic class for any spectral intensity representing a specific amplicon. Note that reference to the following two classification models is not inclusive of all available models to accomplish this task.

For example, the model discussed by Piepho and Koch (2000), Genetics, 155(3):1459-68, is a genetic model based on Bayesian expectations in normal mixtures of genotypes. Alternatively, a purely probabilistic discriminant function may also be used, e.g. the three-class discriminant function described by Fisher, 1938. Although both types of models are implemented in very different ways, each would be used to predictively classify the normalized spectral intensity of amplicons at loci into a defined number of classes, for example, homozygous present, heterozygous, and homozygous absent (null).

Nevertheless, the performance of either model to classify genotypes using spectral intensity data is almost completely unknown and has not been applied to data processed in the manner described herein. For example, genotype assignments presented in Piepho and Koch (2000) show a strong overlap of the probability distribution between the classes of bomozygous present and heterozygote, a phenomenon we generally do not observe. It is asserted that this is likely due to the failure to accommodate into their model important issues related to (1) spectral noise minimization, (2) spectral intensity estimation, and (3) spectral intensity normalization. Thus, it is believed that their method would be more accurate using data processed in accordance with methods of this invention.

### EXAMPLES

The following Examples are provided for illustrative purposes only. The Examples are included herein solely to aid in a more complete understanding of the presently described invention. The Examples do not limit the scope of the invention described or claimed herein in any fashion.

Sample validation. To validate the FAFLP codominant scoring procedure, a system where precise knowledge of parental and offspring genotypes was used. Two highly inbred lines of domestic chicken (Gallus gallus), a single Rhode-Island Red male and 8 Ancona females were crossed. A total of 48 F1 individuals were obtained from six hens. Nine autosomal and two sex-linked loci were used to validate the codominant fAFLP procedure. DNA samples of parents P:M3966 and P:F343 and 10 F1 individuals (Fl:M/F7163-7173) were randomized blindly with replacement and processed using the fAFLP genotyping procedure, such as that preferably described above. The results of the genotyping procedure are graphically illustrated in FIG. 24.

fAFLP Analysis. For fAFLP analysis of avian genomic DNA (e.g. G. gallus and Manacus manacus) two to three selective bases were sufficient to produce on average, 120 resolvable fragments 60 to 600 bases in length. For example, in M. manacus, each of twenty primer pairs, applied to individuals sampled at a small lek (7 males, 5 females), always demonstrated significant levels of polymorphism. The percentage of polymorphic loci for each of twenty primer pairs ranged from 15 to 47 percent, with an average of 23 percent.

It is understood that the various preferred embodiments shown and described above illustrate different possible features of the invention and the varying ways in which these features may be combined. Apart from combining the different features of the above embodiments in varying ways, other modifications are also considered to be within the scope of the invention. For example, the method can be used to analyze AFLP-generated fragments labeled with fluorescent dyes other than those specifically mentioned and labeled with dyes other than fluorescent dyes, e.g., radiolabels. The method can also be used to analyze AFLP fragments generated from nucleic acids other than DNA. For example, the fragments can be generated from mRNA. As noted above, the amplification products can be analyzed with a variety of instruments, including, but not limited to, automated DNA sequencers (both slab-gel and capillary-based), and densitometers.

What is claimed is:

1. A method of discriminating among genotypes comprising:
(a) providing a fragment of DNA, a detector, and photometric data obtained using the detector from the DNA fragment;
(b) compensating for error in the data; and
(c) using the error-compensated data to determine whether the genotype of the fragment is a homozygote or heterozygote.

2. The method according to claim 1 wherein step (b) comprises performing a baseline adjustment to the photometric data.

3. The method according to claim 2 wherein baseline adjustment comprises:
(1) arranging the photometric data into a plurality of groups where each have a plurality of data points that have values;
(2) determining the data point in each group that has the lowest value in the group;
(3) determining the slope between the lowest value data point of each pair of adjacent groups;
(4) determining an offset correction value for each data point between the lowest data point of each pair of adjacent groups using the slope;
(5) applying the offset value correction by subtracting it from the value of its associated data point.

4. The method according the claim 3 wherein the offset correction value for each data point is determined using the slope and the lowest data point of each pair of adjacent groups is determined using multipoint linear regression.

5. The method according to claim 2 wherein step (b) further comprises removing spectral overlap in the photometric data after baseline adjustment has been performed and thereafter removing spectral noise in the photometric data.

6. The method according to claim 1 wherein the detector comprises an electrophoresis detector that detects light emitted from the DNA fragment and generates the photometric data, wherein the error in the photometric data comprises at least one of detection-related error, electrophoresis-related error, marker assay-related error, and loading volume-related error, and, during step (b), a mathematical transformation and an image processing procedure is applied to the photometric data to compensate for a plurality of the errors, and during step (c) the error is removed and the remaining data is used in ascertaining the genotype of the fragment.

7. The method according to claim 1 wherein compensating for error in the photometric data in step (b) comprises attenuating noise in the photometric data and attenuating artifacts in the photometric data.

8. The method according to claim 7 wherein attenuating noise in the photometric data comprises transforming the photometric data into the frequency domain using a Fourier transform, truncating a portion of the transformed photometric data, and thereafter transforming the data into the time domain.

9. The method according to claim 8 wherein the Fourier Transform comprises a Discrete Hartley Transform and the data are transformed into the time domain using an inverse Discrete Hartley Transform.

10. The method according to claim 7 wherein attenuating artifacts in the data comprises applying an apodization function to the photometric data remaining after attenuating noise in the photometric data has been performed.

11. The method according to claim 10 wherein the apodization function comprises a Gaussian apodization function.

12. The method according to claim 1 wherein compensating for error in the photometric data in step (b) comprises:
(1) transforming the photometric data into the frequency domain using a Fourier Transform;
(2) attenuating noise in the photometric data; and
(3) transforming the photometric data into the time domain using an inverse Fourier Transform.

13. The method according to claim 12 wherein transforming the photometric data into the frequency domain using a Fourier Transform in step (1) produces a plurality of low-frequency components and a plurality of high-frequency components, and attenuating noise in the photometric data in step (2) comprises truncating at least one of the high-frequency components.

14. The method according to claim 13 wherein truncation of the at least one of the high-frequency components comprises removing at least one of the high-frequency components such that it is not transformed into the time domain in step (3).

15. The method according to claim 13 wherein truncation of at least one of the high-frequency components comprises truncating all of the high-frequency components that have a frequency within 20% of the high-frequency component having the highest frequency to remove high amplitude noise when the data are thereafter transformed back into the time domain in step (3).

16. The method according to claim 12 wherein transforming the photometric data into the frequency domain using a Fourier Transform in step (1) produces a plurality of low-frequency components and a plurality of high-frequency components, and attenuating noise in the photometric data in step (2) truncates at least one of the low-frequency components.

17. The method according to claim 16 wherein truncation of the least one of the low-frequency components comprises reducing the amplitude of at least one of the low-frequency components before it is transformed back into the time domain in step (3).

18. The method according to claim 17 wherein truncation of at least one of the low-frequency components comprises reducing the amplitude of the low-frequency component disposed at the lowest frequency by at least 40% to remove low-amplitude noise when the photometric data are thereafter transformed back into the time domain in step (3).

19. The method according to claim 12 wherein transforming the photometric data into the frequency domain using a Fourier Transform in step (1) produces a plurality of low-frequency components and a plurality of high-frequency components, and attenuating noise in the photometric data in step (2) truncates at least one of the high-frequency components by removing the at least one of the high-frequency components and truncates at least one of the low-frequency components by reducing the amplitude of the at least one of the low-frequency components.

20. The method according to claim 1 wherein compensating for error in the photometric data in step (b) comprises:
(1) transforming the photometric data into the frequency domain using a Discrete Hartley Transform;
(2) attenuating noise in the data by truncating a portion of the transformed photometric data;
(3) attenuating artifacts in the transformed photometric data by multiplying a Gaussian apodization function to the transformed photometric data; and
(4) retransforming the transformed photometric data into the time domain using an inverse Discrete Hartley Transform.

21. The method according to claim 1 wherein a first plurality of the DNA fragments are provided that each comprise an amplicon and that are each disposed in a first sample lane or capillary, a second plurality of the fragments are provided that each comprise an amplicon and that are each disposed in a second sample lane or capillary, the detector comprises a detection system that has a plurality of channels, and the photometric data comprises spectral intensity data obtained by the detection system from a plurality of labels attached to the first and second plurality of the fragments, and after step (a) performing the steps further comprising:
(1) reducing spectral noise in the photometric data;
(2) extracting lane-tracking information; and
(3) identifying a plurality of peaks in the photometric data.

22. The method according to claim 21 wherein the step of identifying a plurality of peaks in the photometric data comprises determining a location of an apex of one of the plurality of peaks by determining a region in the photometric data where spectral intensity steadily increases and identifying where this region ends by identifying where the spectral intensity decreases.

23. The method according to claim 22 wherein the step of identifying the apex of one of the plurality of peaks comprises identifying the scan number where the spectral intensity decreases and selecting the preceding scan number as the apex.

24. The method according to claim 1 wherein compensating for error in the photometric data in step (b) includes determining an edge of a peak using the photometric data, determining the location of an apex of the peak using the photometric data, and determining a width of the peak using the photometric data.

25. The method according to claim 24 wherein determining an edge of a peak comprises scanning the photometric data to determine the presence of a plurality of pairs of consecutively increasing data points of the photometric data that each holds a spectral intensity value.

26. The method according to claim 25 wherein the leading edge of a peak is determined when the spectral intensity of five consecutive data points increases.

27. The method according to claim 26 wherein after an edge of a peak is determined, an apex of the peak is determined by identifying the first data point having a decreasing value and selecting the location of the apex as being the preceding data point.

28. The method according to claim 27 wherein the value of the data point selected as being the apex has a value greater than an apex threshold or the location of the apex is discarded.

29. The method according to claim 24 wherein determining the width of the peak comprises estimating the peak full width at half the maximum value of the amplitude of the apex.

30. The method according to claim 1 wherein step (b) comprises locating a peak in the photometric data and replacing the peak with an idealized peak.

31. The method according to claim 30 wherein, before determining the idealized peak, defining the peak by locating an apex of the peak, determining a width of the peak, and determining an amplitude of the peak at the location of its apex, and the peak is replaced with the idealized peak using the location of the apex of the peak, the width of the peak and the amplitude of the peak.

32. The method according to claim 31 wherein the step of determining the width of the peak comprises determining a peak full width at half the maximum value of the amplitude of the peak at the location of its apex.

33. The method according to claim 31 wherein the idealized peak comprises a Gaussian function.

34. The method according to claim 1 wherein step (b) comprises locating a peak in the photometric data, fitting a Gaussian function to it, and replacing the peak with the Gaussian function.

35. The method according to claim 1 wherein step (b) comprises locating a peak in the photometric data, using nonlinear orthogonal distance regression to fit a Gaussian function thereto, and thereafter replacing the peak with the Gaussian function.

36. The method according to claim 1 further comprising obtaining photometric data from a set of standards each having a location and a known molecular weight, determining a sizing function for one of the standards by applying a locally-quadratic fit to one of the standards as well as to a number of other standards less than the total number of standards in the set, and thereafter using estimates obtained from the sizing function in estimating the molecular weight of the DNA fragment.

37. The method according to claim 36 wherein the locally-quadratic fit is performed using the data of one of the standards and weighted data of a plurality of adjacent standards.

38. The method according to claim 37 wherein DNA standards are used and the locally-quadratic fit for a particular standard is performed using the data of fewer than the entire complement of the standards.

39. The method according to claim 36 wherein the sizing function used in estimating the molecular weight of the DNA fragment is determined for one of the standards having a location in the vicinity of the DNA fragment and use of the the sizing function enables the molecular weight of the DNA fragement to be estimated to an accuracy of at least ±0.5 base pair.

40. The method according to claim 1 wherein step (b) comprises obtaining data from a set of standards each having a location and a known molecular weight, determining a fit for one of the standards by performing a weighted least-squares minimization thereof that comprises a weighting function that includes the contribution of a number of standards including the one of the standards and a plurality of standards adjacent to the one of the standards, and produces a plurality of estimates, with the number of standards contributed being selected so as to minimize the residual standard error of the weighted least-squares minimization, and thereafter using the plurality of estimates obtained from the least-squares minimization in estimating the molecular weight of the DNA fragment.

41. The method according to claim 40 wherein the number of standards contributed is dependent on a fraction of the standards used in the weighted least-squares minimization selected so as to minimize the residual standard error.

42. The method according to claim 1 wherein step (b) comprises obtaining data from a set of standards each having a location and a known molecular weight, performing a least squares minimization on the data to produce a plurality of estimates, and thereafter estimating a molecular weight of the DNA fragment using a quadratic equation, the plurality of estimates, and a location of a peak of the DNA fragment.

43. The method according to claim 42 wherein the plurality of estimates and the location of the peak of the DNA fragment are inputted into the quadratic equation.

44. The method according to claim 1 wherein there are a plurality of samples that each have a plurality of the DNA fragments at different molecular weights with one of the samples comprising a sample that includes a plurality of DNA fragments used as standards, obtaining an intensity value for each DNA fragment, identifying monomorphic locus, and normalizing the intensity values of the DNA fragments at all other loci using the intensity values of the DNA fragments at the monomorphic locus.

45. The method according to claim 44 wherein identifying a monomorphic locus further comprises designating each locus that has a DNA fragment in each sample as a putatively monomorphic locus and designating the putatively monomorphic locus having the lowest variability as being a monomorphic locus of a specific molecular weight.

46. The method according to claim 44 wherein identification of the monomorphic locus of a specific molecular weight further comprises locating the locus with a DNA fragment in each sample that has the lowest variability compared to all other loci of differing molecular weights that have a DNA fragment in each sample.

47. The method according to claim 44 wherein normalizing further comprises determining a normalizing coefficient for each sample using the intensity value of each DNA fragment of the monomorphic locus and applying the normalizing coefficient for the sample to each intensity value of each DNA fragment of the sample.

48. The method according to claim 47 wherein the normalizing coefficient determined for each sample is obtained by dividing the intensity value of a fragment at the monomorphic locus of the sample with an average of the intensity values of the DNA fragment at each sample at the monomorphic locus, the normalizing coefficient determined for each sample is applied by dividing each intensity value of each DNA fragment of that sample with the normalizing coefficient determined for that sample producing a normalized result, and each normalized result is substituted for the intensity value associated with the particular DNA fragment of that sample.

49. The method according to claim 1 wherein there are a plurality of samples that each have a plurality of the DNA fragments at different loci with one of the samples comprising a sample that includes a plurality of DNA fragments used as standards, obtaining an intensity value for each DNA fragment, normalizing the intensity values of the DNA fragments at all loci, scaling the normalized intensity values, and assigning a genotype to each DNA fragment based on the scaled normalized intensity.

50. The method according to claim 49 wherein the normalized values are scaled to unity and (1) a homozygous absent genotype is assigned to each DNA fragment having a scaled and normalized intensity value of about zero, (2) a homozygous present genotype is assigned to each DNA fragment having a scaled and normalized intensity value of about one, and (3) a heterozygote genotype is assigned to each DNA fragment having an intermediate scaled and normalized intensity value.

51. The method according to claim 1 wherein there are a plurality of samples that each have a plurality of the DNA fragments from different loci with one of the samples comprising a sample that includes a plurality of DNA fragments used as standards, obtaining an intensity value for each DNA fragment, normalizing the intensity values of the DNA fragments, and, for each locus, assigning a homozygous present genotype to each DNA fragment having a normalized intensity value that is at or about a maximum normalized intensity value, and assigning a heterozygote genotype to each DNA fragment having a normalized intensity value that is about half the maximum normalized intensity value.

52. The method according to claim 51 further comprising assigning a homozygous absent genotype to each DNA fragment having a normalized intensity value of about zero.

53. The method according to claim 1 wherein a plurality of the fragments are provided that each comprise an amplicon, the detector comprises a fluorescent detection system, and the photometric data comprises luminuous intensity data obtained from excited fluorophores carried by the fragments, and in step (b) performing the steps further comprising:
(1) creating a spectral baseline;
(2) removing spectral overlap;
(3) reducing noise spikes;
(4) assigning a molecular weight to each one of the amplicons;
(5) estimating the spectral intensity for each amplicon; and
(6) normalizing the spectral intensities of all of the amplicons; and
during step (c) assigning a genotype to each amplicon using the normalized spectral intensities.

54. The method according to claim 1 wherein a first plurality of the DNA fragments are provided that each comprise an amplicon and that are each disposed in a first sample lane or capillary, a second plurality of the DNA fragments are provided that each comprise an amplicon and that are each disposed in a second sample lane or capillary, the detector comprises a fluorescent detection system that has a plurality of channels, and the photometric data comprises spectral intensity-related data obtained by the fluorescent detection system from fluorophores attached to the first and second plurality of the DNA fragments, and during step (b) performing the steps further comprising:
(1) creating a spectral baseline;
(2) removing spectral overlap between detector channels;
(3) attenuating spectral noise;
(4) creating false-color images;
(5) assigning a molecular weight to each one of the DNA fragments;
(6) determining sample lanes;
(7) estimating a spectral intensity for each one of the DNA fragments;
(8) normalizing the spectral intensities of all of the amplicons; and
during step (c) assigning a genotype using the normalized spectral intensities.

55. A method of processing photometric data comprising:
(a) providing a plurality of fragments of DNA, a detector, and photometric data obtained using the detector from the DNA fragments;
(b) adjusting a baseline of the photometric data;
(c) reducing spectral noise;
(d) identifying a peak in the photometric associated with each one of a plurality of the DNA fragments;
(e) sizing the DNA fragments;
(f) estimating spectral intensity for each fragment;
(g) normalizing the spectral intensities;
(h) genotyping each fragment using the normalized spectral intensities.

56. The method of processing photometric data according to claim 55 wherein adjusting a baseline of the photometric data is done using multipoint linear regression.

57. The method of processing photometric data according to claim 55 wherein reducing spectral noise comprises transforming the photometric data into the frequency domain, truncating a portion of the transformed photometric data, and thereafter transforming the portion of the transformed photometric data that remains after truncation back into the time domain.

58. A method of discriminating a genotype comprising:
(a) compensating for error in data obtained from a DNA fragment; and
(b) using the error-compensated data to determine whether the genotype of the fragment is a homozygote or heterozygote.

59. The method according to claim 58 wherein compensating for error in step (a) comprises:
(1) arranging the photometric data into a plurality of groups that each have a plurality of data points that each have a value;
(2) determining the data point in each group that has the lowest value in its group;
(3) determining the slope between the lowest value data point of each pair of adjacent groups;
(4) determining an offset correction value for each data point between the lowest data point of each pair of adjacent groups using the slope; and
(5) applying the offset value correction by subtracting it from the value of its associated data point.

60. The method according to claim 58 wherein compensating for error in step (a) comprises:
(1) transforming the data from a time domain into a frequency domain using a Fourier transform;
(2) truncating a portion of the transformed data;
(3) transforming the data from the frequency domain back into the time domain using an inverse Fourier transform.

61. The method according to claim 58 wherein data are obtained from a plurality of DNA fragments and compensating for error in step (a) comprises:
(1) transforming the data from a time domain into a frequency domain using a Fourier transform;
(2) truncating a portion of the transformed data beyond a cutoff frequency;
(3) truncating a portion of the transformed data beyond a cutoff amplitude;
(4) applying an apodization function to the transformed data remaining after truncation; and
(5) transforming the data remaining after truncation and apodization from the frequency domain into the time domain using an inverse Fourier transform.

62. The method according to claim 61 wherein transformation of the data in step (1) produces a plurality of frequency components, wherein truncation of the transformed data in step (2) comprises removing any frequency component having a frequency above the cutoff frequency, wherein truncation of the transformed data in step (3) comprises removing that portion of any frequency component having an amplitude greater than the cutoff amplitude, and the apodization function is applied to the frequency components that remain after truncation has been performed in steps (2) and (3).

63. The method according to claim 58 wherein data are obtained from a plurality of DNA fragments and compensating for error in step (a) comprises locating a plurality of peaks in the data and fitting an idealized peak to each one of the plurality of peaks.

64. The method according to claim 58 wherein data are obtained from a plurality of DNA fragments and compensating for error in step (a) further comprises locating a plurality of peaks by locating an apex of each one of the plurality of peaks, locating a width of the each one of the plurality of peaks, and locating an amplitude at the apex of each one of the plurality of peaks; using the apex location, width, and apex amplitude of each one of the plurality of peaks to fit a Gaussian peak thereto; and thereafter replacing each one of the plurality of peaks with the Gaussian peak fitted thereto.

65. The method according to claim 58 wherein data are obtained from a plurality of DNA fragments and compensating for error in step (a) further comprises locating a plurality of peaks by locating an apex, a variance, and an apex amplitude of each one of the plurality of peaks; performing non-linear orthogonal distance regression using the apex location, width, and apex amplitude of each one of the plurality of peaks to fit a Gaussian peak thereto; and thereafter replacing each one of the plurality of peaks with one of the Gaussian peaks fitted thereto.

66. The method according to claim 58 wherein a first set of data are obtained from a plurality of DNA fragments, a second set of data are obtained from a plurality of DNA fragment standards, and compensating for error in step (a) comprises, for each one of the standards, determining a sizing function by applying a locally quadratic fit to one of the standards using the data of the one of the standards and the data of a plurality of standards other than the one of the standards; and thereafter using the sizing function of one of the standards in estimating the molecular weight of the plurality of DNA fragments.

67. The method according to claim 58 wherein a first set of data are obtained from a plurality of DNA fragments, a second set of data are obtained from a plurality of DNA fragment standards, and compensating for error in step (a) comprises, for each one of the standards, performing a weighted least squares minimization that includes a weighting function that weights the contribution of a number of the standards so as to include the complete contribution of the one of the standards and a lesser contribution of a plurality of the standards located adjacent to the one of the standards; wherein the number of standards contributed to each weighting function is selected so as to minimize the residual standard error of the weighted least squares minimization; and using the weighting function to produce a plurality of estimates that are used in determining a molecular weight of one of the plurality of DNA fragments.

68. A method of discriminating a genotype comprising:
(a) providing a plurality of samples that each have a plurality of DNA fragments from a plurality of loci, a plurality of DNA fragment standards, a detector, and photometric data obtained from the plurality of DNA fragments and the plurality of DNA fragment standards using the detector;
(b) performing a baseline adjustment to the photometric data;
(c) removing spectral overlap in the photometric data;
(d) transforming the photometric data from a time domain into a frequency domain using a Fourier transform, truncating a portion of the transformed photometric data, and thereafter transforming the transformed photometric data remaining after truncation from the frequency domain back into the time domain using an inverse Fourier transform;
(e) identifying a plurality of peaks in the photometric data and fitting a Gaussian peak to each one of the plurality of peaks;
(f) performing a plurality of locally weighted quadratic regressions on the plurality of peaks to produce a plurality of molecular weight sizing functions;
(g) estimating the molecular weight of each one of the plurality of DNA fragments using one of the plurality of molecular weight sizing functions;
(h) binning the molecular weights of the plurality of DNA fragments;
(i) designating each locus having a fragment present in each one of the samples as putatively monomorphic;
(j) estimating a spectral intensity for each one of the plurality of DNA fragments;
(k) designating the putative monomorphic locus having the least amount of variability in spectral intensity as a reference monomorphic locus;
(l) determining a normalizing coefficient using the spectral intensities of the DNA fragment of the reference monomorphic locus;
(m) using the normalizing coefficient to normalize the spectral intensities of all of the plurality of DNA fragments; and
(n) using the normalized spectral intensities to genotype the plurality of DNA fragments.

69. A method of discriminating a genotype comprising:
(a) providing a plurality of samples that each have a plurality of DNA fragments at a plurality of loci, a plurality of DNA fragment standards, a detector, and photometric data obtained from the plurality of DNA fragments and the plurality of DNA fragment standards using the detector;
(b) identifying a plurality of peaks in the photometric data;
(c) performing a plurality of locally weighted quadratic regressions on the plurality of peaks to produce a plurality of molecular weight sizing functions;
(d) estimating the molecular weight of each one of the plurality of DNA fragments using one of the plurality of molecular weight sizing functions;
(e) binning the molecular weights of the plurality of DNA fragments;
(f) designating each locus that has a DNA fragment present in each one of the samples as putatively monomorphic;
(g) estimating a spectral intensity for each one of the plurality of DNA fragments;
(h) designating the putative monomorphic locus having the least amount of variability in spectral intensity as a reference monomorphic locus;
(i) determining a normalizing coefficient using the spectral intensities of the DNA fragment of the reference monomorphic locus;
(j) using the normalizing coefficient to normalize the spectral intensities of all of the plurality of DNA fragments; and
(k) using the normalized spectral intensities to genotype the plurality of DNA fragments.

70. A method of discriminating a genotype comprising:
(a) providing a plurality of samples that each have a plurality of DNA fragments at a plurality of loci, a plurality of DNA fragment standards, a detector, and photometric data obtained from the plurality of DNA fragments and the plurality of DNA fragment standards using the detector;
(b) transforming the photometric data from a time domain into a frequency domain using a Fourier transform, truncating a portion of the transformed photometric data, and thereafter transforming the data remaining after truncation from the frequency domain back into the time domain using an inverse Fourier transform;
(c) identifying a plurality of peaks in the photometric data and fitting an idealized peak to each one of the plurality of peaks;
(d) determining a plurality of molecular weight sizing functions using the photometric data from the plurality of DNA fragment standards;
(e) estimating the molecular weight of each one of the plurality of DNA fragments using one of the plurality of molecular weight sizing functions;
(f) binning the molecular weights of the plurality of DNA fragments;
(g) designating the locus having the least amount of variability as a reference monomorphic locus;
(h) normalizing all of the DNA fragments using a normalization coefficient determined using the reference monomorphic locus;
(i) genotyping the plurality of DNA fragments.

71. A method of discriminating a genotype comprising:
(a) providing a plurality of samples that each have a plurality of DNA fragments at a plurality of loci, a plurality of DNA fragment standards, a detector, and photometric data obtained from the plurality of DNA fragments and the plurality of DNA fragment standards using the detector;
(b) estimating a molecular weight for each one of the plurality of DNA fragments;
(c) binning the molecular weights;
(d) designating the locus having the least amount of variability as a reference monomorphic locus;
(e) normalizing the DNA fragments using a normalization coefficient obtained from the reference monomorphic locus;
(f) genotyping the plurality of DNA fragments.

72. A method of discriminating among genotypes comprising:
(a) providing a plurality of samples that each have a plurality of DNA fragments at a plurality of loci, a plurality of DNA fragment standards, a detector, and data obtained from the plurality of DNA fragments and the plurality of DNA fragment standards using the detector;
(b) transforming the data from a time domain into a frequency domain using a Fourier transform, truncating a portion of the transformed data, and thereafter transforming the data remaining after truncation from the frequency domain back into the time domain using an inverse Fourier transform; and
(c) genotyping the plurality of DNA fragments.

73. A method of discriminating among genotypes comprising:
(a) providing a plurality of samples that each have a plurality of DNA fragments disposed in a gel matrix from a plurality of loci, a plurality of DNA fragment standards, a detector, and data obtained from the plurality of DNA fragments and the plurality of DNA fragment standards using the detector;
(b) estimating spectral intensity of each one of the plurality of DNA fragment using a generalized surface-fitting function to model the distribution of DNA fragments in a gel matrix; and
(c) assigning a genotype to each one of the plurality of DNA fragments.

74. The method according to claim 73 wherein the generalized surface fitting function comprises a thin-plate spline function.

75. The method according to claim 73 wherein the generalized surface fitting function comprises a Gaussian function.

76. A method of discriminating among genotypes comprising:
(a) providing at least one amplicon, a detector, and photometric data obtained using the detector from the at least one amplicon;
(b) compensating for error in the data; and
(c) using the error-compensated data to determine whether the genotype of the at least one amplicon is a homozygote or heterozygote;
(d) using the error-compensated data to determine the original copy number of DNA templates from which the at least one amplicon was derived.
